(19) United States
(12) Patent Application Publication
(10) Pub. No.: US 2023/0255156 A1
(43) Pub. Date: Aug. 17, 2023

(54) METHODS FOR IDENTIFYING AND SELECTING MAIZE PLANTS WITH RESISTANCE TO NORTHERN CORN LEAF BLIGHT

(71) Applicants: KWS SAAT SE & Co. KGaA, Einbeck (DE); UNIVERSITY OF ZURICH, Zurich (CH)

(72) Inventors: Daniela SCHEUERMANN, Einbeck (DE); Milena OUZUNOVA, Gottingen (DE); Bettina KESSEL, Einbeck (DE); Beat KELLER, Zurich (CH); Ping YANG, Zurich (CH)

(73) Assignees: KWS SAAT SE & Co. KGaA, Einbeck (DE); UNIVERSITY OF ZURICH, Zurich (CH)

(21) Appl. No.: 18/015,116

(22) PCT Filed: Jul. 14, 2021
(86) PCT No.: PCT/EP2021/069552

§ 371 Date: Jan. 9, 2023

(30) Foreign Application Priority Data
Jul. 14, 2020 (EP) 20185759.6

(51) Int. Cl. A01H 1/04; C12Q 1/6895; A01H 1/00; A01H 6/46; A01H 5/10

(52) U.S. Cl. CPC A01H 1/045; C12Q 1/6895; A01H 1/1255; A01H 6/4684; A01H 5/10; C12Q 2600/13; C12Q 2600/156

(57) ABSTRACT

The present invention relates to maize plants having increased pathogen resistance or tolerance, in particular increased resistance or tolerance to pathogens causing Northern Corn Leaf Blight, i.e. Exserohilum turcicum. Such maize plants are characterized by particular molecular markers The invention further relates to methods for identifying such maize plants.

### FIELD OF THE INVENTION

The invention relates to maize plants having increased pathogen resistance or tolerance, in particular increased resistance or tolerance to Exserohilum turcicum.

### BACKGROUND OF THE INVENTION

In maize (Zea mays L.), there are a large number of fungal pathogens which cause leaf diseases. The fungus which can cause by far the most damage under tropical and also under temperate climatic conditions, such as those in large parts of Europe and North America as well as in Africa and India, is known as Helminthosporium turcicum or synonymously as Exserohilum turcicum (Pass.) Leonard and Suggs (teleomorph: Setosphaeria turcica (Luttrell) Leonard & Suggs). H. turcicum is the cause of the leaf spot disease known as “Northern Corn Leaf Blight” (NCLB), which can occur in epidemic proportions during wet years, attacking vulnerable maize varieties and causing a great deal of damage and considerable losses of yield of 30% and more over wide areas (Perkins & Pedersen, 1987; Raymundo & Hooker, 1981a; Ullstrup & Miles, 1957). Since the 1970s, then, natural resistance in genetic material has been sought. Currently, quantitative and qualitative resistances are known. While the oligo- or polygenically inherited quantitative resistance appears incomplete and non-specific as regards race in the phenotype and is influenced by additional and partially dominant genes, qualitative resistance is typically race-specific and can be inherited through individual, mostly dominant genes such as Ht1, Ht2, Ht3, Htm1 or Htn1 (Lipps et al., 1997; Welz & Geiger, 2000). Backcrosses in many frequently used inbred maize lines such as W22, A619, B37 or B73 have successfully brought about introgression of the HT genes, where they exhibit a partial dominance and expression as a function of the respective genetic background (Welz, 1998).

Despite this complex genetic architecture of NCLB resistance in maize, until now principally the use of the Ht1 gene in maize together with a partial quantitative resistance has been sufficient to control helminthrosporiosis (Welz, 1998). The basis for this is that globally, races 0 and 1 of H. turcicum are most prevalent (approximately 55%) (Lipps et al., 1997; Ferguson & Carson, 2007), while other races such as 2N and 23N are only rare and even then in a geographically restricted areas (Moghaddam & Pataky, 1994; Jordan et al., 1983; Lipps & Hite, 1982; Thakur et al., 1989; Welz, 1998). Race 0 is avirulent having regard to a maize plant with Ht1, so that when provided with a suitable quantitative resistance, it exhibits a sufficient general resistance to NCLB. However, many studies have reported an increasing dissemination of the less common races (Jordan et al., 1983; Welz, 1998; Pratt & Gordon, 2006). The reasons for this are linked to the population dynamic of a pathogen which allows changes in pathogen virulence by new mutations on avirulence genes and new combinations of available virulence genes. This can lead to the occurrence of new, sometimes more aggressive pathogenic races. In Brazil, for example, the H. turcicum population already appears to be substantially more diverse having regard to the race composition than, for example, in North America. Gianasi et al. (1996) reported H. turcicum races which have already broken through the resistance conferred by the Ht1 gene. In addition, there is the instability of the resistance genes to certain environmental factors such as temperature and light intensity in some climate zones (Thakur et al., 1989). This development has the consequence that globally the use of novel HT resistance genes or such to which, until now, little attention has been paid for the production of commercial maize plants is growing in importance in order to target a broader and more long-lasting resistance to H. turcicum in maize. Initial approaches in this regard were attempted as early as 1998 by Pataky et al. The NCLB resistance in sh2 elite maize was improved by using a combination of Ht1 and Htn1.

Meanwhile many sources of new NCLB resistance in form of QTLs are known (Galiano-Carneiro, A. L., & Miedaner, T. (2017). Genetics of Resistance and Pathogenicity in the Maize/Setosphaeria turcica Pathosystem and Implications for Breeding. Frontiers in plant science, 8, 1490), however the functional validation of these QTLs as well as the molecular characterisation is lacking—causative genes are not known. Marker development is very difficult and the optimized use of these resistances in breeding is limited. Nevertheless there is a continuing need for the well-characterized resistance genes which can then be used for the development of novel NCLB-resistant plant varieties.

It is thus an objective of the present invention to identify and/or further characterize plant resistance genes encoding polypeptides conferring or increasing resistance against a fungal pathogen, such as Helminthosporium turcicum.

### SUMMARY OF THE INVENTION

The present invention relates to maize plants having increased pathogen resistance or tolerance, in particular increased resistance or tolerance to pathogens causing Northern Corn Leaf Blight, i.e. Exserohilum turcicum (also known as Helminthosporium turcicum). Such maize plants can be characterized as having particular molecular markers, as defined herein elsewhere. Such maize plants can be characterized as having a particular resistance gene, as defined herein elsewhere. The invention further relates to methods for generating such maize plants, as well as methods for identifying such maize plants. The invention further relates to the use of such maize plants in controlling pathogen infestation. The invention also relates to (isolated) polynucleic acids, such as polynucleic acids comprising or encoding the molecular markers, or resistance genes or polynucleic acids specifically hybridizing therewith, as well as the use of such polynucleic acids for identifying or generating the maize plants of the invention. The invention also relates to maize plants or plant parts thereof, obtained by or obtainable by the method as described herein, as well as maize plants or plant parts thereof comprising the polynucleic acids as described herein.

The markers of the present invention are in particular suitable for identifying both the HT2 allele and the HT3 allele, in contrast to previously known markers. Hence the diagnostic value of the markers of the present invention is much higher, as the markers can be used in a wide range of genotypes. The markers of the present invention (as well as their combinations) are in particular unique for both the HT2 and HT3 alleles.

The present invention is in particular captured by any one or any combination of one or more of the below numbered statements 1 to 157, with any other statement and/or embodiments.

1. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying a polynucleic acid (comprised in a chromosomal interval of maize chromosome 8 between molecular markers PZE-108092843 and PZA-003182005) comprising any one or more of molecular marker (allele) MA0045, MA0062, MA0063, MA0070, MA0071, and MA0064 in a maize plant or plant part.

2. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying a polynucleic acid (comprised in a chromosomal interval of maize chromosome 8 between molecular markers PZE-108092843 and PZA-003182005) comprising any two or more of molecular marker (allele) MA0045, MA0062, MA0063, MA0070, MA0071, and MA0064 in a maize plant or plant part.

3. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying a polynucleic acid (comprised in a chromosomal interval of maize chromosome 8 between molecular markers PZE-108092843 and PZA-003182005) comprising any three or more of molecular marker (allele) MA0045, MA0062, MA0063, MA0070, MA0071, and MA0064 in a maize plant or plant part.

4. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying a polynucleic acid (comprised in a chromosomal interval of maize chromosome 8 between molecular markers PZE-108092843 and PZA-003182005) comprising molecular marker (allele) MA0045, MA0062, MA0063, MA0070, MA0071, and MA0064 in a maize plant or plant part.

5. The method according to any of statements 1 to 4, wherein said polynucleic acid further comprises molecular marker (allele) PZE-108095339.

6. The method according to any of statements 1 to 4, wherein said polynucleic acid further comprises molecular marker (allele) Affx-91328160.

7. The method according to any of statements 1 to 6, comprising screening for the presence of, detecting, or identifying any one or more of molecular marker (allele) MA0045, MA0062, MA0063, MA0070, MA0071, and MA0064, and optionally PZE-108095339 and/or Affx-91328160 in a maize plant or plant part.

8. The method according to any of statements 1 to 6, comprising screening for the presence of, detecting, or identifying any two or more of molecular marker (allele) MA0045, MA0062, MA0063, MA0070, MA0071, and MA0064, and optionally PZE-108095339 and/or Affx-91328160 in a maize plant or plant part.

9. The method according to any of statements 1 to 6, comprising screening for the presence of, detecting, or identifying any three or more of molecular marker (allele) MA0045, MA0062, MA0063, MA0070, MA0071, and MA0064, and optionally PZE-108095339 and/or Affx-91328160 in a maize plant or plant part.

10. The method according to any of statements 1 to 6, comprising screening for the presence of, detecting, or identifying any four or more of molecular marker (allele) MA0045, MA0062, MA0063, MA0070, MA0071, MA0064, and optionally PZE-108095339 and/or Affx-91328160 in a maize plant or plant part.

11. The method according to any of statements 1 to 6, comprising screening for the presence of, detecting, or identifying any five or more of molecular marker (allele) MA0045, MA0062, MA0063, MA0070, MA0071, MA0064, and optionally PZE-108095339 and/or Affx-91328160 in a maize plant or plant part.

12. The method according to any of statements 1 to 6, comprising screening for the presence of, detecting, or identifying molecular marker (allele) MA0045, MA0062, MA0063, MA0070, MA0071, MA0064, PZE-108095339 and Affx-91328160 in a maize plant or plant part.

13. The method according to any of statements 1 to 6, comprising screening for the presence of, detecting, or identifying molecular marker (allele) MA0045, MA0062, MA0063, MA0070, MA0071, and MA0064.

14. The method according to any of statements 1 to 13, wherein said polynucleic acid is flanked by and includes molecular marker (allele) MA0045 and Affx-91328160.

15. The method according to any of statements 1 to 13, wherein said polynucleic acid is flanked by and includes molecular marker (allele) MA0045 and PZE-108095339.

16. The method according to any of statements 1 to 13, wherein said polynucleic acid is flanked by and includes molecular marker (allele) MA0045 and MA0064, MA0045 and MA0063, MA0045 and MA0070, or MA0045 and MA0071.

17. The method according to any of statements 1 to 16, wherein said polynucleic acid does not comprise (HT2 or HT3) molecular marker (allele) PZE-108092843 and/or PZA-003182005.

18. The method according to any of statements 1 to 16, wherein said polynucleotide does not comprise (HT2 or HT3) molecular marker (allele) PZE-108094590 and/or PZE-108096610.

19. The method according to any of statements 1 to 16, wherein said polynucleotide does not comprise (HT2 or HT3) molecular marker (allele) PZE-108094590 and/or PZE-108096469.

20. The method according to any of statements 1 to 16, wherein said polynucleotide does not comprise (HT2 or HT3) molecular marker (allele) MA0043 and/or MA0025.

21. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying any one or more of molecular marker (allele) MA0045, MA0062, MA0063, MA0070, MA0071, and MA0064, and optionally PZE-108095339 and/or Affx-91328160 (comprised in a chromosomal interval of maize chromosome 8 between molecular markers PZE-108092843 and PZA-003182005) in a maize plant or plant part.

22. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying any two or more of molecular marker (allele) MA0045, MA0062, MA0063, MA0070, MA0071, and MA0064, and optionally PZE-108095339 and/or Affx-91328160 (comprised in a chromosomal interval of maize chromosome 8 between molecular markers PZE-108092843 and PZA-003182005) in a maize plant or plant part.

23. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying any three or more of molecular marker (allele) MA0045, MA0062, MA0063, MA0070, MA0071, and MA0064, and optionally PZE-108095339 and/or Affx-91328160 (comprised in a chromosomal interval of maize chromosome 8 between molecular markers PZE-108092843 and PZA-003182005) in a maize plant or plant part.

24. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying any four or more of molecular marker (allele) MA0045, MA0062, MA0063, MA0070, MA0071, MA0064, and optionally PZE-108095339 and/or Affx-91328160 (comprised in a chromosomal interval of maize chromosome 8 between molecular markers PZE-108092843 and PZA-003182005) in a maize plant or plant part.

25. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying any five or more of molecular marker (allele) MA0045, MA0062, MA0063, MA0070, MA0071, MA0064, and optionally PZE-108095339 and/or Affx-91328160 (comprised in a chromosomal interval of maize chromosome 8 between molecular markers PZE-108092843 and PZA-003182005) in a maize plant or plant part.

26. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying molecular marker (allele) MA0045, MA0062, MA0063, MA0070, MA0071, MA0064, and optionally PZE-108095339 and/or Affx-91328160 (comprised in a chromosomal interval of maize chromosome 8 between molecular markers PZE-108092843 and PZA-003182005) in a maize plant or plant part.

27. A method for identifying a maize plant or plant part, preferably a maize plant or plant part having (increased) pathogen resistance and/or tolerance, preferably wherein said pathogen is Exserohilum turcicum, comprising screening for the presence of, detecting, or identifying molecular marker (allele) MA0045, MA0062, MA0063, MA0070, MA0071, and MA0064 (comprised in a chromosomal interval of maize chromosome 8 between molecular markers PZE-108092843 and PZA-003182005) in a maize plant or plant part.

28. The method according to any of statements 21 to 27, further comprising screening for the presence of, detecting, or identifying molecular marker (allele) PZE-108092843 and/or PZA-003182005.

29. The method according to any of statements 21 to 27, further comprising screening for the presence of, detecting, or identifying molecular marker (allele) PZE-108094590 and/or PZE-108096610.

30. The method according to any of statements 21 to 27, further comprising screening for the presence of, detecting, or identifying molecular marker (allele) PZE-108094590 and/or PZE-108096469.

31. The method according to any of statements 21 to 27, further comprising screening for the presence of, detecting, or identifying molecular marker (allele) MA0043 and/or MA0025.

32. The method according to any of statements 1 to 31, further comprising the step of identifying and optionally selecting said plant or plant part if said one or more, two or more, three or more, four or more or five or more of molecular marker (allele) MA0045, MA0062, MA0063, MA0070, MA0071, MA0064, and optionally PZE-108095339 and/or Affx-91328160 is present.

33. The method according to any of statements 1 to 31, further comprising the step of identifying and optionally selecting said plant or plant part if molecular marker (allele) PZE-108092843 and/or PZA-003182005, PZE-108092843 and/or PZA-003182005, PZE-108094590 and/or PZE-108096469, and/or MA0043 and/or MA0025 is absent.

34. The method according to any of statements 1 to 33, wherein:
MA0045 is a SNP which is A at a position corresponding to position 156,789,751 bp of chromosome 8 of the B73 reference genome AGPv4;
MA0062 is a SNP which is G at a position corresponding to position 156,772,431 bp of chromosome 8 of the B73 reference genome AGPv4;
MA0063 is a SNP which is T at a position corresponding to position 156,543,061 bp of chromosome 8 of the B73 reference genome AGPv4;
MA0070 is a SNP which is T at a position corresponding to position 156,694,565 bp of chromosome 8 of the B73 reference genome AGPv4;
MA0071 is a SNP which is C at a position corresponding to position 156,694,641 bp of chromosome 8 of the B73 reference genome AGPv4;
MA0064 is a SNP which is C at a position corresponding to position 156,541,890 bp of chromosome 8 of the B73 reference genome AGPv4;
PZE-108095339 is a SNP which is G at a position corresponding to position 156,378,424 bp of chromosome 8 of the B73 reference genome AGPv4;
Affx-91328160 is a SNP which is G at a position corresponding to position 156,372,823 bp of chromosome 8 of the B73 reference genome AGPv4;
PZE-108092843, which is a SNP which is C at a position corresponding to position 154,456,106 bp of chromosome 8 of the B73 reference genome AGPv4;
PZE-108094590, which is a SNP which is G at a position corresponding to position 155,860,812 bp of chromosome 8 of the B73 reference genome AGPv4;
MA0043, which is a SNP which is A at a position corresponding to position 155,999,733 bp of chromosome 8 of the B73 reference genome AGPv4;
MA0025, which is a SNP which is G at a position corresponding to position 156,967,599 bp of chromosome 8 of the B73 reference genome AGPv4;
PZE-108096469, which is a SNP which is G at a position corresponding to position 157,266,475 bp of chromosome 8 of the B73 reference genome AGPv4;
PZE-108096610, which is a SNP which is G at a position corresponding to position 157375673 of chromosome 8 of the B73 reference genome AGPv4; or
PZA-003182005, which is a SNP which is A at a position corresponding to position 158,167,578 bp of chromosome 8 of the B73 reference genome AGPv4.

35. The method according to any of statements 1 to 34, which is a method for identifying a maize plant or plant part having (increased) resistance to a pathogen.

36. The method according to any of statements 1 to 35, which is a method for identifying a maize plant or plant part having (increased) resistance to a fungal pathogen.

37. The method according to any of statements 35 to 36, wherein said pathogen is a Exserohilum sp.

38. The method according to any of statements 35 to 37, wherein said pathogen is Exserohilum turcicum.

39. The method according to any of statements 35 to 37, wherein said pathogen is Exserohilum turcicum causing NCLB.

40. The method according to any of statements 1 to 39, which is a method for identifying a maize plant or plant part having (increased) resistance to Northern Corn Leaf Blight (NCLB).

41. The method according to any of statements 1 to 40, which is a method for identifying a maize plant or plant part comprising a (fragment of a, preferably an RLK1 comprising fragment of a) HT2 or HT3 allele.

42. A method for generating a maize plant or plant part, preferably a maize plant having increased pathogen resistance and/or tolerance, preferably wherein said pathogen is Exserohilum turcicum, comprising introducing into the genome of a plant or a plant part a polynucleic acid as defined in any of statements 1 to 41.

43. A method for generating a maize plant or plant part, comprising introducing in the genome of a maize plant or plant part a polynucleic acid comprising a (fragment of a, preferably an RLK1 comprising fragment of a) HT2 or HT3 allele which is flanked by molecular markers PZE-108092843 and PZA-003182005, or a fragment thereof comprising the RLK1 gene and one or more of molecular marker (allele) MA0045, MA0062, MA0063, MA0070, MA0071, MA0064, and optionally PZE-108095339, and/or Affx-91328160.

44. A method for generating a maize plant or plant part, comprising introducing in the genome of a maize plant or plant part a polynucleic acid comprising a (fragment of a, preferably an RLK1 comprising fragment of a) HT2 or HT3 allele which is flanked by molecular markers PZE-108092843 and PZA-003182005, or a fragment thereof comprising the RLK1 gene and two or more of molecular marker (allele) MA0045, MA0062, MA0063, MA0070, MA0071, MA0064, and optionally PZE-108095339, and/or Affx-91328160.

45. A method for generating a maize plant or plant part, comprising introducing in the genome of a maize plant or plant part a polynucleic acid comprising a (fragment of a, preferably an RLK1 comprising fragment of a) HT2 or HT3 allele which is flanked by molecular markers PZE-108092843 and PZA-003182005, or a fragment thereof comprising the RLK1 gene and three or more of molecular marker (allele) MA0045, MA0062, MA0063, MA0070, MA0071, MA0064, and optionally PZE-108095339, and/or Affx-91328160.

46. A method for generating a maize plant or plant part, comprising introducing in the genome of a maize plant or plant part a polynucleic acid comprising a (fragment of a, preferably an RLK1 comprising fragment of a) HT2 or HT3 allele which is flanked by molecular markers PZE-108092843 and PZA-003182005, or a fragment thereof comprising the RLK1 gene and four or more of molecular marker (allele) MA0045, MA0062, MA0063, MA0070, MA0071, MA0064, and optionally PZE-108095339, and/or Affx-91328160.

47. A method for generating a maize plant or plant part, comprising introducing in the genome of a maize plant or plant part a polynucleic acid comprising a (fragment of a, preferably an RLK1 comprising fragment of a) HT2 or HT3 allele which is flanked by molecular marker (allele) PZE-108092843 and PZA-003182005, or a fragment thereof comprising the RLK1 gene and five or more of molecular markers MA0045, MA0062, MA0063, MA0070, MA0071, MA0064, and optionally PZE-108095339, and/or Affx-91328160.

48. A method for generating a maize plant or plant part, comprising introducing in the genome of a maize plant or plant part a polynucleic acid comprising a (fragment of a, preferably an RLK1 comprising fragment of a) HT2 or HT3 allele which is flanked by molecular markers PZE-108092843 and PZA-003182005, or a fragment thereof comprising the RLK1 gene and molecular marker (allele) MA0045, MA0062, MA0063, MA0070, MA0071, MA0064, and optionally PZE-108095339, and/or Affx-91328160.

49. The method according to any of statements 42 to 48, wherein said polynucleic acid does not comprise any one or more of (HT2 or HT3) molecular markers (allele) PZE-108092843, PZA-003182005, PZE-108094590, PZE-108096610, PZE-108096469, MA0043 and MA0025.

50. The method according to any of statements 42 to 49, wherein said polynucleic acid does not comprise (HT2 or HT3) molecular markers (allele) PZE-108092843 and/or PZA-003182005.

51. The method according to any of statements 42 to 49, wherein said polynucleotide does not comprise (HT2 or HT3) molecular markers (allele) PZE-108094590 and/or PZE-108096610.

52. The method according to any of statements 42 to 49, wherein said polynucleotide does not comprise (HT2 or HT3) molecular markers (allele) PZE-108094590 and/or PZE-108096469.

53. The method according to any of statements 42 to 49, wherein said polynucleotide does not comprise (HT2 or HT3) molecular markers (allele) MA0043 and/or MA0025.

54. The method according to any of statements 42 to 53, wherein introducing into the genome comprises transgenesis.

55. The method according to any of statements 42 to 53, wherein introducing into the genome comprises introgression.

56. The method according to any of statements 42 to 55, comprising transforming a plant or plant part, preferably a plant cell, more preferably a protoplast, with a polynucleic acid as defined in any of statements 1 to 41, and optionally regenerating a plant from said plant cell, preferably protoplast.

57. The method according to any of statements 42 to 56, wherein said polynucleic acid which is introduced has a different sequence than a corresponding polynucleic acid of the plant.

58. A method for generating a maize plant or plant part, preferably a maize plant or plant part having increased pathogen resistance and/or tolerance, preferably wherein said pathogen is Exserohilum turcicum, comprising (a) providing a first maize plant identified according to any of statements 1 to 40 or generated according to any of statements 41 to 45, (b) crossing said first maize plant with a second maize plant, (c) selecting progeny plants comprising said polynucleic acid, any one or more of molecular marker (allele) MA0045, MA0062, MA0063, MA0070, MA0071, MA0064, PZE-108095339, and Affx-91328160; and optionally (d) harvesting said maize plant part from said progeny.

59. The method according to any of statements 42 to 58, wherein said plant or plant part has increased resistance and/or tolerance to a pathogen.

60. The method according to statement 59, wherein said pathogen is a fungus, preferably causing Northern Corn Leaf Blight.

61. The method according to any of statements 59 or 60, wherein said pathogen is a Exserohilum sp.

62. The method according to any of statements 59 to 61, wherein said pathogen is Exserohilum turcicum.

63. The method according to any of statements 59 to 62, wherein said pathogen is Exserohilum turcicum causing NCLB.

64. The method according to any of statements 1 to 63, wherein said plant part is a cell, tissue or organ.

65. The method according to any of statements 1 to 64, wherein said plant part is a protoplast.

66. The method according to any of statements 1 to 64, wherein said plant part is a seed.

67. The method according to any of statements 1 to 66, wherein said polynucleic acid or molecular marker (allele) is homozygous.

68. The method according to any of statements 1 to 66, wherein said polynucleic acid or molecular marker (allele) is heterozygous.

69. A (isolated) polynucleic acid comprising a fragment of genomic interval of Zea mays chromosome 8 between molecular markers PZE-108092843 and PZA-003182005 and comprising one or more marker (allele) selected from MA0045, MA0062, MA0063, MA0070, MA0071, MA0064, PZE-108095339, and/or Affx-91328160, the complement or reverse complement thereof.

70. A (isolated) polynucleic acid comprising a fragment of genomic interval of Zea mays chromosome 8 between molecular markers PZE-108092843 and PZA-003182005 and comprising marker (allele) MA0045, the complement or reverse complement thereof.

71. A (isolated) polynucleic acid comprising a fragment of genomic interval of Zea mays chromosome 8 between molecular markers PZE-108092843 and PZA-003182005 and comprising marker (allele) MA0062, the complement or reverse complement thereof.

72. A (isolated) polynucleic acid comprising a fragment of genomic interval of Zea mays chromosome 8 between molecular markers PZE-108092843 and PZA-003182005 and comprising marker (allele) MA0063, the complement or reverse complement thereof.

73. A (isolated) polynucleic acid comprising a fragment of genomic interval of Zea mays chromosome 8 between molecular markers PZE-108092843 and PZA-003182005 and comprising marker (allele) MA0070, the complement or reverse complement thereof.

74. A (isolated) polynucleic acid comprising a fragment of genomic interval of Zea mays chromosome 8 between molecular markers PZE-108092843 and PZA-003182005 and comprising marker (allele) MA0071, the complement or reverse complement thereof.

75. A (isolated) polynucleic acid comprising a fragment of genomic interval of Zea mays chromosome 8 between molecular markers PZE-108092843 and PZA-003182005 and comprising marker (allele) MA0064, the complement or reverse complement thereof.

76. A (isolated) polynucleic acid comprising a fragment of genomic interval of Zea mays chromosome 8 between molecular markers PZE-108092843 and PZA-003182005 and comprising marker (allele) PZE-108095339, the complement or reverse complement thereof.

77. A (isolated) polynucleic acid comprising a fragment of genomic interval of Zea mays chromosome 8 between molecular markers PZE-108092843 and PZA-003182005 and comprising marker (allele) Affx-91328160, the complement or reverse complement thereof.

78. A (isolated) polynucleic acid comprising a molecular marker (allele) selected from MA0045, MA0062, MA0063, MA0070, MA0071, MA0064, PZE-108095339, and/or Affx-91328160, the complement, or the reverse complement thereof.

79. A (isolated) polynucleic acid comprising molecular marker (allele) MA0045, the complement, or the reverse complement thereof.

80. A (isolated) polynucleic acid comprising molecular marker (allele) MA0062, the complement, or the reverse complement thereof.

81. A (isolated) polynucleic acid comprising molecular marker (allele) MA0063, the complement, or the reverse complement thereof.

82. A (isolated) polynucleic acid comprising molecular marker (allele) MA0070, the complement, or the reverse complement thereof.

83. A (isolated) polynucleic acid comprising molecular marker (allele) MA0071, the complement, or the reverse complement thereof.

84. A (isolated) polynucleic acid comprising molecular marker (allele) MA0064, the complement, or the reverse complement thereof.

85. A (isolated) polynucleic acid comprising molecular marker (allele) PZE-108095339, the complement, or the reverse complement thereof.

86. A (isolated) polynucleic acid comprising molecular marker (allele) Affx-91328160, the complement, or the reverse complement thereof.

87. A (isolated) polynucleic acid capable of specifically hybridizing with a polynucleic acid according to any of statements 69 to 86.

88. The (isolated) polynucleotide according to any of statements 69 to 87, wherein said polynucleotide has a length ranging from 15 to 500 nucleotides, preferably 15 to 100 nucleotides, preferably 15 to 50 nucleotides, more preferably 15 to 35 nucleotides.

89. The (isolated) polynucleotide according to any of statements 69 to 88, wherein said polynucleotide is a primer or a probe.

90. The (isolated) polynucleotide according to any of statements 69 to 89, wherein said polynucleotide is an allele-specific primer or probe.

91. The (isolated) polynucleotide according to statement 90, wherein said polynucleotide is a KASP primer.

92. The (isolated) polynucleotide according to any of statements 78 to 91 comprising a fragment of genomic interval of Zea mays chromosome 8 between molecular markers PZE-108092843 and PZA-003182005 and comprising one or more marker (allele) selected from MA0045, MA0062, MA0063, MA0070, MA0071, MA0064, PZE-108095339, and/or Affx-91328160, the complement or reverse complement thereof.

93. A primer set capable of amplifying a polynucleotide comprising a molecular marker selected from MA0045, MA0062, MA0063, MA0070, MA0071, MA0064, PZE-108095339, or Affx-91328160.

94. A maize plant, preferably a maize plant having (increased) pathogen resistance and/or tolerance, preferably wherein said pathogen is Exserohilum turcicum obtainable by the method according to any of statements 42 to 68, or a plant part thereof, or the progeny thereof.

95. A maize plant, preferably a maize plant having increased pathogen resistance and/or tolerance, preferably wherein said pathogen is Exserohilum turcicum comprising a polynucleic acid or one or more molecular marker (allele) as defined in any of statements 1 to 41.

96. A maize plant or plant part comprising any one or more of molecular marker alleles MA0045, MA0062, MA0070, MA0071, optionally MA0063 and MA0064, wherein
MA0045 is a SNP which is A at a position corresponding to position 156,789,751 bp of chromosome 8 of the B73 reference genome AGPv4;
MA0062 is a SNP which is G at a position corresponding to position 156,772,431 bp of chromosome 8 of the B73 reference genome AGPv4;
MA0063 is a SNP which is T at a position corresponding to position 156,543,061 bp of chromosome 8 of the B73 reference genome AGPv4;
MA0070 is a SNP which is T at a position corresponding to position 156,694,565 bp of chromosome 8 of the B73 reference genome AGPv4;
MA0071 is a SNP which is C at a position corresponding to position 156,694,641 bp of chromosome 8 of the B73 reference genome AGPv4;
MA0064 is a SNP which is C at a position corresponding to position 156,541,890 bp of chromosome 8 of the B73 reference genome AGPv4; and optionally
MA0021, which is a SNP which is G at a position corresponding to position 156,535,845 bp of chromosome 8 of the B73 reference genome AGPv4; and/or
MA0035, which is a SNP which is A at a position corresponding to position 156,542,253 bp of chromosome 8 of the B73 reference genome AGPv4.

97. A maize plant or plant part comprising any two or more of molecular marker alleles MA0045, MA0062, MA0063, and MA0064, wherein
MA0045 is a SNP which is A at a position corresponding to position 156,789,751 bp of chromosome 8 of the B73 reference genome AGPv4;
MA0062 is a SNP which is G at a position corresponding to position 156,772,431 bp of chromosome 8 of the B73 reference genome AGPv4;
MA0063 is a SNP which is T at a position corresponding to position 156,543,061 bp of chromosome 8 of the B73 reference genome AGPv4;
MA0070 is a SNP which is T at a position corresponding to position 156,694,565 bp of chromosome 8 of the B73 reference genome AGPv4;
MA0071 is a SNP which is C at a position corresponding to position 156,694,641 bp of chromosome 8 of the B73 reference genome AGPv4;
MA0064 is a SNP which is C at a position corresponding to position 156,541,890 bp of chromosome 8 of the B73 reference genome AGPv4.

98. A maize plant or plant part comprising any three or more, preferably any four or more, more preferably any five or more, most preferably all of molecular marker alleles MA0045, MA0062, MA0063, MA0070, MA0071, and MA0064, wherein
MA0045 is a SNP which is A at a position corresponding to position 156,789,751 bp of chromosome 8 of the B73 reference genome AGPv4;
MA0062 is a SNP which is G at a position corresponding to position 156,772,431 bp of chromosome 8 of the B73 reference genome AGPv4;
MA0063 is a SNP which is T at a position corresponding to position 156,543,061 bp of chromosome 8 of the B73 reference genome AGPv4;
MA0070 is a SNP which is T at a position corresponding to position 156,694,565 bp of chromosome 8 of the B73 reference genome AGPv4;
MA0071 is a SNP which is C at a position corresponding to position 156,694,641 bp of chromosome 8 of the B73 reference genome AGPv4;
MA0064 is a SNP which is C at a position corresponding to position 156,541,890 bp of chromosome 8 of the B73 reference genome AGPv4.

99. A maize plant or plant part comprising molecular marker alleles MA0045, MA0062, MA0063, MA0070, MA0071, and MA0064, wherein
MA0045 is a SNP which is A at a position corresponding to position 156,789,751 bp of chromosome 8 of the B73 reference genome AGPv4;
MA0062 is a SNP which is G at a position corresponding to position 156,772,431 bp of chromosome 8 of the B73 reference genome AGPv4;
MA0063 is a SNP which is T at a position corresponding to position 156,543,061 bp of chromosome 8 of the B73 reference genome AGPv4;
MA0070 is a SNP which is T at a position corresponding to position 156,694,565 bp of chromosome 8 of the B73 reference genome AGPv4;
MA0071 is a SNP which is C at a position corresponding to position 156,694,641 bp of chromosome 8 of the B73 reference genome AGPv4;
MA0064 is a SNP which is C at a position corresponding to position 156,541,890 bp of chromosome 8 of the B73 reference genome AGPv4.

100. The maize plant or plant part according to any of statements 96 to 99, further comprising molecular marker allele PZE-108095339, wherein PZE-108095339 is a SNP which is G at a position corresponding to position 156,378,424 bp of chromosome 8 of the B73 reference genome AGPv4.

101. The maize plant or plant part according to any of statements 96 to 100, further comprising molecular marker allele Affx-91328160, wherein Affx-91328160 is a SNP which is G at a position corresponding to position 156,372,823 bp of chromosome 8 of the B73 reference genome AGPv4.

102. The maize plant or plant part according to any of statements 96 to 101, wherein said plant or plant part does not comprise (HT2 or HT3) molecular marker allele PZE-108092843 and/or PZA-003182005, wherein
PZE-108092843, which is a SNP which is C at a position corresponding to position 154,456,106 bp of chromosome 8 of the B73 reference genome AGPv4;
PZA-003182005, which is a SNP which is A at a position corresponding to position 158,167,578 bp of chromosome 8 of the B73 reference genome AGPv4.

103. The maize plant or plant part according to any of statements 96 to 102, wherein said plant or plant part does not comprise (HT2 or HT3) molecular marker allele PZE-108094590 and/or PZE-108096610, wherein
PZE-108094590, which is a SNP which is G at a position corresponding to position 155,860,812 bp of chromosome 8 of the B73 reference genome AGPv4;
PZE-108096610, which is a SNP which is G at a position corresponding to position 157,375,673 bp of chromosome 8 of the B73 reference genome AGPv4.

104. The maize plant or plant part according to any of statements 96 to 103, wherein said plant or plant part does not comprise (HT2 or HT3) molecular marker allele PZE-108094590 and/or PZE-108096469, wherein
PZE-108094590, which is a SNP which is G at a position corresponding to position 155,860,812 bp of chromosome 8 of the B73 reference genome AGPv4;
PZE-108096469, which is a SNP which is G at a position corresponding to position 157,266,475 bp of chromosome 8 of the B73 reference genome AGPv4.

105. The maize plant or plant part according to any of statements 96 to 104, wherein said plant or plant part does not comprise (HT2 or HT3) molecular marker allele MA0043 and/or MA0025, wherein
MA0043, which is a SNP which is A at a position corresponding to position 155,999,733 bp of chromosome 8 of the B73 reference genome AGPv4;
MA0025, which is a SNP which is G at a position corresponding to position 156,967,599 bp of chromosome 8 of the B73 reference genome AGPv4.

106. The maize plant or plant part according to any of statements 94 to 105, which is transgenic, gene-edited, or mutagenized.

107. A method for identifying a maize plant or plant part, comprising:
crossing a first maize plant according to any of statements 94 to 106 with a second maize plant
identifying an offspring maize plant or plant part with the method according to any of statements 1 to 41.

108. Method for controlling pathogen infestation in a maize plant (population) comprising
a) Providing (a) maize plant(s) according to any of statements 94 to 106 or growing from seeds (a) maize plant(s) according to any of statements 94 to 106,
b) Cultivating the plant(s) of a) under conditions of pathogen infestation.

109. The method according to statement 108, wherein pathogen infestation is reduced.

110. The method according to statement 108 or 109, wherein pathogen symptoms are reduced.

111. The method according to any of statements 108 to 110, wherein said conditions of pathogen infestation comprise the presence of a pathogen.

112. The method according to any of statements 108 to 111, wherein said pathogen is a fungus.

113. The method according to any of statements 108 to 112, wherein said pathogen is a Exserohilum sp.

114. The method according to any of statements 108 to 113, wherein said pathogen is Exserohilum turcicum.

115. The method according to any of statements 108 to 114, wherein said pathogen is Exserohilum turcicum causing NCLB.

116. Use of a polynucleic acid according to any of statements 1 to 41 or 69 to 87 for generating a maize plant or plant part.

117. Use of a polynucleic acid according to statement 116 for generating a maize plant having (increased) pathogen resistance and/or tolerance or for conferring or increasing pathogen resistance and/or tolerance in a maize plant or plant part.

118. Use of a polynucleic acid according to any of statements 1 to 41 or 69 to 92 or the primer pair according to statement 93 for identifying a maize plant or plant part.

119. Use according to statement 118 for identifying a maize plant or plant part having (increased) pathogen resistance and/or tolerance.

120. Use of a maize plant according to any of statements 94 to 106 for controlling pathogen infestation in a maize plant (population).

121. Use of a maize plant according to any of statements 94 to 106 for increasing the yield (potential) of a plant, preferably under conditions of pathogen infestation.

122. Use according to statement 121, wherein the yield is biomass or seed yield.

123. Use according to statement 122, wherein said biomass is whole plant biomass or biomass of a plant part.

124. Use according to statement 123, wherein said plant part is a tissue, organ, fruit, or seed.

125. Use according to any of statements 116 to 124, wherein said maize plant or plant part has (increased) resistance to NCLB.

126. Use according to any of statements 116 to 125, wherein said maize plant or plant part has (increased) resistance to Exserohilum sp.

127. Use according to any of statements 116 to 126, wherein said maize plant or plant part has (increased) resistance to Exserohilum turcicum.

128. Use according to any of statements 116 to 127, wherein said maize plant or plant part has (increased) resistance to Exserohilum turcicum causing NCLB.

129. The (isolated) polynucleotide according to any of statements 69 to 92, comprising, consisting essentially of, or consisting of a sequence of any of SEQ ID NOs: 4-51 or fragments thereof, preferably fragments comprising at least the 15 most 3′ contiguous nucleotides thereof, the complement or reverse complement thereof.

130. The (isolated) polynucleotide according to any of statements 69 to 92 comprising, consisting essentially of, or consisting of a sequence of SEQ ID NO: 4 or 22 or fragments thereof, preferably fragments comprising at least the 15 most 3′ contiguous nucleotides thereof, the complement or reverse complement thereof.

131. The (isolated) polynucleotide according to any of statements 69 to 92, comprising, consisting essentially of, or consisting of a sequence of SEQ ID NO: 5 or 23 or fragments thereof, preferably fragments comprising at least the 15 most 3′ contiguous nucleotides thereof, the complement or reverse complement thereof.

132. The (isolated) polynucleotide according to any of statements 69 to 92, comprising, consisting essentially of, or consisting of a sequence of SEQ ID NO: 7 or 25 or fragments thereof, preferably fragments comprising at least the 15 most 3′ contiguous nucleotides thereof, the complement or reverse complement thereof.

133. The (isolated) polynucleotide according to any of statements 69 to 92, comprising, consisting essentially of, or consisting of a sequence of SEQ ID NO: 8 or 26 or fragments thereof, preferably fragments comprising at least the 15 most 3′ contiguous nucleotides thereof, the complement or reverse complement thereof.

134. The (isolated) polynucleotide according to any of statements 69 to 92, comprising, consisting essentially of, or consisting of a sequence of SEQ ID NO: 10 or 28 or fragments thereof, preferably fragments comprising at least the 15 most 3′ contiguous nucleotides thereof, the complement or reverse complement thereof.

135. The (isolated) polynucleotide according to any of statements 69 to 92, comprising, consisting essentially of, or consisting of a sequence of SEQ ID NO: 11 or 29 or fragments thereof, preferably fragments comprising at least the 15 most 3′ contiguous nucleotides thereof, the complement or reverse complement thereof.

136. The (isolated) polynucleotide according to any of statements 69 to 92, comprising, consisting essentially of, or consisting of a sequence of SEQ ID NO: 13 or 31 or fragments thereof, preferably fragments comprising at least the 15 most 3′ contiguous nucleotides thereof, the complement or reverse complement thereof.

137. The (isolated) polynucleotide according to any of statements 69 to 92, comprising, consisting essentially of, or consisting of a sequence of SEQ ID NO: 14 or 32 or fragments thereof, preferably fragments comprising at least the 15 most 3′ contiguous nucleotides thereof, the complement or reverse complement thereof.

138. The (isolated) polynucleotide according to any of statements 69 to 92, comprising, consisting essentially of, or consisting of a sequence of SEQ ID NO: 16 or 34 or fragments thereof, preferably fragments comprising at least the 15 most 3′ contiguous nucleotides thereof, the complement or reverse complement thereof.

139. The (isolated) polynucleotide according to any of statements 69 to 92, comprising, consisting essentially of, or consisting of a sequence of SEQ ID NO: 17 or 35 or fragments thereof, preferably fragments comprising at least the 15 most 3′ contiguous nucleotides thereof, the complement or reverse complement thereof.

140. The (isolated) polynucleotide according to any of statements 69 to 92, comprising, consisting essentially of, or consisting of a sequence of SEQ ID NO: 19 or 37 or fragments thereof, preferably fragments comprising at least the 15 most 3′ contiguous nucleotides thereof, the complement or reverse complement thereof.

141. The (isolated) polynucleotide according to any of statements 69 to 92, comprising, consisting essentially of, or consisting of a sequence of SEQ ID NO: 20 or 38 or fragments thereof, preferably fragments comprising at least the 15 most 3′ contiguous nucleotides thereof, the complement or reverse complement thereof.

142. The (isolated) polynucleotide according to any of statements 69 to 92, comprising, consisting essentially of, or consisting of a sequence of SEQ ID NO: 40 or 46 or fragments thereof, preferably fragments comprising at least the 15 most 3′ contiguous nucleotides thereof, the complement or reverse complement thereof.

143. The (isolated) polynucleotide according to any of statements 69 to 92, comprising, consisting essentially of, or consisting of a sequence of SEQ ID NO: 41 or 47 or fragments thereof, preferably fragments comprising at least the 15 most 3′ contiguous nucleotides thereof, the complement or reverse complement thereof.

144. The (isolated) polynucleotide according to any of statements 69 to 92, comprising, consisting essentially of, or consisting of a sequence of SEQ ID NO: 43 or 49 or fragments thereof, preferably fragments comprising at least the 15 most 3′ contiguous nucleotides thereof, the complement or reverse complement thereof.

145. The (isolated) polynucleotide according to any of statements 69 to 92, comprising, consisting essentially of, or consisting of a sequence of SEQ ID NO: 44 or 50 or fragments thereof, preferably fragments comprising at least the 15 most 3′ contiguous nucleotides thereof, the complement or reverse complement thereof.

146. The primer set according to statement 93, comprising a primer comprising, consisting essentially of, or consisting of a sequence of SEQ ID NO: 4 or 22 and a primer comprising, consisting essentially of, or consisting of a sequence of SEQ ID NO: 5 or 23, preferably SEQ ID NO: 4 and 5 or SEQ ID NO: 22 and 23, optionally further comprising a primer comprising, consisting essentially of, or consisting of a sequence of SEQ ID NO: 6; or fragments thereof, preferably fragments comprising at least the 15 most 3′ contiguous nucleotides thereof, the complement or reverse complement thereof.

147. The primer set according to statement 93, comprising a primer comprising, consisting essentially of, or consisting of a sequence of SEQ ID NO: 7 or 25 and a primer comprising, consisting essentially of, or consisting of a sequence of SEQ ID NO: 8 or 26, preferably SEQ ID NO: 7 and 8 or SEQ ID NO: 25 and 26, optionally further comprising a primer comprising, consisting essentially of, or consisting of a sequence of SEQ ID NO: 9; or fragments thereof, preferably fragments comprising at least the 15 most 3′ contiguous nucleotides thereof, the complement or reverse complement thereof.

148. The primer set according to statement 93, comprising a primer comprising, consisting essentially of, or consisting of a sequence of SEQ ID NO: 10 or 28 and a primer comprising, consisting essentially of, or consisting of a sequence of SEQ ID NO: 11 or 29, preferably SEQ ID NO: 10 and 11 or SEQ ID NO: 28 and 29, optionally further comprising a primer comprising, consisting essentially of, or consisting of a sequence of SEQ ID NO: 12; or fragments thereof, preferably fragments comprising at least the 15 most 3′ contiguous nucleotides thereof, the complement or reverse complement thereof.

149. The primer set according to statement 93, comprising a primer comprising, consisting essentially of, or consisting of a sequence of SEQ ID NO: 13 or 31 and a primer comprising, consisting essentially of, or consisting of a sequence of SEQ ID NO: 14 or 32, preferably SEQ ID NO: 13 and 14 or SEQ ID NO: 31 and 32, optionally further comprising a primer comprising, consisting essentially of, or consisting of a sequence of SEQ ID NO: 15; or fragments thereof, preferably fragments comprising at least the 15 most 3′ contiguous nucleotides thereof, the complement or reverse complement thereof.

150. The primer set according to statement 93, comprising a primer comprising, consisting essentially of, or consisting of a sequence of SEQ ID NO: 16 or 34 and a primer comprising, consisting essentially of, or consisting of a sequence of SEQ ID NO: 17 or 35, preferably SEQ ID NO: 16 and 17 or SEQ ID NO: 34 and 35, optionally further comprising a primer comprising, consisting essentially of, or consisting of a sequence of SEQ ID NO: 18; or fragments thereof, preferably fragments comprising at least the 15 most 3′ contiguous nucleotides thereof, the complement or reverse complement thereof.

151. The primer set according to statement 93, comprising a primer comprising, consisting essentially of, or consisting of a sequence of SEQ ID NO: 19 or 37 and a primer comprising, consisting essentially of, or consisting of a sequence of SEQ ID NO: 20 or 38, preferably SEQ ID NO: 19 and 20 or SEQ ID NO: 37 and 38, optionally further comprising a primer comprising, consisting essentially of, or consisting of a sequence of SEQ ID NO: 21; or fragments thereof, preferably fragments comprising at least the 15 most 3′ contiguous nucleotides thereof, the complement or reverse complement thereof.

152. The primer set according to statement 93, comprising a primer comprising, consisting essentially of, or consisting of a sequence of SEQ ID NO: 40 or 46 and a primer comprising, consisting essentially of, or consisting of a sequence of SEQ ID NO: 41 or 47, preferably SEQ ID NO: 40 and 41 or SEQ ID NO: 46 and 47, optionally further comprising a primer comprising, consisting essentially of, or consisting of a sequence of SEQ ID NO: 42; or fragments thereof, preferably fragments comprising at least the 15 most 3′ contiguous nucleotides thereof, the complement or reverse complement thereof.

153. The primer set according to statement 93, comprising a primer comprising, consisting essentially of, or consisting of a sequence of SEQ ID NO: 43 or 49 and a primer comprising, consisting essentially of, or consisting of a sequence of SEQ ID NO: 44 or 50, preferably SEQ ID NO: 43 and 44 or SEQ ID NO: 49 and 50, optionally further comprising a primer comprising, consisting essentially of, or consisting of a sequence of SEQ ID NO: 45; or fragments thereof, preferably fragments comprising at least the 15 most 3′ contiguous nucleotides thereof, the complement or reverse complement thereof.

154. The (isolated) polynucleotide according to any of statements 69 to 92, comprising, consisting essentially of, or consisting of a sequence of any of SEQ ID NOs: 52 to 59 or (unique) fragments thereof, preferably of at least 15, more preferably at least 18 contiguous nucleotides, the complement or reverse complement thereof or of the fragment.

155. The isolated polynucleotide according to statement 154, comprising at least
R at position 21 of SEQ ID NO: 52, preferably as the most 3′ or 5′, 2nd most 3′ or 5′, or 3rd most 3′ or 5′ nucleotide;
R at position 21 of SEQ ID NO: 53, preferably as the most 3′ or 5′, 2nd most 3′ or 5′, or 3rd most 3′ or 5′ nucleotide;
Y at position 21 of SEQ ID NO: 54, preferably as the most 3′ or 5′, 2nd most 3′ or 5′, or 3rd most 3′ or 5′ nucleotide;
Y at position 21 of SEQ ID NO: 55, preferably as the most 3′ or 5′, 2nd most 3′ or 5′, or 3rd most 3′ or 5′ nucleotide;
K at position 21 of SEQ ID NO: 56, preferably as the most 3′ or 5′, 2nd most 3′ or 5′, or 3rd most 3′ or 5′ nucleotide;
M at position 21 of SEQ ID NO: 57, preferably as the most 3′ or 5′, 2nd most 3′ or 5′, or 3rd most 3′ or 5′ nucleotide;
K at position 31 of SEQ ID NO: 58, preferably as the most 3′ or 5′, 2nd most 3′ or 5′, or 3rd most 3′ or 5′ nucleotide; or
Y at position 31 of SEQ ID NO: 59, preferably as the most 3′ or 5′, 2nd most 3′ or 5′, or 3rd most 3′ or 5′ nucleotide.

156. A (isolated) polynucleotide comprising consisting essentially of, or consisting of a sequence of any of SEQ ID NO: 52-59, or a (unique) fragment thereof, preferably of at least 15, more preferably at least 18 contiguous nucleotides, the complement or the reverse complement thereof.

157. The isolated polynucleotide according to statement 156, comprising at least
R at position 21 of SEQ ID NO: 52, preferably as the most 3′ or 5′, 2nd most 3′ or 5′, or 3rd most 3′ or 5′ nucleotide;
R at position 21 of SEQ ID NO: 53, preferably as the most 3′ or 5′, 2nd most 3′ or 5′, or 3rd most 3′ or 5′ nucleotide;
Y at position 21 of SEQ ID NO: 54, preferably as the most 3′ or 5′, 2nd most 3′ or 5′, or 3rd most 3′ or 5′ nucleotide;
Y at position 21 of SEQ ID NO: 55, preferably as the most 3′ or 5′, 2nd most 3′ or 5′, or 3rd most 3′ or 5′ nucleotide;
K at position 21 of SEQ ID NO: 56, preferably as the most 3′ or 5′, 2nd most 3′ or 5′, or 3rd most 3′ or 5′ nucleotide;
M at position 21 of SEQ ID NO: 57, preferably as the most 3′ or 5′, 2nd most 3′ or 5′, or 3rd most 3′ or 5′ nucleotide;
K at position 31 of SEQ ID NO: 58, preferably as the most 3′ or 5′, 2nd most 3′ or 5′, or 3rd most 3′ or 5′ nucleotide; or
Y at position 31 of SEQ ID NO: 59, preferably as the most 3′ or 5′, 2nd most 3′ or 5′, or 3rd most 3′ or 5′ nucleotide.

### BRIEF DESCRIPTION OF THE FIGURES

FIG. 1: Physical map of marker positions with reference to the marker positions of AGPv04.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any or etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

In an aspect, the invention relates to a method for identifying a maize plant or plant part, comprising screening for the presence of or detecting or identifying a polynucleic acid comprising any one or more, two or more, three or more, or all of molecular markers or (HT2/HT3) molecular marker alleles selected from MA0045, MA0062, MA0063, MA0070, MA0071, and MA0064, optionally additionally molecular marker (allele) PZE-108095339 and/or Affx-91328160. Particular suitable combinations of marker alleles are described herein elsewhere.

In an aspect, the invention relates to a method for identifying a maize plant or plant part, comprising screening for the presence of or detecting or identifying any one or more, two or more, three or more, or all of molecular markers or (HT2/HT3) molecular marker alleles selected from MA0045, MA0062, MA0063, MA0070, MA0071, and MA0064, optionally molecular marker (allele) PZE-108095339 and/or Affx-91328160. Particular suitable combinations of marker alleles are described herein elsewhere.

The combination of any one or more of the above marker(s) (allele(s)) may be referred to as a marker haplotype of the invention.

The methods may comprise screening of a sample obtained from a maize plant or plant part, in particular a sample comprising genomic DNA of the maize plant or plant part. Accordingly, the method may comprise the step of obtaining a sample (comprising genomic DNA) from a maize plant or plant part, or providing a sample (comprising genomic DNA) obtained from a maize plant or plant part. Methods for screening or identifying markers are well known in the art, as also described herein elsewhere.

The molecular marker(s) (allele(s)) are present in the HT2 and HT3 locus, and are located on Zea mays chromosome 8. The locus comprises a genomic interval ranging from molecular marker (allele) PZE-108092843 to PZA-003182005. While the locus may comprise a larger genomic fragment, markers PZE-108092843 to PZA-003182005 delineate a suitable smaller fragment responsible for Exserohilum turcicum resistance. Accordingly, the polynucleic acid as described herein according to the invention in certain embodiments corresponds to (i.e. has a sequence of) a Zea mays chromosome 8 interval flanked by (and including or alternatively excluding) molecular marker (allele) PZE-108092843 and PZA-003182005. In certain embodiments, the polynucleic acid as described herein according to the invention corresponds to (i.e. has a sequence of) a Zea mays chromosome 8 interval flanked by (and including or alternatively excluding) molecular marker (allele) PZE-108094590 and PZE-108096610. In certain embodiments, the polynucleic acid as described herein according to the invention corresponds to (i.e. has a sequence of) a Zea mays chromosome 8 interval flanked by (and including or alternatively excluding) molecular marker (allele) PZE-108094590 and PZE-108096469. In certain embodiments, the polynucleic acid as described herein according to the invention corresponds to (i.e. has a sequence of) a Zea mays chromosome 8 interval flanked by (and including or alternatively excluding) molecular marker (allele) MA0043 and MA0025. In certain embodiments, the polynucleic acid as described herein according to the invention corresponds to (i.e. has a sequence of) a Zea mays chromosome 8 interval flanked by (and including or alternatively excluding) molecular marker (allele) PZE-108092843, PZE-108094590, or MA0043 and molecular marker (allele) PZA-003182005, PZE-108096610, PZE-108096469, or MA0025. In certain embodiments, the polynucleic acid as described herein according to the invention corresponds to (i.e. has a sequence of) a Zea mays chromosome 8 interval flanked by (and including or alternatively excluding) molecular marker (allele) PZE-108092843 and PZE-108096610. In certain embodiments, the polynucleic acid as described herein according to the invention corresponds to (i.e. has a sequence of) a Zea mays chromosome 8 interval flanked by (and including or alternatively excluding) molecular marker (allele) PZE-108092843 and PZE-108096469. In certain embodiments, the polynucleic acid as described herein according to the invention corresponds to (i.e. has a sequence of) a Zea mays chromosome 8 interval flanked by (and including or alternatively excluding) molecular marker (allele) PZE-108092843 and MA0025. In certain embodiments, the polynucleic acid as described herein according to the invention corresponds to (i.e. has a sequence of) a Zea mays chromosome 8 interval flanked by (and including or alternatively excluding) molecular marker (allele) PZE-108094590 and PZA-003182005. In certain embodiments, the polynucleic acid as described herein according to the invention corresponds to (i.e. has a sequence of) a Zea mays chromosome 8 interval flanked by (and including or alternatively excluding) molecular marker (allele) PZE-108094590 and MA0025. In certain embodiments, the polynucleic acid as described herein according to the invention corresponds to (i.e. has a sequence of) a Zea mays chromosome 8 interval flanked by (and including or alternatively excluding) molecular marker (allele) MA0043 and PZA-003182005. In certain embodiments, the polynucleic acid as described herein according to the invention corresponds to (i.e. has a sequence of) a Zea mays chromosome 8 interval flanked by (and including or alternatively excluding) molecular marker (allele) MA0043 and PZE-108096610. In certain embodiments, the polynucleic acid as described herein according to the invention corresponds to (i.e. has a sequence of) a Zea mays chromosome 8 interval flanked by (and including or alternatively excluding) molecular marker (allele) MA0043 and PZE-108096469. Suitable polynucleic acids according to the invention (or maize plants or plant parts according to the invention) can be identified for instance by the presence of one or more of (HT2 or HT3) marker (allele) MA0045, MA0062, MA0063, MA0070, MA0071, and MA0064, and optionally molecular marker (allele) PZE-108095339 and/or Affx-91328160 and by the absence of one or more of (HT2 or HT3) molecular marker (allele) PZE-108092843, PZA-003182005, PZE-108094590, PZE-108096610, PZE-108096469, MA0043 and MA0025.

The molecular marker(s) (allele(s)) of the present invention can be advantageously used to identify maize plants or plant parts, in particular maize plants or plant part having resistance or tolerance or having increased resistance or tolerance to pathogens, in particular fungal pathogens, such as Exserohilum turcicum.

The molecular marker(s) (allele(s)) of the present invention can be advantageously used to identify maize plants or plant parts comprising the HT2 locus as well as the HT3 locus, and in particular the HT2/HT3 allele of RLK1. The HT2 and HT3 allele of RLK1 is for instance described in WO2019/038326, which is incorporated by reference herein in its entirety.

Each of these diagnostic markers can be used to follow a small donor introgression of HT2 or HT3 in breeding processes, since each marker is diagnostic and closely linked to the causative gene conferring HT2/HT3 resistance. The molecular marker(s) (allele(s)) of the present invention are closely linked to the RLK1 gene of the HT2 locus and the HT3 locus and can therefore be advantageously used to identify maize plants or plant parts comprising a minimal fragment of the HT2/HT3 allele of RLK1 wherein the maize plant or plant part does not comprise potentially detrimental flanking sequences which negatively affect agronomically relevant characteristics, such as for instance yield potential. Accordingly, the molecular marker(s) (allele(s)) of the present invention are suitable for marker-assisted breeding, in particular in order to avoid or minimize linkage drag. The markers allow more efficient introgression because very favourable recombination can be easily detected. Indeed, in order to actively select against linkage drag, suitable markers need to be in close proximity to a trait of interest, such that recombination between a trait of interest (e.g. pathogen resistance) and its flanking regions can be adequately monitored.

From WO2015/032494 (incorporated herein by reference in its entirety) which investigated and used introgression lines with Htn1 from Pepitilla (of which the causative gene is also RLK1), it is known that this resistance locus is closely linked to genomic regions carrying linkage drag resulting in negative effects on one or more agronomic features. First investigation of the flanking region of HT2 (or HT3) indicated that this or similar linkage drag is not only present in Pepitilla donor for HtN1 introgression, but also in other donors for this Helminthosporium resistance locus like A619. Inter alia the linkage drag as part of the HT2 (or HT3) introgression can effect a difference in the flowering time, which is an important agronomic characteristic. It can directly and substantially influence the yield potential of a Zea mays plant. A delayed flowering time usually results in a reduced yield. Further linkage drag affecting the yield potential, in particular the silage yield potential, may be found distal and/or proximal of the Helminthosporium resistance locus on bin 8.06 in Zea mays. Flanking regions, closely linked to this resistance locus, might be carrier of the known linkage drag. The molecular marker(s) (allele(s)) of the present invention flank a minimal region of the HT2 or HT3 locus comprising the RLK1 gene, and can be suitably used for selection purposes as well as for delineating a minimal HT2 and/or HT3 locus for generating maize plants or plant parts, in which linkage drag is avoided or minimized. As an example, removal of linkage drag may be carried out by genetic recombination during a crossing process between two maize plants, wherein one parent maize plant carries the HT2-resistance locus or HT3-resistance locus. In addition to the use of conventional breeding techniques to produce a genetic recombination which has the result of replacing at least one of the donor intervals with linkage drag identified above with genomic sequences of the recurrent parent which are preferably free from unwanted genes, modern biotechnology offers the person skilled in the art many tools which can enable precise genetic engineering to be carried out. Examples of known tools are meganucleases (Silva et al., 2011), homing endonucleases (Chevalier 2002), zinc finger nucleases, TALE nucleases (WO 2010/079430; WO 2011/072246) or CRISPR systems (Gaj et al., 2013).

In addition to the above advantage of close linkage to RLK1, the molecular marker(s) (allele(s)) of the present invention the screening-process for improved resistance for Northern Corn Leaf Blight (NCLB) is now simplified, and genetic material could be screened for presence of a minimal HT2 or HT3 allele and introgressed with a small donor segment into material via marker-assisted selection (MAS). The molecular marker(s) (allele(s)) of the present invention allow for a robust selection procedure by their suitable uniqueness and capability to distinguish resistance and non-resistance alleles.

The location of the molecular marker(s) (allele(s)) described herein is provided in the Table below, referenced to chromosome 8 of the Zea mays B73 reference genome AGPv04 (https://www.maizegdb.org/genome/genome_assembly/Zm-B73-REFERENCE-GRAM ENE-4.0). AGPv04 is used herein interchangeably with AGPv4, also referred to a “Maize B73 RefGen_v4”. All markers are SNPs. Also indicated is the nucleotide present in the HT2 and HT3 allele (i.e. the resistance conferring allele), as well as a possible nucleotide of an alternative, non-HT2/HT3 allele (i.e. an allele not conferring resistance). It will be understood that the methods as described herein for identifying a maize plant having (increased) resistance or tolerance to a pathogen, such as Helminthosporium turcicum, involve identification of the HT2 or HT3 molecular marker alleles.

Table: Overview of HT2/HT3 markers on introgression fragment. Column ‘HT2/HT3 primer’ shows the HT2/HT3 allele (i.e. the donor allele) which is called by respective KASP primer. ‘Alternative allele” provides the alleles (SNPs) in non-HT2/HT3 sources. Column ‘Alternative primer’ shows the alternative allele (i.e. the acceptor or non HT2/HT3 allele) which is called by respective KASP primer. It should be noted that the called allele can be also located on the reverse (i.e. opposite/complementary strand) strand of the genomic DNA as available in the database (i.e. AGPv04; https://www.maizegdb.org/genome/genome_assembly/Zm-B73-REFERENCE-GRAMENE-4.0). For instance, in certain embodiments, for instance for marker Affx-91328160, the “Alternative” primer for detecting the alternative allele (preferably an allele-specific primer, such as a KASP primer) is designed to comprise nucleotide “T” corresponding to position 156372823, and hence bind (i.e. to be complementary with) the corresponding genomic DNA strand as listed in the reference genome AGPv04 comprising nucleotide “A”. Similarly, the “HT2/HT3” primer for detecting the donor allele (preferably an allele-specific primer, such as a KASP primer) is designed to comprise nucleotide “C” corresponding to position 156372823, and hence bind (i.e. to be complementary with) the corresponding genomic DNA strand as listed in the reference genome AGPv04 comprising nucleotide “G”. On the other hand, for instance for marker PZE-108095339, the “Alternative” primer for detecting the alternative allele (preferably an allele-specific primer, such as a KASP primer) is designed to comprise nucleotide “A” corresponding to position 156378424, and hence bind (i.e. to be complementary with) the complement of corresponding genomic DNA strand (i.e. the opposite strand) as listed in the reference genome AGPv04 comprising nucleotide “T” (in the opposite strand). Similarly, the “HT2/HT3” primer for detecting the donor allele (preferably an allele-specific primer, such as a KASP primer) is designed to comprise nucleotide “G” corresponding to position 156378424, and hence bind (i.e. to be complementary with) the complement of corresponding genomic DNA strand as listed in the reference genome AGPv04 comprising nucleotide “C” (in the opposite strand).

| | position | HT2/HT3- | HT2/HT3- | Alternative | Alternative |
|---|---|---|---|---|---|
| marker name | AGPv04 | allele | primer | allele | primer |
| PZE-108092843 | 154456106 | C | C | A | A |
| PZE-108094590 | 155860812 | G | G | A | A |
| MA0043 | 155999733 | A | T | G | C |
| Affx-91328160 | 156372823 | G | G | A | A |
| PZE-108095325 | 156374083 | G | G | A | A |
| PZE-108095339 | 156378424 | G | C | A | T |
| MA0021 | 156535845 | G | G | C | C |
| MA0064 | 156541890 | C | C | T | T |
| MA0035 | 156542253 | A | T | G | C |
| MA0063 | 156543061 | T | A | C | G |
| MA0070 | 156694565 | T | T | G | G |
| MA0071 | 156694641 | C | G | T | A |
| MA0062 | 156772431 | G | G | T | T |
| MA0045 | 156789751 | A | A | C | C |
| MA0025 | 156967599 | G | G | A | A |
| PZE-108096469 | 157266475 | G | C | A | T |
| PZE-108096610 | 157375673 | G | C | A | |
| PZA-003182005 | 158167578 | A | A | G | |

It is to be understood that the indicated nucleotide positions are the nucleotide positions of the indicated AGPv04 B73 chromosome 8 positions and that the marker positions in the maize plants according to the invention correspond to the indicated marker positions, but are or comprise not necessarily identical positions in a different genome (e.g. from a different race or line). The skilled person will understand that corresponding nucleotide positions can be determined by suitable alignment, as is known in the art.

In certain embodiments, the invention relates to a method for identifying a maize plant or plant part, such as a maize plant or plant part having (increased) pathogen resistance, as described herein elsewhere, comprising screening for the presence of or identifying or detecting one or more molecular marker (allele) as described herein elsewhere, wherein, a plant or plant part is identified (as having (increased) pathogen resistance) when one or more of the following marker alleles is identified or detected:
MA0045, which is a SNP which is A at a position corresponding to position 156,789,751 bp of chromosome 8 of the B73 reference genome AGPv4;
MA0062, which is a SNP which is G at a position corresponding to position 156,772,431 bp of chromosome 8 of the B73 reference genome AGPv4;
MA0063, which is a SNP which is T at a position corresponding to position 156,543,061 bp of chromosome 8 of the B73 reference genome AGPv4;
MA0070, which is a SNP which is T at a position corresponding to position 156,694,565 bp of chromosome 8 of the B73 reference genome AGPv4;
MA0071, which is a SNP which is C at a position corresponding to position 156,694,641 bp of chromosome 8 of the B73 reference genome AGPv4;
MA0064, which is a SNP which is C at a position corresponding to position 156,541,890 bp of chromosome 8 of the B73 reference genome AGPv4; and optionally
Affx-91328160, which is a SNP which is G at a position corresponding to position 156,372,823 bp of chromosome 8 of the B73 reference genome AGPv4;
PZE-108095339, which is a SNP which is G at a position corresponding to position 156,378,424 bp of chromosome 8 of the B73 reference genome AGPv4.

In certain embodiments, the maize plant does not have any one or more of molecular marker (allele):
PZE-108092843, which is a SNP which is C at a position corresponding to position 154,456,106 bp of chromosome 8 of the B73 reference genome AGPv4;
PZE-108094590, which is a SNP which is G at a position corresponding to position 155,860,812 bp of chromosome 8 of the B73 reference genome AGPv4;
MA0043, which is a SNP which is A at a position corresponding to position 155,999,733 bp of chromosome 8 of the B73 reference genome AGPv4;
MA0025, which is a SNP which is G at a position corresponding to position 156,967,599 bp of chromosome 8 of the B73 reference genome AGPv4;
PZE-108096469, which is a SNP which is G at a position corresponding to position 157,266,475 bp of chromosome 8 of the B73 reference genome AGPv4;
PZE-108096610, which is a SNP which is G at a position corresponding to position 157,375,673 bp of chromosome 8 of the B73 reference genome AGPv4; or
PZA-003182005, which is a SNP which is A at a position corresponding to position 158,167,578 bp of chromosome 8 of the B73 reference genome AGPv4.

As used herein, “maize” refers to a plant of the species Zea mays, preferably Zea mays ssp mays.

The term “plant” includes whole plants, including descendants or progeny thereof. As used herein unless clearly indicated otherwise, the term “plant” intends to mean a plant at any developmental stage. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc. “Parts of a plant” are e.g. shoot vegetative organs/structures, e.g., leaves, stems and tubers; roots, flowers and floral organs/structures, e.g. bracts, sepals, petals, stamens, carpels, anthers and ovules; seed, including embryo, endosperm, and seed coat; fruit and the mature ovary; plant tissue, e.g. vascular tissue, ground tissue, and the like; and cells, e.g. guard cells, egg cells, pollen, trichomes and the like; and progeny of the same. Parts of plants may be attached to or separate from a whole intact plant. Such parts of a plant include, but are not limited to, organs, tissues, and cells of a plant, and preferably seeds. A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. “Plant cell culture” means cultures of plant units such as, for example, protoplasts, cell culture cells, cells in plant tissues, pollen, pollen tubes, ovules, embryo sacs, zygotes and embryos at various stages of development. “Plant material” refers to leaves, stems, roots, flowers or flower parts, fruits, pollen, egg cells, zygotes, seeds, cuttings, cell or tissue cultures, or any other part or product of a plant. This also includes callus or callus tissue as well as extracts (such as extracts from taproots) or samples. A “plant organ” is a distinct and visibly structured and differentiated part of a plant such as a root, stem, leaf, flower bud, or embryo. “Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In certain embodiments, the plant part or derivative is or comprises (functional) propagation material, such as germplasm, a seed, or plant embryo or other material from which a plant can be regenerated. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc.

As used herein, the term plant population may be used interchangeably with population of plants. A plant population preferably comprises a multitude of individual plants, such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

As used herein, “HT2” refers to “Helminthosporium turcicum resistance 2”, which is a locus on Zea mays chromosome 8 responsible for Helminthosporium turcicum resistance. The causative gene responsible for conferring Helminthosporium turcicum resistance, which is located within the HT2 locus is RLK1 (receptor tyrosine kinase 1), or WAK-RLK1 (wall-associated kinase RLK1). As used herein, “HT3” refers to “Helminthosporium turcicum resistance 3”, which is a locus on Zea mays chromosome 8 responsible for Helminthosporium turcicum resistance. The causative gene responsible for conferring Helminthosporium turcicum resistance, which is located within the HT3 locus is RLK1 (receptor tyrosine kinase 1), or WAK-RLK1 (wall-associated kinase RLK1). The RLK1 Helminthosporium turcicum resistance conferring allele may comprise a gene sequence as set forth in SEQ ID NO: 1 or may comprise a coding sequence as set forth in SEQ ID NO: 2 or may encode a protein comprising a sequence as set forth in SEQ ID NO: 3. The coding sequence of RLK1 is identical in the HT2 and HT3 alleles. However, the HT2 and HT3 alleles derive from different genotypes/donor lines and differ at several locations outside the RLK1 coding sequence. In certain embodiments, the RLK1 gene, protein, or coding sequence has a sequence corresponding to the sequence of the RLK1 gene, protein, or coding sequence of the HT2 or HT3 allele.

The term “locus” (loci plural) means a specific place or places or a site on a chromosome where for example a QTL/haplotype, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question.

As used herein, the term “allele” or “alleles” refers to one or more alternative forms, i.e. different nucleotide sequences, of a locus.

An “allele of a locus” can comprise multiple genes or other genetic factors within a contiguous genomic region or linkage group, such as a haplotype. An allele of a locus can denote a haplotype within a specified window wherein said window is a contiguous genomic region that can be defined, and tracked, with a set of one or more polymorphic markers. A haplotype can be defined by the unique fingerprint of alleles at each marker within the specified window. A locus may encode for one or more alleles that affect the expressivity of a continuously distributed (quantitative) phenotype. In certain embodiments, the locus, allele, polynucleic acid, or molecular marker (allele) as described herein may be homozygous. In certain embodiments, the locus, allele, polynucleic acid, or molecular marker (allele) as described herein may be heterozygous.

As used herein, the term “mutant alleles” or “mutation” of alleles include alleles having one or more mutations, such as insertions, deletions, stop codons, base changes (e.g., transitions or transversions), or alterations in splice junctions, which may or may not give rise to altered gene products. Modifications in alleles may arise in coding or non-coding regions (e.g. promoter regions, exons, introns or splice junctions).

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant.

“Fine-mapping” refers to methods by which the position of a genomic region (e.g. QTL) can be determined more accurately (narrowed down) and by which the size of the introgression fragment comprising the QTL is reduced. For example Near Isogenic Lines for the QTL or haplotype (QTL/haplotype-NILs) can be made, which contain different, overlapping fragments of the introgression fragment within an otherwise uniform genetic background of the recurrent parent. Such lines can then be used to map on which fragment the QTL/haplotype is located and to identify a line having a shorter introgression fragment comprising the QTL/haplotype.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a molecular marker genetically linked to a genomic region (e.g. haplotype) or gene (e.g. the RLK1 allele conferring pathogen resistance) as defined herein, can be used to detect and/or select plants comprising the HT2/HT3 on chromosome 8. The closer the genetic linkage of the molecular marker to the locus (e.g. about 7 cM, 6 cM, 5 cM, 4 cM, 3 cM, 2 cM, 1 cM, 0.5 cM or less), the less likely it is that the marker is dissociated from the locus through meiotic recombination. Likewise, the closer two markers are linked to each other (e.g. within 7 or cM, 4 cM, 3 cM, 2 cM, 1 cM or less) the less likely it is that the two markers will be separated from one another (and the more likely they will co-segregate as a unit). A marker “within 7 cM or within 5 cM, 3 cM, 2 cM, or 1 cM” of another marker refers to a marker which genetically maps to within the 7 cM or 5 cM, 3 cM, 2 cM, or 1 cM region flanking the marker (i.e. either side of the marker). Similarly, a marker within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favour the presence of linkage and a LOD score greater than 3.0 is considered evidence for linkage. A LOD score of +3 indicates 1000 to 1 odds that the linkage being observed did not occur by chance.

A “marker haplotype” refers to a combination of (marker) alleles at a (marker) locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosomal segment are also “genetically linked”, typically within a genetic recombination distance of less than or equal to 50 cM, e.g., about 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25 cM or less. That is, two genetic elements within a single chromosomal segment undergo recombination during meiosis with each other at a frequency of less than or equal to about 50%, e.g., about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25% or less. “Closely linked” markers display a cross over frequency with a given marker of about 10% or less, e.g., 9%, 8%, 7%, 6%, 5%, 4%, 3%, 2%, 1%, 0.75%, 0.5%, 0.25% or less (the given marker locus is within about 10 cM of a closely linked marker locus, e.g., 9, 8, 7, 6, 5, 4, 3, 2, 1, 0.75, 0.5, 0.25 cM or less of a closely linked marker locus). Put another way, closely linked marker loci co-segregate at least about 90% the time, e.g., 91%, 92%, 93%, 94%, 95%, 96%, 97%, 98%, 99%, 99.5%, 99.75%, or more of the time.

The term “pathogen” as used herein generally refers to any type of infectious agent capable of causing an (infectious) disease, and includes without limitation a virus, bacterium, protozoan, prion, viroid, or fungus (including yeasts). Also parasites, such as insects or worms, but also parasitic plants or algae are generally encompassed by the term pathogen as used herein.

In certain embodiments, the pathogen as referred to herein is a fungal pathogen. In certain embodiments, the fungal pathogen belongs to the division of Ascomycota or Basidiomycota. The fungal pathogen may belong to family Pleosporaceae, Pucciniaceae or Botryosphaeriaceae. Preferably, the fungal pathogen belongs to the genus of Setosphaeria, Bipolaris, Puccinia or Diplodia, more preferably is the species of Helminthosporium turcicum, Setosphaeria rostrata, Setosphaeria glycinea, Setosphaeria holmii, Setosphaeria khartoumensis, Setosphaeria minor, Setosphaeria monoceras, Setosphaeria pedicellata, Setosphaeria prolata, Bipolaris australis, Bipolaris brizae, Bipolaris buchloes, Bipolaris cactivora, Bipolaris clavata, Bipolaris colds, Bipolaris colocasiae, Bipolaris crotonis, Bipolaris crustacean, Bipolaris cylindrical, Bipolaris euchlaenae, Bipolaris halepensis, Bipolaris heveae, Bipolaris incurvata, Bipolaris indica, Bipolaris iridis, Bipolaris leersiae, Bipolaris micropus, Bipolaris miyakei, Bipolaris multiformis, Bipolaris nicotiae, Bipolaris novae-zelandiae, Bipolaris ovariicola, Bipolaris panici-miliacei, Bipolaris papendorfii, Bipolaris sacchari, Bipolaris salkadehensis, Bipolaris sorghicola, Bipolaris subpapendorfii, Bipolaris tropicalis, Bipolaris urochloae, Bipolaris zeae, Puccinia asparagi, Puccinia graminis, Puccinia horiana, Puccinia mariae-wilsoniae, Puccinia poarum, Puccinia psidii, Puccinia recondite, Puccinia sessilis, Puccinia sorghi, Puccinia striiformis, Puccinia triticina, Diplodia maydis, Diplodia seriata or Stenocarpella (Diplodia) macrospora, most preferably is Helminthosporium turcicum, Puccinia sorghi, Diplodia macrospora or Bipolaris maydis.

In certain embodiments, the fungal pathogen causes a fungal plant disease. Accordingly, in certain embodiments, the plant disease as referred to herein is a fungal disease. In a preferred embodiment, the plant disease is selected from the group consisting of Northern Corn Leaf Blight (caused by Helminthosporium turcicum), Southern Corn Leaf Blight (caused by Bipolaris maydis), Common Rust (caused by Puccinia sorghi), and Diplodia Leaf Streak (caused by Diplodia macrospora, also called Stenocarpella macrospora). Most preferably, the plant disease is Northern Corn Leaf Blight (NCLB).

In certain embodiments, the term pathogen as used herein refers to a fungal pathogen. In certain embodiments, the term pathogen as used herein refers to a fungal pathogen of the genus Exserohilum (synonymous with Helminthosporium). In certain embodiments, the term pathogen as used herein refers to a fungal pathogen of the species Exserohilum turcicum (synonymous with Helminthosporium turcicum). In certain embodiments, the term pathogen as used herein refers to a pathogen causing Northern Corn Leaf Blight.

As used herein, Exserohilum turcicum can be used interchangeably with Helminthosporium turcicum. Exserohilum turcicum is the anamorph of Setosphaeria turcica. Exserohilum turcicum/Setosphaeria turcica belong to the phylum Ascomycota, the order of Pleosporales, and the family of Pleosporaceae. Exserohilum turcicum causes the plant disease Northern corn leaf blight. The most common diagnostic symptom of Northern corn leaf blight on maize is cigar-shaped (elliptical) necrotic gray-green lesions on the leaves of several cm long.

In an aspect, the invention relates to a method for generating a maize plant or plant part, preferably a maize plant having (increased) pathogen resistance and/or tolerance, preferably wherein said pathogen is Exserohilum turcicum, comprising introducing into the genome of a plant or a plant part a polynucleic acid according to the invention as described herein.

In certain embodiments, the polynucleic acid has a sequence comprised in a Zea mays chromosomal interval on chromosome 8 and flanked by molecular marker (allele) PZE-108092843 and PZA-003182005, such as a polynucleic acid comprising one or more of (HT2 or HT3) molecular marker (allele) MA0045, MA0062, MA0063, MA0070, MA0071, MA0064, PZE-108095339, and Affx-91328160, as also described herein elsewhere. The skilled person will understand that the polynucleic acid at least comprises the HT2/HT3 RLK1 gene, optionally comprising flanking sequences. In certain embodiments, the polynucleic acid does not comprise one or more of (HT2 or HT3) molecular marker (allele) PZE-108092843, PZA-003182005, PZE-108094590, PZE-108096610, PZE-108096469, MA0043 and MA0025, as also described herein elsewhere.

In certain embodiments, the polynucleic acid is introduced by introgression.

In certain embodiments, the invention relates to a method for generating a maize plant or plant part, preferably a maize plant or plant part having increased pathogen resistance and/or tolerance, preferably wherein said pathogen is Exserohilum turcicum, comprising (a) providing a first maize plant identified according to the method for identifying a maize plant or plant part as described herein elsewhere, (b) crossing said first maize plant with a second maize plant, (c) selecting progeny plants comprising a polynucleic acid according to the invention as described herein elsewhere, any one or more of molecular marker (allele) MA0045, MA0062, MA0063, MA0070, MA0071, MA0064, PZE-108095339, and Affx-91328160 or combinations as described herein elsewhere; and optionally (d) harvesting said maize plant part from said progeny.

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, a haplotype, a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A genetic element, an introgression fragment, or a gene or allele conferring a trait (such as increased pathogen resistance or tolerance) is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only pants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

In certain embodiments, the polynucleic acid is introduced (and genomically integrated) recombinantly or transgenically. The polynucleic acid may be introduced (and genomically integrated) at the native locus, to replace an endogenous polynucleic acid (such as the polynucleic acid not conferring pathogen resistance), or may be introduced (and genomically integrated) at a locus different than the endogenous locus (e.g. by random integration in the genome). In certain embodiments, the method for generating a maize plant or plant part comprises transforming a plant or plant part, preferably a plant cell, more preferably a protoplast, with the polynucleic acid, which may be provided on a vector, as described herein elsewhere. In certain embodiments, the polynucleic acid has a different sequence than an endogenous polynucleic acid (such as an endogenous polynucleic acid not conferring pathogen resisttance).

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). The term “transgenic” here means genetically modified by the introduction of a non-endogenous nucleic acid sequence. Typically a species-specific nucleic acid sequence is introduced in a form, arrangement or quantity into the cell in a location where the nucleic acid sequence does not occur naturally in the cell. This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically introduce one or more of the molecular marker (allele) according to the invention as described herein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues, i.e. a process in which a single-stranded nucleic acid molecule attaches itself to a complementary nucleic acid strand, i.e. agrees with this base pairing. Standard procedures for hybridization are described, for example, in Sambrook et al. (Molecular Cloning. A Laboratory Manual, Cold Spring Harbor Laboratory Press, 3rd edition 2001). The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence. Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. The possibility of such binding depends on the stringency of the hybridization conditions.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the marker (allele) of the invention as described herein may be provided on/as a template. By designing the system such that double strand breaks are introduced upstream and downstream of the corresponding region in the genome of a plant not comprising the marker (allele), this region is excised and can be replaced with the template comprising the marker (allele) of the invention. In this way, introduction of the marker (allele) of the invention in a plant need not involve multiple backcrossing, in particular in a plant of specific genetic background. Similarly, the polynucleic acid of the invention may be provided on/as a template. More advantageously however, the polynucleic acid of the invention may be generated without the use of a recombination template, but solely through the endonuclease action leading to a double strand DNA break which is repaired by NHEJ, resulting in the generation of indels.

In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18(4):455-7 and McCallum et al. “Targeting induced local lesions IN genomes (TILLING) for plant functional genomics”; Plant Physiol. 2000 June; 123(2):439-42.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the haplotype and/or one or more marker(s) as described herein is/are homozygous. In certain embodiments, the haplotype and/or one or more marker(s) as described herein are heterozygous. In certain embodiments, the haplotype allele and/or one or more marker(s) allele(s) as described herein is/are homozygous. In certain embodiments, the haplotype allele and/or one or more marker(s) allele(s) as described herein are heterozygous.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the Bl2seq program can be found in the readme file accompanying BLASTZ. Bl2seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq I .txt); -j is set to a file containing the second nucleic acid sequence to be compared (e.g., C:\seq2.txt); -p is set to blastn; -o is set to any desired file name (e.g., C:\output.txt); -q is set to - 1; -r is set to 2; and all other options are left at their default setting. The following command will generate an output file containing a comparison between two sequences: C:\B12seq c:\seqI .txt -j c:\seq2.txt -p blastn -o c:\output.txt -q - 1 -r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the Bl2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used. The terms “nucleotide sequence(s)”, “polynucleotide(s)”, “nucleic acid sequence(s)”, “nucleic acid(s)”, “nucleic acid molecule”, “polynucleic acid(s)” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%> nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides.

The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative<1>, in which an amino acid residue contained in the wild-type protein is replaced with another naturally-occurring amino acid of similar character, for example Gly<->Ala, Val<->Ile<->Leu, Asp<->Glu, Lys<->Arg, Asn<->Gln or Phe<->Trp<->Tyr. Substitutions encompassed by the present invention may also be “non-conservative”, in which an amino acid residue which is present in the wild-type protein is substituted with an amino acid with different properties, such as a naturally-occurring amino acid from a different group (e.g. substituting a charged or hydrophobic amino acid with alanine. “Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

A used herein, the term “endogenous” refers to a gene or allele which is present in its natural genomic location. The term “endogenous” can be used interchangeably with “native”. This does not however exclude the presence of one or more nucleic acid differences with the wild-type allele. In particular embodiments, the difference with a wild-type allele can be limited to less than 9 preferably less than 6, more particularly less than 3 nucleotide differences, such as 0 nucleotides difference. More particularly, the difference with the wildtype sequence can be in only one nucleotide. Preferably, the endogenous allele encodes a modified protein having less than 9, preferably less than 6, more particularly less than 3 and even more preferably only one or no amino acid difference with the wild-type protein.

A used herein, the term “exogenous polynucleotide” refers to a polynucleotide, such as a gene (or cDNA) or allele which is or has been recombinantly introduced in a cell (or plant). The exogenous polynucleotide may be episomal or genomically integrated. Integration may be random or site-directed. Integration may include replacement of a corresponding endogenous polynucleotide. It will be understood that an exogenous polynucleotide is not naturally present in the cell or plant.

As used herein, the B73 reference genome AGPv2 refers to the assembly B73 RefGen_v2 (also known as AGPv2, B73 RefGen_v2) as provided on the Maize Genetics and Genomics Database (https://www.maizegdb.org/genome/genome_assembly/B73%20RefGen_v2).

As used herein, the B73 reference genome AGPv4 (or AGPv04) refers to the assembly B73 RefGen_v2 (also known as AGPv4, B73 RefGen_v4) as provided on the Maize Genetics and Genomics Database (https://www.maizegdb.org/genome/genome_assembly/Zm-B73-REFERENCE-GRAM ENE-4.0).

Methods for screening for the presence of the polynucleic acid of the invention, or the (molecular) marker(s) (alleles) as described herein are known in the art. Without limitation, screening may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc.

As used herein the terms “increased pathogen tolerance” and “increased pathogen resistance” relate to any relief from, reduced presentation of, improvement of, or any combination thereof of any symptom (such as damage or loss in biomass) of an infection by a pathogen. Increased pathogen resistance or tolerance as referred to herein may also relate to the ability to which a plant maintains for instance its biomass production (such as harvestable biomass production, such as seed yield) upon or during pathogen infection. A pathogen resistant or tolerant plant, plant cell or plant part may refer herein to a plant, plant cell or plant part, respectively, having increased resistance/tolerance to a pathogen compared to a parent plant from which they are derived (e.g. not comprising the haplotype or one or more of the molecular marker (allele) or polynucleic acid according to the invention as described herein). Resistance may relate herein to the plant's ability to limit pathogen multiplication. Tolerance may relate herein to a plant's ability to reduce the effect of infection on its fitness regardless of the level of pathogen multiplication. Methods of determining pathogen resistance/tolerance are known to the person of skill in the art, such as visual scoring of pathogen infection or pathogen-induced damage, determination of biomass (yield), etc. As used herein, the terms “increased pathogen tolerance” and “increased pathogen resistance” may be used interchangeably with “reduced sensitivity” or “reduced susceptibility” towards pathogens. Accordingly, a plant, plant part, or plant population according to the invention which is more resistant or more tolerant towards a pathogen is considered less sensitive toward such pathogen. Less sensitive or less susceptible when used herein may be seen as “more tolerant” or “more resistant”. Similarly, “more tolerant” or “more resistant” may, vice versa, be seen as “less sensitive” or “less susceptible”. More sensitive or more susceptible when used herein may, vice versa, be seen as “less tolerant” or “less resistant”. Similarly, “less tolerant” or “less resistant” may, vice versa, be seen as “more sensitive” or “more susceptible”.

By means of example, and without limitation, an increase of pathogen resistance or tolerance can be evaluated based several criteria, such as (pathogenic) symptoms, which can for instance be classified according to the Table below.

Classification score scheme for phenotyping experiments in field trials at various locations with natural and artificial H. turcicum inoculation (from the Deutsche Maiskomitee (DMK, German maize committee); AG variety 27.02.02; (DMK J. Rath; R P Freiburg H. J. Imgraben)

| Classification | |
|---|---|
| score | Phenotype |
| 1 | Plant exhibits no symptoms of disease, 0% |
| 2 | Beginning of infestation, first small |
| | spots (less than 2 cm) visible. |
| | Less than 5% of leaf surface affected |
| 3 | Some spots have developed on a leaf stage. |
| | Between 5-10% of leaf surface affected. |
| 4 | 10-20% of leaf surface affected. Clearly |
| | visible spots on several leaf stages. |
| 5 | 20-40% of leaf surface affected. |
| | Spots start to coalesce. |
| 6 | 40-60% of leaf surface affected. |
| | Systemic infestation visible on |
| | leaves. |
| 7 | 60-80% of leaf surface affected. |
| | Approximately half of leaves |
| | destroyed or dried out because |
| | of fungal infestation. |
| 8 | 80-90% of leaf surface affected. |
| | More than half of leaves |
| | destroyed or dried out because |
| | of fungal infestation. |
| 9 | 90-100% of leaf surface affected. |
| | The plants are almost |
| | completely dried out. |

In certain embodiments, an increased pathogen resistance or tolerance entails a lower classification score in comparison with a plant lacking the increased resistance or tolerance, such as at least one, at least two, at least three, at least four, at least five or more classes, classifications points or scores.

As referred to herein, a polynucleic acid of the invention as described herein, is said to be flanked by certain molecular markers or molecular marker alleles if the polynucleic acid is comprised within a polynucleic acid wherein respectively a first marker (allele) is located upstream (i.e. 5′) of said polynucleic acid and a second marker (allele) is located downstream (i.e. 3′) of said polynucleic acid. Such first and second marker (allele) may border the polynucleic acid. The nucleic acid may equally comprise such first and second marker (allele), such as respectively at or near the 5′ and 3′ end, for instance respectively within 50 kb of the 5′ and 3′ end, preferably within 10 kb of the 5′ and 3′ end, such as within 5 kb of the 5′ and 3′ end, within 1 kb of the 5′ and 3′ end, or less.

In certain embodiments, introducing (into the genome) as referred to herein comprises transgenesis.

In certain embodiments, introducing (into the genome) as referred to herein comprises transformation.

In certain embodiments, introducing (into the genome) as referred to herein comprises recombination, such as homologous recombination.

In certain embodiments, introducing (into the genome) as referred to herein comprises mutagenesis.

In certain embodiments, introducing (into the genome) as referred to herein comprises introgression. In certain embodiments, introducing into the genome as referred to herein does not comprise introgression.

In certain embodiments, introducing into the genome as referred to herein comprises introducing into the genome in a plant part. In certain embodiments, the plant part is a plant organ. In certain embodiments, the plant part is a plant tissue. In certain embodiments, the plant part is a plant cell. In certain embodiments, the plant part is a protoplast.

In certain embodiments, introducing into the genome as referred to herein comprises introducing into the genome in vitro. In certain embodiments, introducing into the genome as referred to herein comprises introducing into the genome in vivo.

In certain embodiments, the method for generating a maize plant or plant part, such as a maize plant or plant part having (increased) pathogen resistance and/or tolerance, comprises transforming a plant or plant part, preferably a plant cell, more preferably a protoplast, with a polynucleic acid as described herein elsewhere, and optionally regenerating a plant from said plant cell, preferably protoplast.

In certain embodiments, the transformed plant or plant part does not endogenously comprise the polynucleic acid according to the invention as described herein.

In certain embodiments, the transformed plant or plant part does not endogenously comprise the one or more molecular marker (alleles) according to the invention as described herein.

In certain embodiments, the methods for obtaining or generating plants or plant parts as described herein according to the invention involve or comprise transgenesis and/or gene editing, such as including CRISPR/Cas, TALEN, ZFN, meganucleases; (induced) mutagenesis, which may or may not be random mutagenesis, such as TILLING.

In certain embodiments, the methods for obtaining plants or plant parts as described herein according to the invention do not involve or comprise transgenesis, gene editing, and/or mutagenesis.

In certain embodiments, the methods for obtaining plants or plant parts as described herein according to the invention involve, comprise or consist of breeding and/or selection.

In certain embodiments, the methods for obtaining plants or plant parts as described herein according to the invention do not involve, comprise or consist of breeding.

In an aspect, the invention relates to a maize plant or plant part obtained or obtainable by the methods according to the invention as described herein, such as the methods for identifying a maize plant or plant part or the methods for generating a maize plant or plant part. The invention also relates to the progeny of such plants.

In an aspect, the invention relates to a maize plant or plant part comprising a polynucleic acid according to the invention as described herein. In certain embodiments, the polynucleic acid allele is homozygous. In certain embodiments, the polynucleic acid allele is heterozygous.

In an aspect, the invention relates to a maize plant or plant part comprising any one or more molecular marker (allele) according to the invention as described herein. In certain embodiments, the molecular marker (allele) is homozygous. In certain embodiments, the molecular marker (allele) allele is heterozygous.

In certain embodiments, the maize plant or plant part is not naturally occurring. In certain embodiments, the maize plant or plant part is not a maize plant or plant part disclosed in WO 2019/038326.

In certain embodiments, the plant or plant part comprises one or more molecular marker (allele) upstream (i.e. 5′) of the RLK1 gene and one or more molecular marker (allele) downstream (i.e. 3′) of the RLK1 gene.

In certain embodiments, the maize plant or plant part does not comprise one or more of (resistance conferring or HT2 or HT3) molecular marker (allele) PZE-108092843, PZA-003182005, PZE-108094590, PZE-108096610, PZE-108096469, MA0043 and MA0025, as described herein elsewhere. In certain embodiments, the plant or plant part does not comprises one or more of these molecular marker (allele) upstream (i.e. 5′) of the RLK1 gene and one or more of these molecular marker (allele) downstream (i.e. 3′) of the RLK1 gene.

In certain embodiments, the maize plant is not a maize variety. In certain embodiments, the plant is not exclusively obtained by means of an essentially biological process. In certain embodiments, the plant is obtained by a method which contains at least one step other than crossing, i.e. the screening for the presence of a polynucleotide as described herein.

In certain embodiments, the maize plant or plant part is transgenic, gene-edited, or mutagenized. In certain embodiments, the maize plant or plant part is transgenic, gene-edited, or mutagenized in order to comprise the one or more molecular marker (allele), or one or more of the polynucleic acids according to the invention as described herein.

As described herein elsewhere, in certain embodiments such maize plant or plant part does not comprise endogenously the recited polynucleic acids.

In an aspect, the invention relates to a (isolated) polynucleic acid comprising one or more of the molecular marker (allele) according to the invention as described herein elsewhere.

In an aspect, the invention relates to a (isolated) polynucleic acid comprising one or more molecular marker (allele) according to the invention as described herein, or the complement or reverse complement thereof.

In certain embodiments, the invention relates to a (isolated) polynucleic acid comprising or specifically hybridizing with molecular marker (allele) MA0045, the complement, or reverse complement thereof. In certain embodiments, the invention relates to a (isolated) polynucleic acid comprising or specifically hybridizing with molecular marker (allele) MA0062, the complement, or reverse complement thereof. In certain embodiments, the invention relates to a (isolated) polynucleic acid comprising or specifically hybridizing with molecular marker (allele) MA0063, the complement, or reverse complement thereof. In certain embodiments, the invention relates to a (isolated) polynucleic acid comprising or specifically hybridizing with molecular marker (allele) MA0070, the complement, or reverse complement thereof. In certain embodiments, the invention relates to a (isolated) polynucleic acid comprising or specifically hybridizing with molecular marker (allele) MA0071, the complement, or reverse complement thereof. In certain embodiments, the invention relates to a (isolated) polynucleic acid comprising or specifically hybridizing with molecular marker (allele) MA0064, the complement, or reverse complement thereof. In certain embodiments, the invention relates to a (isolated) polynucleic acid comprising or specifically hybridizing with molecular marker (allele) PZE-108095339, the complement, or reverse complement thereof. In certain embodiments, the invention relates to a (isolated) polynucleic acid comprising or specifically hybridizing with molecular marker (allele) Affx-91328160, the complement, or reverse complement thereof.

In certain embodiments, the polynucleic acid comprises (or specifically hybridizes with a polynucleic acid having) one or more of the positions corresponding to the positions in the B73 maize reference genome (chromosome 8) AGPv04 as indicated in the Table below.

| marker name | position AGPv04 | HT2/HT3 allele | Alternative allele |
|---|---|---|---|
| Affx-91328160 | 156372823 | G | A |
| PZE-108095339 | 156378424 | G | A |
| MA0064 | 156541890 | C | T |
| MA0063 | 156543061 | T | C |
| MA0070 | 156694565 | T | G |
| MA0071 | 156694641 | C | T |
| MA0062 | 156772431 | G | T |
| MA0045 | 156789751 | A | C |

The nucleotides (SNPs) at the positions indicated for the HT2/HT3 allele allow screening for or the identification of the resistance phenotype according to the invention. The nucleotides (SNPs) at the positions indicated for the alternative allele allow screening for or the identification of the non-resistance phenotype. It will be understood that for identification of the non-resistance allele the indicated SNP nucleotides may be different than those indicated in the Table (as long as these are different than the SNP nucleotides indicated for the HT2/HT3 allele).

In certain embodiments, the invention relates to a (isolated) polynucleic acid comprising or specifically hybridizing with molecular marker (allele) MA0045 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0062 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0063 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0070 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0071 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0064 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) PZE-108095339 and at least 14 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) Affx-91328160 and at least 14 nucleotides upstream and/or downstream (referenced to the corresponding nucleotides of the B73 maize genome chromosome 8 AGPv04), the complement, or reverse complement thereof.

In certain embodiments, the invention relates to a (isolated) polynucleic acid comprising or specifically hybridizing with molecular marker (allele) MA0045 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0062 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0063 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0070 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0071 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) MA0064 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) PZE-108095339 and at least 15, preferably at least 17 nucleotides upstream and/or downstream (referenced to the corresponding nucleotides of the B73 maize genome chromosome 8 AGPv04), the complement, or reverse complement thereof. In certain embodiments, the invention relates to a (isolated) polynucleic acid comprising or specifically hybridizing with molecular marker (allele) Affx-91328160 and at least 15, preferably at least 17 nucleotides upstream and/or downstream (referenced to the corresponding nucleotides of the B73 maize genome chromosome 8 AGPv04), the complement, or reverse complement thereof.

In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) MA0045, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) MA0062, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) MA0063, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) MA0070, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) MA0071, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) MA0064, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) Affx-91328160, or the complement or reverse complement of the fragment. In certain embodiments, the invention relates to a (isolated) polynucleic acid comprising or specifically hybridizing with a fragment of genomic interval of Zea mays chromosome 8 between molecular markers PZE-108092843 and PZA-003182005 and comprising molecular marker (allele) PZE-108095339, or the complement or reverse complement of the fragment.

It will be understood that the polynucleic acids according to the invention comprises or specifically hybridizes with one or more of the (HT2/HT3 or alternative) molecular marker (allele) and additional 5′ and/or 3′ contiguous nucleotides (naturally) flanking the respective marker (allele) (or the complement or reverse complement thereof). In this context, the amount of flanking may in certain embodiments be at least 14 or 15 nucleotides (which may or may not be entirely 5′ or entirely 3′ flanking nucleotides, such as for instance 5 3′ flanking nucleotides plus 10 5′ flanking nucleotides. In certain embodiments, the molecular marker (allele) of the present invention (or the complement thereof) is the most 5′ nucleotide of the polynucleic acid. In certain embodiments, the molecular marker (allele) of the present invention (or the complement thereof) is the second most 5′ nucleotide of the polynucleic acid. In certain embodiments, the molecular marker (allele) of the present invention (or the complement thereof) is the third most 5′ nucleotide of the polynucleic acid. In certain embodiments, the molecular marker (allele) of the present invention (or the complement thereof) is the most 3′ nucleotide of the polynucleic acid. In certain embodiments, the molecular marker (allele) of the present invention (or the complement thereof) is the second most 3′ nucleotide of the polynucleic acid. In certain embodiments, the molecular marker (allele) of the present invention (or the complement thereof) is the third most 3′ nucleotide of the polynucleic acid.

In certain embodiments, the polynucleic acid comprises at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides.

In certain embodiments, the polynucleic acid comprises at most 1500 nucleotides, such as 1200, 1000, 800, 600, 400, 200 nucleotides, such as at most 100, 80, 60, 50, 40, or 30 nucleotides.

In certain embodiments, the polynucleic acid comprises at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides, and the polynucleic acid comprises at most 1500 nucleotides, such as 1200, 1000, 800, 600, 400, 200 nucleotides, such as at most 100, 80, 60, 50, 40, or 30 nucleotides.

In certain embodiments, the (isolated) polynucleotide has a length ranging from 15 to 500 nucleotides, preferably 15 to 100 nucleotides, preferably 15 to 50 nucleotides, more preferably 15 to 35 nucleotides.

In certain embodiments, the (isolated) polynucleotide is a primer or a probe.

In certain embodiments, the (isolated) polynucleotide is an allele-specific primer or probe.

In certain embodiments, the (isolated) is a KASP (Kompetitive allele specific PCR) primer. (KASP) primers are well-known in the art and can be designed by the skilled person according to known criteria. By means of further guidance, and without limitation, KASP is performed with two (or more) allele-specific primers (which may be the forward primers) and generally one common primer (which may be the reverse primer). The allele-specific primers are typically elongated with tail sequences (in which a different tail sequence is provided for each allele-specific primer). The tail sequences allow incorporation of a fluorescently labelled complementary sequence, to thereby fluorescently distinguish the different alleles.

In certain embodiments, the length of the tail sequence is comprised in the total primer length. In certain embodiments, the length of the tail sequence is not comprised in the total primer length.

In certain embodiments, the polynucleic acid is a (PCR) primer or (hybridization) probe. In certain embodiments, the polynucleic acid is an allele-specific primer or probe. In certain embodiments, the polynucleic acid is a KASP primer.

In an aspect, the invention relates to a (isolated) polynucleic acid comprising a (molecular) marker (allele) of the invention, or the complement or the reverse complement of a (molecular) marker (allele) of the invention. In certain embodiments, the invention relates to a polynucleic acid comprising at least 10 contiguous nucleotides, preferably at least 15 contiguous nucleotides or at least 20 contiguous nucleotides of a (molecular) marker (allele) of the invention, or the complement or the reverse complement of a (molecular) marker (allele) of the invention. In certain embodiments, the polynucleic acid is capable of discriminating between a (molecular) marker (allele) of the invention and a non-molecular marker allele, such as to specifically hybridise with a (molecular) marker allele of the invention. In certain embodiments, the polynucleic acid or the complement or reverse complement thereof does not (substantially) hybridise with or bind to (genomic) DNA originating from maize inbred line B73. In certain embodiments, the sequence of the polynucleic acid or the complement or reverse complement thereof does not occur or is not present in maize inbred line B73.

In certain embodiments, the polynucleic acid comprises a sequence as set forth in any of SEQ ID NOs: 4-51, the complement or reverse complement thereof. In certain embodiments, the polynucleic acid consists essentially of a sequence as set forth in any of SEQ ID NOs: 4-51, the complement or reverse complement thereof. In certain embodiments, the polynucleic acid consists of a sequence as set forth in any of SEQ ID NOs: 4-51, the complement or reverse complement thereof. In certain embodiments, the polynucleic acid comprises a fragment having at least the 15 most 3′ contiguous nucleotides of a sequence as set forth in any of SEQ ID NOs: 4-51, the complement or reverse complement thereof. In certain embodiments, the polynucleic acid consists essentially of a fragment having at least the 15 most 3′ contiguous nucleotides of a sequence as set forth in any of SEQ ID NOs: 4-51, the complement or reverse complement thereof. In certain embodiments, the polynucleic acid consists of a fragment having at least the 15 most 3′ contiguous nucleotides of a sequence as set forth in any of SEQ ID NOs: 4-51, the complement or reverse complement thereof.

In certain embodiments, the polynucleic acid comprises a sequence as set forth in any of SEQ ID NOs: 4, 5, 7, 8, 10, 11, 13, 14, 16, 17, 19, 20, 40, 41, 43, or 44, the complement or reverse complement thereof. In certain embodiments, the polynucleic acid consists essentially of a sequence as set forth in any of SEQ ID NOs: 4, 5, 7, 8, 10, 11, 13, 14, 16, 17, 19, 20, 40, 41, 43, or 44, the complement or reverse complement thereof. In certain embodiments, the polynucleic acid consists of a sequence as set forth in any of SEQ ID NOs: 4, 5, 7, 8, 10, 11, 13, 14, 16, 17, 19, 20, 40, 41, 43, or 44, the complement or reverse complement thereof. In certain embodiments, the polynucleic acid comprises a fragment having at least the 15 most 3′ contiguous nucleotides of a sequence as set forth in any of SEQ ID NOs: 4, 5, 7, 8, 10, 11, 13, 14, 16, 17, 19, 20, 40, 41, 43, or 44, the complement or reverse complement thereof. In certain embodiments, the polynucleic acid consists essentially of a fragment having at least the 15 most 3′ contiguous nucleotides of a sequence as set forth in any of SEQ ID NOs: 4, 5, 7, 8, 10, 11, 13, 14, 16, 17, 19, 20, 40, 41, 43, or 44, the complement or reverse complement thereof. In certain embodiments, the polynucleic acid consists of a fragment having at least the 15 most 3′ contiguous nucleotides of a sequence as set forth in any of SEQ ID NOs: 4, 5, 7, 8, 10, 11, 13, 14, 16, 17, 19, 20, 40, 41, 43, or 44, the complement or reverse complement thereof.

In certain embodiments, the polynucleic acid comprises a sequence as set forth in any of SEQ ID NOs: 22, 23, 25, 26, 28, 29, 31, 32, 34, 35, 37, 38, 46, 47, 49, or 50, the complement or reverse complement thereof. In certain embodiments, the polynucleic acid consists essentially of a sequence as set forth in any of SEQ ID NOs: 22, 23, 25, 26, 28, 29, 31, 32, 34, 35, 37, 38, 46, 47, 49, or 50, the complement or reverse complement thereof. In certain embodiments, the polynucleic acid consists of a sequence as set forth in any of SEQ ID NOs: 22, 23, 25, 26, 28, 29, 31, 32, 34, 35, 37, 38, 46, 47, 49, or 50, the complement or reverse complement thereof. In certain embodiments, the polynucleic acid comprises a fragment having at least the 15 most 3′ contiguous nucleotides of a sequence as set forth in any of SEQ ID NOs: 22, 23, 25, 26, 28, 29, 31, 32, 34, 35, 37, 38, 46, 47, 49, or 50, the complement or reverse complement thereof. In certain embodiments, the polynucleic acid consists essentially of a fragment having at least the 15 most 3′ contiguous nucleotides of a sequence as set forth in any of SEQ ID NOs: 22, 23, 25, 26, 28, 29, 31, 32, 34, 35, 37, 38, 46, 47, 49, or 50, the complement or reverse complement thereof. In certain embodiments, the polynucleic acid consists of a fragment having at least the 15 most 3′ contiguous nucleotides of a sequence as set forth in any of SEQ ID NOs: 22, 23, 25, 26, 28, 29, 31, 32, 34, 35, 37, 38, 46, 47, 49, or 50, the complement or reverse complement thereof.

In an aspect, the invention relates to a polynucleotide set, such as a primer set, comprising a combination of two or more of polynucleotides as described herein, such as polynucleotides comprising, consisting essentially of, or consisting of a sequence as set forth in any of SEQ ID NOs: 4-51, or fragments thereof comprising at least the 15, preferably at least 18 most 3′ contiguous nucleotides thereof, the complement or reverse complement thereof.

In certain embodiments, the polynucleotide set comprises a polynucleotide comprising, consisting essentially of, or consisting of a sequence as set forth in SEQ ID NO: 4 and a polynucleotide comprising, consisting essentially of, or consisting of a sequence as set forth in SEQ ID NO: 5, optionally further comprising a polynucleotide comprising, consisting essentially of, or consisting of a sequence as set forth in SEQ ID NO: 6; or (a) fragment(s) thereof comprising at least the 15 most 3′ contiguous nucleotides thereof, the complement or reverse complement thereof. [embodiment A]

In certain embodiments, the polynucleotide set comprises a polynucleotide comprising, consisting essentially of, or consisting of a sequence as set forth in SEQ ID NO: 22 and a polynucleotide comprising, consisting essentially of, or consisting of a sequence as set forth in SEQ ID NO: 23, optionally further comprising a polynucleotide comprising, consisting essentially of, or consisting of a sequence as set forth in SEQ ID NO: 24; or (a) fragment(s) thereof comprising at least the 15 most 3′ contiguous nucleotides thereof, the complement or reverse complement thereof. [embodiment B]

In certain embodiments, the polynucleotide set comprises a polynucleotide comprising, consisting essentially of, or consisting of a sequence as set forth in SEQ ID NO: 7 and a polynucleotide comprising, consisting essentially of, or consisting of a sequence as set forth in SEQ ID NO: 8, optionally further comprising a polynucleotide comprising, consisting essentially of, or consisting of a sequence as set forth in SEQ ID NO: 9; or (a) fragment(s) thereof comprising at least the 15 most 3′ contiguous nucleotides thereof, the complement or reverse complement thereof. [embodiment C]

In certain embodiments, the polynucleotide set comprises a polynucleotide comprising, consisting essentially of, or consisting of a sequence as set forth in SEQ ID NO: 25 and a polynucleotide comprising, consisting essentially of, or consisting of a sequence as set forth in SEQ ID NO: 26, optionally further comprising a polynucleotide comprising, consisting essentially of, or consisting of a sequence as set forth in SEQ ID NO: 27; or (a) fragment(s) thereof comprising at least the 15 most 3′ contiguous nucleotides thereof, the complement or reverse complement thereof. [embodiment D]

In certain embodiments, the polynucleotide set comprises a polynucleotide comprising, consisting essentially of, or consisting of a sequence as set forth in SEQ ID NO: 10 and a polynucleotide comprising, consisting essentially of, or consisting of a sequence as set forth in SEQ ID NO: 11, optionally further comprising a polynucleotide comprising, consisting essentially of, or consisting of a sequence as set forth in SEQ ID NO: 12; or (a) fragment(s) thereof comprising at least the 15 most 3′ contiguous nucleotides thereof, the complement or reverse complement thereof. [embodiment E]

In certain embodiments, the polynucleotide set comprises a polynucleotide comprising, consisting essentially of, or consisting of a sequence as set forth in SEQ ID NO: 28 and a polynucleotide comprising, consisting essentially of, or consisting of a sequence as set forth in SEQ ID NO: 29, optionally further comprising a polynucleotide comprising, consisting essentially of, or consisting of a sequence as set forth in SEQ ID NO: 30; or (a) fragment(s) thereof comprising at least the 15 most 3′ contiguous nucleotides thereof, the complement or reverse complement thereof. [embodiment F]

In certain embodiments, the polynucleotide set comprises a polynucleotide comprising, consisting essentially of, or consisting of a sequence as set forth in SEQ ID NO: 13 and a polynucleotide comprising, consisting essentially of, or consisting of a sequence as set forth in SEQ ID NO: 14, optionally further comprising a polynucleotide comprising, consisting essentially of, or consisting of a sequence as set forth in SEQ ID NO: 15; or (a) fragment(s) thereof comprising at least the 15 most 3′ contiguous nucleotides thereof, the complement or reverse complement thereof. [embodiment G]

In certain embodiments, the polynucleotide set comprises a polynucleotide comprising, consisting essentially of, or consisting of a sequence as set forth in SEQ ID NO: 31 and a polynucleotide comprising, consisting essentially of, or consisting of a sequence as set forth in SEQ ID NO: 32, optionally further comprising a polynucleotide comprising, consisting essentially of, or consisting of a sequence as set forth in SEQ ID NO: 33; or (a) fragment(s) thereof comprising at least the 15 most 3′ contiguous nucleotides thereof, the complement or reverse complement thereof. [embodiment H]

In certain embodiments, the polynucleotide set comprises a polynucleotide comprising, consisting essentially of, or consisting of a sequence as set forth in SEQ ID NO: 16 and a polynucleotide comprising, consisting essentially of, or consisting of a sequence as set forth in SEQ ID NO: 17, optionally further comprising a polynucleotide comprising, consisting essentially of, or consisting of a sequence as set forth in SEQ ID NO: 18; or (a) fragment(s) thereof comprising at least the 15 most 3′ contiguous nucleotides thereof, the complement or reverse complement thereof. [embodiment I]

In certain embodiments, the polynucleotide set comprises a polynucleotide comprising, consisting essentially of, or consisting of a sequence as set forth in SEQ ID NO: 34 and a polynucleotide comprising, consisting essentially of, or consisting of a sequence as set forth in SEQ ID NO: 35, optionally further comprising a polynucleotide comprising, consisting essentially of, or consisting of a sequence as set forth in SEQ ID NO: 36; or (a) fragment(s) thereof comprising at least the 15 most 3′ contiguous nucleotides thereof, the complement or reverse complement thereof. [embodiment J]

In certain embodiments, the polynucleotide set comprises a polynucleotide comprising, consisting essentially of, or consisting of a sequence as set forth in SEQ ID NO: 19 and a polynucleotide comprising, consisting essentially of, or consisting of a sequence as set forth in SEQ ID NO: 20, optionally further comprising a polynucleotide comprising, consisting essentially of, or consisting of a sequence as set forth in SEQ ID NO: 21; or (a) fragment(s) thereof comprising at least the 15 most 3′ contiguous nucleotides thereof, the complement or reverse complement thereof. [embodiment K]

In certain embodiments, the polynucleotide set comprises a polynucleotide comprising, consisting essentially of, or consisting of a sequence as set forth in SEQ ID NO: 37 and a polynucleotide comprising, consisting essentially of, or consisting of a sequence as set forth in SEQ ID NO: 38, optionally further comprising a polynucleotide comprising, consisting essentially of, or consisting of a sequence as set forth in SEQ ID NO: 39; or (a) fragment(s) thereof comprising at least the 15 most 3′ contiguous nucleotides thereof, the complement or reverse complement thereof. [embodiment L]

In certain embodiments, the polynucleotide set comprises a polynucleotide comprising, consisting essentially of, or consisting of a sequence as set forth in SEQ ID NO: 40 and a polynucleotide comprising, consisting essentially of, or consisting of a sequence as set forth in SEQ ID NO: 41, optionally further comprising a polynucleotide comprising, consisting essentially of, or consisting of a sequence as set forth in SEQ ID NO: 42; or (a) fragment(s) thereof comprising at least the 15 most 3′ contiguous nucleotides thereof, the complement or reverse complement thereof. [embodiment M]

In certain embodiments, the polynucleotide set comprises a polynucleotide comprising, consisting essentially of, or consisting of a sequence as set forth in SEQ ID NO: 43 and a polynucleotide comprising, consisting essentially of, or consisting of a sequence as set forth in SEQ ID NO: 44, optionally further comprising a polynucleotide comprising, consisting essentially of, or consisting of a sequence as set forth in SEQ ID NO: 45; or (a) fragment(s) thereof comprising at least the 15 most 3′ contiguous nucleotides thereof, the complement or reverse complement thereof. [embodiment N]

In certain embodiments, the polynucleotide set comprises a polynucleotide comprising, consisting essentially of, or consisting of a sequence as set forth in SEQ ID NO: 46 and a polynucleotide comprising, consisting essentially of, or consisting of a sequence as set forth in SEQ ID NO: 47, optionally further comprising a polynucleotide comprising, consisting essentially of, or consisting of a sequence as set forth in SEQ ID NO: 48; or (a) fragment(s) thereof comprising at least the 15 most 3′ contiguous nucleotides thereof, the complement or reverse complement thereof. [embodiment O]

In certain embodiments, the polynucleotide set comprises a polynucleotide comprising, consisting essentially of, or consisting of a sequence as set forth in SEQ ID NO: 49 and a polynucleotide comprising, consisting essentially of, or consisting of a sequence as set forth in SEQ ID NO: 50, optionally further comprising a polynucleotide comprising, consisting essentially of, or consisting of a sequence as set forth in SEQ ID NO: 51; or (a) fragment(s) thereof comprising at least the 15 most 3′ contiguous nucleotides thereof, the complement or reverse complement thereof. [embodiment P]

In certain embodiments, the polynucleotide set comprises a combination of any one or more of embodiments A to P above.

In certain embodiments, the polynucleotide set comprises one or a combination of the following embodiments: [A], [C], [E], [G], [I], [K], [M], [O], [A,C], [A,E], [A,G], [A,I], [A,K], [A,M], [A,O], [C,E], [C,G], [C,I], [C,K], [C,M], [C,O], [E,G], [E,I], [E,K], [E,M], [E,O], [G,I], [G,K], [G,M], [G,O], [I,K], [I,M], [I,O], [K,M], [K,O], [M,O], [A,C,E], [A,C,G], [A,C,I], [A,C,K], [A,C,M], [A,C,O], [A,E,G], [A,E,I], [A,E,K], [A,E,M], [A,E,O], [A,G,I], [A,G,K], [A,G,M], [A,G,O], [A,I,K], [A,I,M], [A,I,O], [A,K,M], [A,K,O], [A,M,O], [C,E,G], [C,E,I], [C,E,K], [C,E,M], [C,E,O], [C,G,I], [C,G,K], [C,G,M], [C,G,O], [C,I,K], [C,I,M], [C,I,O], [C,K,M], [C,K,O], [C,M,O], [E,G,I], [E,G,K], [E,G,M], [E,G,O], [E,I,K], [E,I,M], [E,I,O], [E,K,M], [E,K,O], [E,M,O], [G,I,K], [G,I,M], [G,I,O], [G,K,M], [G,K,O], [G,M,O], [I,K,M], [I,K,O], [I,M,O], [K,M,O], [A,C,E,G], [A,C,E,I], [A,C,E,K], [A,C,E, M], [A,C,E,O], [A,C,G,I], [A,C,G,K], [A,C,G,M], [A,C,G,O], [A,C,I,K], [A,C,I,M], [A,C,I,O], [A,C,K,M], [A,C,K,O], [A,C,M,O], [A,E,G,I], [A,E,G,K], [A,E,G,M], [A,E,G,O], [A,E,I,K], [A,E,I,M], [A,E,I,O], [A,E,K,M], [A,E,K,O], [A,E,M,O], [A,G,I,K], [A,G,I,M], [A,G,I,O], [A,G,K,M], [A,G,K,O], [A,G,M,O], [A,I,K,M], [A,I,K,O], [A,I,M,O], [A,K,M,O], [C,E,G,I], [C,E,G,K], [C,E,G,M], [C,E,G,O], [C,E,I,K], [C,E,I,M], [C,E,I,O], [C,E,K,M], [C,E,K,O], [C,E,M,O], [C,G,I,K], [C,G,I,M], [C,G,I,O], [C,G,K,M], [C,G,K,O], [C,G,M,O], [C,I,K,M], [C,I,K,O], [C,I,M,O], [C,K,M,O], [E,G,I,K], [E,G,I,M], [E,G,I,O], [E,G,K,M], [E,G,K,O], [E,G,M,O], [E,I,K,M], [E,I,K,O], [E,I,M,O], [E,K,M,O], [G,I,K,M], [G,I,K,O], [G,I,M,O], [G,K,M,O], [I,K,M,O], [A,C,E,G,I], [A,C,E,G,K], [A,C,E,G,M], [A,C,E,G,O], [A,C,E,I,K], [A,C,E,I,M], [A,C,E,I,O], [A,C,E,K,M], [A,C,E,K,O], [A,C,E,M,O], [A,C,G,I,K], [A,C,G,I,M], [A,C,G,I,O], [A,C,G,K,M], [A,C,G,K,O], [A,C,G,M,O], [A,C,I,K,M], [A,C,I,K,O], [A,C,I,M,O], [A,C,K,M,O], [A,E,G,I,K], [A,E,G,I,M], [A,E,G,I,O], [A,E,G,K,M], [A,E,G,K,O], [A,E,G,M,O], [A,E,I,K,M], [A,E,I,K,O], [A,E,I,M,O], [A,E,K,M,O], [A,G,I,K,M], [A,G,I,K,O], [A,G,I,M,O], [A,G,K,M,O], [A,I,K,M,O], [C,E,G,I,K], [C,E,G,I,M], [C,E,G,I,O], [C,E,G,K,M], [C,E,G,K,O], [C,E,G,M,O], [C,E,I,K,M], [C,E,I,K,O], [C,E,I,M,O], [C,E,K,M,O], [C,G,I,K,M], [C,G,I,K,O], [C,G,I,M,O], [C,G,K,M,O], [C,I,K,M,O], [E,G,I,K,M], [E,G,I,K,O], [E,G,I,M,O], [E,G,K,M,O], [E,I,K,M,O], [G,I,K,M,O], [A,C,E,G,I,K], [A,C,E,G,I,M], [A,C,E,G,I,O], [A,C,E,G,K,M], [A,C,E,G,K,O], [A,C,E,G,M,O], [A,C,E,I,K,M], [A,C,E,I,K,O], [A,C,E,I,M,O], [A,C,E,K,M,O], [A,C,G,I,K,M], [A,C,G,I,K,O], [A,C,G,I,M,O], [A,C,G,K,M,O], [A,C,I,K,M,O], [A,E,G,I,K,M], [A,E,G,I,K,O], [A,E,G,I,M,O], [A,E,G,K,M,O], [A,E,I,K,M,O], [A,G,I,K,M,O], [C,E,G,I,K,M], [C,E,G,I,K,O], [C,E,G,I,M,O], [C,E,G,K,M,O], [C,E,I,K,M,O], [C,G,I,K,M,O], [E,G,I,K,M,O], [A,C,E,G,I,K,M], [A,C,E,G,I,K,O], [A,C,E,G,I,M,O], [A,C,E,G,K,M,O], [A,C,E,I,K,M,O], [A,C,G,I,K,M,O], [A,E,G,I,K,M,O], [C,E,G,I,K,M,O], [A,C,E,G,I,K,M,O].

In certain embodiments, the polynucleotide set comprises one or a combination of the following embodiments: [B], [D], [F], [H], [J], [L], [N], [P], [B,D], [B,F], [B,H], [B,J], [B,L], [B,N], [B,P], [D,F], [D,H], [D,J], [D,L], [D,N], [D,P], [F,H], [F,J], [F,L], [F,N], [F,P], [H,J], [H,L], [H,N], [H,P], [J,L], [J,N], [J,P], [L,N], [L,P], [N,P], [B,D,F], [B,D,H], [B,D,J], [B,D,L], [B,D,N], [B,D,P], [B,F,H], [B,F,J], [B,F,L], [B,F,N], [B,F,P], [B,H,J], [B,H,L], [B,H,N], [B,H,P], [B,J,L], [B,J,N], [B,J,P], [B,L,N], [B,L,P], [B,N,P], [D,F,H], [D,F,J], [D,F,L], [D,F,N], [D,F,P], [D,H,J], [D,H,L], [D,H,N], [D,H,P], [D,J,L], [D,J,N], [D,J,P], [D,L,N], [D,L,P], [D,N,P], [F,H,J], [F,H,L], [F,H,N], [F,H,P], [F,J,L], [F,J,N], [F,J,P], [F,L,N], [F,L,P], [F,N,P], [H,J,L], [H,J,N], [H,J,P], [H,L,N], [H,L,P], [H,N,P], [J,L,N], [J,L,P], [J,N,P], [L,N,P], [B,D,F,H], [B,D,F,J], [B,D,F,L], [B,D,F,N], [B,D,F,P], [B,D,H,J], [B,D,H,L], [B,D,H,N], [B,D,H,P], [B,D,J,L], [B,D,J,N], [B,D,J,P], [B,D,L,N], [B,D,L,P], [B,D,N,P], [B,F,H,J], [B,F,H,L], [B,F,H,N], [B,F,H,P], [B,F,J,L], [B,F,J,N], [B,F,J,P], [B,F,L,N], [B,F,L,P], [B,F,N,P], [B,H,J,L], [B,H,J,N], [B,H,J,P], [B,H,L,N], [B,H,L,P], [B,H,N,P], [B,J,L,N], [B,J,L,P], [B,J,N,P], [B,L,N,P], [D,F,H,J], [D,F,H,L], [D,F,H,N], [D,F,H,P], [D,F,J,L], [D,F,J,N], [D,F,J,P], [D,F,L,N], [D,F,L,P], [D,F,N,P], [D,H,J,L], [D,H,J,N], [D,H,J,P], [D,H,L,N], [D,H,L,P], [D,H,N,P], [D,J,L,N], [D,J,L,P], [D,J,N,P], [D,L,N,P], [F,H,J,L], [F,H,J,N], [F,H,J,P], [F,H,L,N], [F,H,L,P], [F,H,N,P], [F,J,L,N], [F,J,L,P], [F,J,N,P], [F,L,N,P], [H,J,L,N], [H,J,L,P], [H,J,N,P], [H,L,N,P], [J,L,N,P], [B,D,F,H,J], [B,D,F,H,L], [B,D,F,H,N], [B,D,F,H,P], [B,D,F,J,L], [B,D,F,J,N], [B,D,F,J,P], [B,D,F,L,N], [B,D,F,L,P], [B,D,F,N,P], [B,D,H,J,L], [B,D,H,J,N], [B,D,H,J,P], [B,D,H,L,N], [B,D,H,L,P], [B,D,H,N,P], [B,D,J,L,N], [B,D,J,L,P], [B,D,J,N,P], [B,D,L,N,P], [B,F,H,J,L], [B,F,H,J,N], [B,F,H,J,P], [B,F,H,L,N], [B,F,H,L,P], [B,F,H,N,P], [B,F,J,L,N], [B,F,J,L,P], [B,F,J,N,P], [B,F,L,N,P], [B,H,J,L,N], [B,H,J,L,P], [B,H,J,N,P], [B,H,L,N,P], [B,J,L,N,P], [D,F,H,J,L], [D,F,H,J,N], [D,F,H,J,P], [D,F,H,L,N], [D,F,H,L,P], [D,F,H,N,P], [D,F,J,L,N], [D,F,J,L,P], [D,F,J,N,P], [D,F,L,N,P], [D,H,J,L,N], [D,H,J,L,P], [D,H,J,N,P], [D,H,L,N,P], [D,J,L,N,P], [F,H,J,L,N], [F,H,J,L,P], [F,H,J,N,P], [F,H,L,N,P], [F,J,L,N,P], [H,J,L,N,P], [B,D,F,H,J,L], [B,D,F,H,J,N], [B,D,F,H,J,P], [B,D,F,H,L,N], [B,D,F,H,L,P], [B,D,F,H,N,P], [B,D,F,J,L,N], [B,D,F,J,L,P], [B,D,F,J,N,P], [B,D,F,L,N,P], [B,D,H,J,L,N], [B,D,H,J,L,P], [B,D,H,J,N,P], [B,D,H,L,N,P], [B,D,J,L,N,P], [B,F,H,J,L,N], [B,F,H,J,L,P], [B,F,H,J,N,P], [B,F,H,L,N,P], [B,F,J,L,N,P], [B,H,J,L,N,P], [D,F,H,J,L,N], [D,F,H,J,L,P], [D,F,H,J,N,P], [D,F,H,L,N,P], [D,F,J,L,N,P], [D,H,J,L,N,P], [F,H,J,L,N,P], [B,D,F,H,J,L,N], [B,D,F,H,J,L,P], [B,D,F,H,J,N,P], [B,D,F,H,L,N,P], [B,D,F,J,L,N,P], [B,D,H,J,L,N,P], [B,F,H,J,L,N,P], [D,F,H,J,L,N,P], [B,D,F,H,J,L,N,P].

In an aspect, the invention relates to a polynucleotide, such as a polynucleotide according to the invention as defined herein elsewhere, in particular a polynucleotide comprising molecular marker (allele) MA0045, MA0062, MA0063, MA0070, MA0071, MA0063, PZE-108095339, or Affx-91328160, comprising, consisting essentially of, or consisting of a sequence of any of SEQ ID NOs: 52 to 59 or (unique) fragments thereof, preferably of at least 15, more preferably at least 18 contiguous nucleotides, the complement or reverse complement thereof or of the fragment.

In certain embodiments, the isolated polynucleotide comprises at least
R at position 21 of SEQ ID NO: 52, preferably as the most 3′ or 5′, 2nd most 3′ or 5′, or 3rd most 3′ or 5′ nucleotide;
R at position 21 of SEQ ID NO: 53, preferably as the most 3′ or 5′, 2nd most 3′ or 5′, or 3rd most 3′ or 5′ nucleotide;
Y at position 21 of SEQ ID NO: 54, preferably as the most 3′ or 5′, 2nd most 3′ or 5′, or 3rd most 3′ or 5′ nucleotide;
Y at position 21 of SEQ ID NO: 55, preferably as the most 3′ or 5′, 2nd most 3′ or 5′, or 3rd most 3′ or 5′ nucleotide;
K at position 21 of SEQ ID NO: 56, preferably as the most 3′ or 5′, 2nd most 3′ or 5′, or 3rd most 3′ or 5′ nucleotide;
M at position 21 of SEQ ID NO: 57, preferably as the most 3′ or 5′, 2nd most 3′ or 5′, or 3rd most 3′ or 5′ nucleotide;
K at position 31 of SEQ ID NO: 58, preferably as the most 3′ or 5′, 2nd most 3′ or 5′, or 3rd most 3′ or 5′ nucleotide; or
Y at position 31 of SEQ ID NO: 59, preferably as the most 3′ or 5′, 2nd most 3′ or 5′, or 3rd most 3′ or 5′ nucleotide.

In certain embodiments, the isolated polynucleotide comprises at least
G at position 21 of SEQ ID NO: 52, preferably as the most 3′ or 5′, 2nd most 3′ or 5′, or 3rd most 3′ or 5′ nucleotide;
G at position 21 of SEQ ID NO: 53, preferably as the most 3′ or 5′, 2nd most 3′ or 5′, or 3rd most 3′ or 5′ nucleotide;
C at position 21 of SEQ ID NO: 54, preferably as the most 3′ or 5′, 2nd most 3′ or 5′, or 3rd most 3′ or 5′ nucleotide;
C at position 21 of SEQ ID NO: 55, preferably as the most 3′ or 5′, 2nd most 3′ or 5′, or 3rd most 3′ or 5′ nucleotide;
G at position 21 of SEQ ID NO: 56, preferably as the most 3′ or 5′, 2nd most 3′ or 5′, or 3rd most 3′ or 5′ nucleotide;
A at position 21 of SEQ ID NO: 57, preferably as the most 3′ or 5′, 2nd most 3′ or 5′, or 3rd most 3′ or 5′ nucleotide;
G at position 31 of SEQ ID NO: 58, preferably as the most 3′ or 5′, 2nd most 3′ or 5′, or 3rd most 3′ or 5′ nucleotide; or
C at position 31 of SEQ ID NO: 59, preferably as the most 3′ or 5′, 2nd most 3′ or 5′, or 3rd most 3′ or 5′ nucleotide.

In certain embodiments, the isolated polynucleotide comprises at least
A at position 21 of SEQ ID NO: 52, preferably as the most 3′ or 5′, 2nd most 3′ or 5′, or 3rd most 3′ or 5′ nucleotide;
A at position 21 of SEQ ID NO: 53, preferably as the most 3′ or 5′, 2nd most 3′ or 5′, or 3rd most 3′ or 5′ nucleotide;
T at position 21 of SEQ ID NO: 54, preferably as the most 3′ or 5′, 2nd most 3′ or 5′, or 3rd most 3′ or 5′ nucleotide;
T at position 21 of SEQ ID NO: 55, preferably as the most 3′ or 5′, 2nd most 3′ or 5′, or 3rd most 3′ or 5′ nucleotide;
T at position 21 of SEQ ID NO: 56, preferably as the most 3′ or 5′, 2nd most 3′ or 5′, or 3rd most 3′ or 5′ nucleotide;
C at position 21 of SEQ ID NO: 57, preferably as the most 3′ or 5′, 2nd most 3′ or 5′, or 3rd most 3′ or 5′ nucleotide;
T at position 31 of SEQ ID NO: 58, preferably as the most 3′ or 5′, 2nd most 3′ or 5′, or 3rd most 3′ or 5′ nucleotide; or
T at position 31 of SEQ ID NO: 59, preferably as the most 3′ or 5′, 2nd most 3′ or 5′, or 3rd most 3′ or 5′ nucleotide.

Such polynucleotides can be used to detects the HT2/HT3 allele or alternatively a different, non-HT2/HT3 allele, such as a B73 allele.

In an aspect, the invention relates to a kit comprising one or more of the polynucleotides as described herein, such as one or more of the primers or probes as described herein. The skilled person will understand that the polynucleotides may be comprised for instance in a single receptacle, such as a single vial, or in separate receptacles, such as separate vials.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

In certain embodiments, the (isolated) polynucleic acid is an (isolated) polynucleic acid which is not disclosed in WO 2019/038326.

In an aspect, the invention relates to a set of primers or probes as described above, such as a set of allele-specific primers or probes. In certain embodiments, the set may further comprise a (common) forward or reverse primer (depending on whether the allele-specific primers are reverse or forward primers).

In an aspect, the invention relates to a kit comprising such polynucleic acids, such as primers (comprising forward (such as one or more allele-specific or alternatively common primers) and/or reverse primers (such as a common or alternatively one or more allele-specific primers)) and/or probes (such as one or more allele-specific probe). The kit may further comprise instructions for use.

In will be understood that in embodiments relating to a set of forward and reverse primers, only one of both primers (forward or reverse) may need to be capable of discriminating between a (molecular) marker allele of the invention and a non-marker allele, and hence may be unique. The other primer may or may not be capable of discriminating between a (molecular) marker allele of the invention and a non-marker allele, and hence may be unique.

In an aspect, the invention relates to a vector comprising a (isolated) polynucleic acid according to the invention as described herein. In certain embodiments, the vector is a (plant) expression vector. In certain embodiments, the vector is an inducible (plant) expression vector. In certain embodiments, the expression is tissue- or organ-specific. In certain embodiments, the expression is developmentally specific. In certain embodiments, the expression is tissue- or organ-specific and developmentally specific.

As used herein, a “vector” has its ordinary meaning in the art, and may for instance be a plasmid, a cosmid, a phage or an expression vector, a transformation vector, shuttle vector, or cloning vector; it may be double- or single-stranded, linear or circular; or it may transform a prokaryotic or eukaryotic host, either via integration into its genome or extrachromosomally. The nucleic acid according to the invention is preferably operatively linked in a vector with one or more regulatory sequences which allow the transcription, and, optionally, the expression, in a prokaryotic or eukaryotic host cell. A regulatory sequence—preferably DNA—may be homologous or heterologous to the nucleic acid according to the invention. For example, the nucleic acid is under the control of a suitable promoter or terminator. Suitable promoters may be promoters which are constitutively induced (example: 35S promoter from the “Cauliflower mosaic virus” (Odell et al., 1985); those promoters which are tissue-specific are especially suitable (example: Pollen-specific promoters, Chen et al. (2010), Zhao et al. (2006), or Twell et al. (1991)), or are development-specific (example: blossom-specific promoters). Suitable promoters may also be synthetic or chimeric promoters which do not occur in nature, are composed of multiple elements, and contain a minimal promoter, as well as—upstream of the minimum promoter—at least one cis-regulatory element which serves as a binding location for special transcription factors. Chimeric promoters may be designed according to the desired specifics and are induced or repressed via different factors. Examples of such promoters are found in Gurr & Rushton (2005) or Venter (2007). For example, a suitable terminator is the nos-terminator (Depicker et al., 1982). The vector may be introduced via conjugation, mobilization, biolistic transformation, agrobacteria-mediated transformation, transfection, transduction, vacuum infiltration, or electroporation. The vector may be a plasmid, a cosmid, a phage or an expression vector, a transformation vector, shuttle vector, or cloning vector; it may be double- or single-stranded, linear or circular. The vector may transform a prokaryotic or eukaryotic host, either via integration into its genome or extrachromosomally.

As used herein, the term “operatively linked” or “operably linked” means connected in a common nucleic acid molecule in such a manner that the connected elements are positioned and oriented relative to one another such that a transcription of the nucleic acid molecule may occur. A DNA which is operatively linked with a promoter is under the transcriptional control of this promoter.

In an aspect, the invention relates to the use of the polynucleic acid or vector according to the invention as described herein for generating a maize plant or plant part, such as a maize plant or plant part having increased pathogen resistance and/or tolerance.

In certain embodiments, the vector is an expression vector. The nucleic acid is preferably operatively linked in a vector with one or more regulatory sequences which allow the transcription, and optionally the expression, in a prokaryotic or eukaryotic host cell. A regulatory sequence may be homologous or heterologous to the nucleic acid. For example, the nucleic acid is under the control of a suitable promoter or terminator. Suitable promoters may be promoters which are constitutively induced, for example, the 35S promoter from the “Cauliflower mosaic virus” (Odell et al., 1985. Identification of DNA sequences required for activity of the cauliflower mosaic virus 35S promoter.) Tissue-specific promoters, e.g. pollen-specific promoters as described in Chen et al. (2010. Molecular Biology Reports 37(2):737-744), Zhao et al. (2006. Planta 224(2): 405-412), or Twell et al. (1991. Genes & Development 5(3): 496-507), are particularly suitable, as are development-specific promoters, e.g. blossom-specific promoters. Suitable promoters may also be synthetic or chimeric promoters which do not occur in nature, and which are composed of multiple elements. Such synthetic or chimeric promoter may contain a minimal promoter, as well as at least one cis-regulatory element which serves as a binding location for special transcription factors. Chimeric promoters may be designed according to the desired specifics and can be induced or repressed via different factors. Examples of such promoters are found in Gurr & Rushton (2005. Trends in Biotechnology 23(6): 275-282) or Venter (2007. Trends in Plant Science: 12(3): 118-124). For example, a suitable terminator is the nos-terminator (Depicker et al., 1982. Journal of Molecular and Applied Genetics 1(6): 561-573).

In certain embodiments, the vector is a conditional expression vector. In certain embodiments, the vector is a constitutive expression vector. In certain embodiments, the vector is a tissue-specific expression vector, such as a pollen-specific expression vector. In certain embodiments, the vector is an inducible expression vector. All such vectors are well-known in the art.

Methods for preparation of the described vectors are commonplace to the person skilled in the art (Sambrook et al., 2001).

Also envisaged herein is a host cell, such as a plant cell, which comprises a nucleic acid as described herein, preferably an induction-promoting nucleic acid or a nucleic acid encoding a double-stranded RNA as described herein, or a vector as described herein. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

The host cell may be a prokaryotic (for example, bacterial) or eukaryotic cell (for example, a plant cell or a yeast cell). For example, the host cell may be an agrobacterium, such as Agrobacterium tumefaciens or Agrobacterium rhizogenes. Preferably, the host cell is a plant cell.

In an aspect, the invention relates to the use of the polynucleic acid or vector according to the invention as described herein for identifying a maize plant or plant part, such as a maize plant or plant part having increased pathogen resistance and/or tolerance.

A nucleic acid described herein or a vector described herein may be introduced in a host cell via well-known methods, which may depend on the selected host cell, including, for example, conjugation, mobilization, biolistic transformation, agrobacteria-mediated transformation, transfection, transduction, vacuum infiltration, or electroporation. In particular, methods for introducing a nucleic acid or a vector in an agrobacterium cell are well-known to the skilled person and may include conjugation or electroporation methods. Also methods for introducing a nucleic acid or a vector into a plant cell are known (Sambrook et al., 2001) and may include diverse transformation methods such as biolistic transformation and agrobacterium-mediated transformation.

In particular embodiments, the present invention relates to a transgenic plant cell which comprises a nucleic acid as described herein, in particular an induction-promoting nucleic acid or a nucleic acid encoding a double-stranded RNA as described herein, as a transgene or a vector as described herein. In further embodiments, the present invention relates to a transgenic plant or a part thereof which comprises the transgenic plant cell.

For example, such a transgenic plant cell or transgenic plant is a plant cell or plant which is, preferably stably, transformed with a nucleic acid as described herein, in particular an induction-promoting nucleic acid or a nucleic acid encoding a double-stranded RNA as described herein, or a vector as described herein.

Preferably, the nucleic acid in the transgenic plant cell is operatively linked with one or more regulatory sequences which allow the transcription, and optionally the expression, in the plant cell. A regulatory sequence may be homologous or heterologous to the nucleic acid. The total structure made up of the nucleic acid according to the invention and the regulatory sequence(s) may then represent the transgene.

In an aspect, the invention relates to the use of one or more of the (molecular) marker (allele) described herein for identifying a plant or plant part having increased pathogen resistance and/or tolerance. In an aspect, the invention relates to the use of one or more of the (molecular) marker (allele) described herein which are able to detect at least one diagnostic marker allele for identifying a plant or plant part, such as having increased pathogen resistance and/or tolerance. In an aspect, the invention relates to the detection of one or more of the (molecular) marker alleles described herein for identifying a plant or plant part having increased pathogen resistance and/or tolerance.

The marker alleles of the invention as described herein may be diagnostic marker alleles which are useable for identifying plants or plant parts having increased pathogen resistance and/or tolerance.

In an aspect, the invention relates to a maize plant or plant part, such as a protoplast, comprising the polynucleic acid or vector according to the invention as described herein.

In an aspect, the invention relates to a method for controlling pathogen infestation in a maize plant (population) comprising

a) Providing (a) maize plant(s) according to the invention as described herein or growing from seeds (a) maize plant(s) according to the invention as described herein,
b) Cultivating the plant(s) of a) under conditions of pathogen infestation.

In certain embodiments, pathogen infestation is reduced. In certain embodiments, pathogen infestation is delayed. In certain embodiments, pathogen symptoms are reduced. In certain embodiments, pathogen symptoms are delayed. In certain embodiments, infestation and symptoms are reduced or delayed. In certain embodiments, the number of lesions is reduced, such as the average or mean number of lesions per plant or the average or mean number of lesions in a plant population (such as for instance expressed in number of plants or per growth area, such as per hectare). A reduction in number of lesions can be at least 5%, preferably at least 10%, more preferably at least 20%. A delay in pathogen infestation or symptoms can be at least one week, preferably at least two weeks, more preferably at least one month.

The plants, plant parts or plant populations as described herein having increased pathogen resistance or tolerance can be used to control pathogen infestation or infection. Accordingly, in an aspect, the invention relates to the use of such plants for controlling pathogen infestation or infection. Preferably pathogen infestation or infection is controlled by reduction of pathogen infestation or infection or as reduction in the symptoms of pathogen infestation or infection, at the plant, plant part, or plant population level, such as further described below.

In certain embodiments, an increased resistance or tolerance may present itself as a reduction of infection or infestation (e.g. the amount of pathogens (e.g. per plant area or per plant biomass), the multiplication (rate) or spread (rate)/distribution of pathogens, as well as the speed of pathogen spreading such as at a specific time during the (growth) season) at the (sub) plant level (such as for instance particular cells, organs, or tissues, for instance harvestable parts of a plant or for instance leaves, stalks, fruits, or seeds) or at the population level, such as a reduction of at least 5%, preferably at least 10%, such as at least 20%, at least 30%, at least 40%, at least 50%, at least 60%, at least 70%, at least 80%, at least 90%, or (about) 100%. At the population level, an increased resistance or tolerance may present itself as a reduction of infection or infestation as described above, but also for instance as a reduction in the amount of infected plants (or a combination), such as a reduction of at least 5%, preferably at least 10%, such as at least 20%, at least 30%, at least 40%, at least 50%, at least 60%, at least 70%, at least 80%, at least 90%, or (about) 100%. It will be understood that such reduction of infection or infestation can be relative to a reference plant (part) or population (such as a corresponding wild type plant not according to the invention).

In certain embodiments, an increased resistance or tolerance may present itself as a reduction in the loss of biomass or yield in general or of a particular (harvestable) plant part (such as seed or fruit amount or weight) due to or as a consequence of pathogen infection. In certain embodiments, the resistant or tolerant plants exhibit a loss in biomass production (such as expressed in g/day or kg/ha or kg/ha/day, such as expressed as dry matter for instance expressed as weight percent) under pathogen infection which is at least 1%, preferably at least 2%, such as at least 3%, at least 4%, at least 5%, such as at least 10%, at least 15%, or at least 20% or more, lower than corresponding control plants, such as plants which are less resistant or tolerant, or plants not according to the invention as described herein. In certain embodiments, the resistant or tolerant plants exhibit biomass production (such as expressed in g/day or kg/ha or kg/ha/day, such as expressed as dry matter for instance expressed as weight percent) under pathogen infection which is at least 1%, preferably at least 2%, such as at least 3%, at least 4%, at least 5%, such as at least 10%, at least 15%, or at least 20% or more, higher than corresponding control plants, such as plants which are less resistant or tolerant, or plants not according to the invention as described herein.

As used herein, the term “yield potential” refers to maximum yield obtainable at harvest.

In an aspect, the invention relates to the use of a maize plant according to the invention as described herein for controlling pathogen infestation in a maize plant (population).

In an aspect, the invention relates to the use of a maize plant according to the invention as described herein for increasing the yield (potential) of a plant, preferably under conditions of pathogen infestation. In certain embodiments, the yield is biomass or seed yield. In certain embodiments, said biomass is whole plant biomass or biomass of a plant part. In certain embodiments, said plant part is a tissue, organ, fruit, or seed.

In certain embodiments of the methods, (maize) plants (or plant parts), polynucleic acids, or uses as described herein, the plant or plant part or polynucleic acid or (screening) method comprises (screening for) molecular marker (allele) MA0045. In certain embodiments of the methods, (maize) plants (or plant parts), polynucleic acids, or uses as described herein, the plant or plant part or polynucleic acid or (screening) method comprises (screening for) molecular marker (allele) MA0062. In certain embodiments of the methods, (maize) plants (or plant parts), polynucleic acids, or uses as described herein, the plant or plant part or polynucleic acid or (screening) method comprises (screening for) molecular marker (allele) MA0063. In certain embodiments of the methods, (maize) plants (or plant parts), polynucleic acids, or uses as described herein, the plant or plant part or polynucleic acid or (screening) method comprises (screening for) molecular marker (allele) MA0070. In certain embodiments of the methods, (maize) plants (or plant parts), polynucleic acids, or uses as described herein, the plant or plant part or polynucleic acid or (screening) method comprises (screening for) molecular marker (allele) MA0071. In certain embodiments of the methods, (maize) plants (or plant parts), polynucleic acids, or uses as described herein, the plant or plant part or polynucleic acid or (screening) method comprises (screening for) molecular marker (allele) MA0064. In certain embodiments of the methods, (maize) plants (or plant parts), polynucleic acids, or uses as described herein, the plant or plant part or polynucleic acid or (screening) method comprises (screening for) molecular marker (allele) PZE-108095339. In certain embodiments of the methods, (maize) plants (or plant parts), polynucleic acids, or uses as described herein, the plant or plant part or polynucleic acid or (screening) method comprises (screening for) molecular marker (allele) Affx-91328160.

In certain embodiments of the methods, (maize) plants (or plant parts), polynucleic acids, or uses as described herein, the plant or plant part or polynucleic acid or (screening) method comprises (screening for) molecular marker (allele) MA0045 and MA0062. In certain embodiments of the methods, (maize) plants (or plant parts), polynucleic acids, or uses as described herein, the plant or plant part or polynucleic acid or (screening) method comprises (screening for) molecular marker (allele) MA0045 and MA0063. In certain embodiments of the methods, (maize) plants (or plant parts), polynucleic acids, or uses as described herein, the plant or plant part or polynucleic acid or (screening) method comprises (screening for) molecular marker (allele) MA0045 and MA0064. In certain embodiments of the methods, (maize) plants (or plant parts), polynucleic acids, or uses as described herein, the plant or plant part or polynucleic acid or (screening) method comprises (screening for) molecular marker (allele) MA0045 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062 and MA0063. In certain embodiments of the methods, (maize) plants (or plant parts), polynucleic acids, or uses as described herein, the plant or plant part or polynucleic acid or (screening) method comprises (screening for) molecular marker (allele) MA0062 and MA0064. In certain embodiments of the methods, (maize) plants (or plant parts), polynucleic acids, or uses as described herein, the plant or plant part or polynucleic acid or (screening) method comprises (screening for) molecular marker (allele) MA0062 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0062 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0063 and MA0064. In certain embodiments of the methods, (maize) plants (or plant parts), polynucleic acids, or uses as described herein, the plant or plant part or polynucleic acid or (screening) method comprises (screening for) molecular marker (allele) MA0063 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0063 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0064 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0064 and Affx-91328160. In certain embodiments of the methods, (maize) plants (or plant parts), polynucleic acids, or uses as described herein, the plant or plant part or polynucleic acid or (screening) method comprises (screening for) molecular marker (allele) PZE-108095339 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070 and MA0045. In certain embodiments of the methods, (maize) plants (or plant parts), polynucleic acids, or uses as described herein, the plant or plant part or polynucleic acid or (screening) method comprises (screening for) molecular marker (allele) MA0070 and MA0062. In certain embodiments of the methods, (maize) plants (or plant parts), polynucleic acids, or uses as described herein, the plant or plant part or polynucleic acid or (screening) method comprises (screening for) molecular marker (allele) MA0070 and MA0063. In certain embodiments of the methods, (maize) plants (or plant parts), polynucleic acids, or uses as described herein, the plant or plant part or polynucleic acid or (screening) method comprises (screening for) molecular marker (allele) MA0070 and MA0064. In certain embodiments of the methods, (maize) plants (or plant parts), polynucleic acids, or uses as described herein, the plant or plant part or polynucleic acid or (screening) method comprises (screening for) molecular marker (allele) MA0070 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071 and MA0045. In certain embodiments of the methods, (maize) plants (or plant parts), polynucleic acids, or uses as described herein, the plant or plant part or polynucleic acid or (screening) method comprises (screening for) molecular marker (allele) MA0071 and MA0062. In certain embodiments of the methods, (maize) plants (or plant parts), polynucleic acids, or uses as described herein, the plant or plant part or polynucleic acid or (screening) method comprises (screening for) molecular marker (allele) MA0071 and MA0063. In certain embodiments of the methods, (maize) plants (or plant parts), polynucleic acids, or uses as described herein, the plant or plant part or polynucleic acid or (screening) method comprises (screening for) molecular marker (allele) MA0071 and MA0064. In certain embodiments of the methods, (maize) plants (or plant parts), polynucleic acids, or uses as described herein, the plant or plant part or polynucleic acid or (screening) method comprises (screening for) molecular marker (allele) MA0071 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071 and MA0070.

In certain embodiments of the methods, (maize) plants (or plant parts), polynucleic acids, or uses as described herein, the plant or plant part or polynucleic acid or (screening) method comprises (screening for) molecular marker (allele) MA0045, MA0062, and MA0063. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, and MA0064. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0064, PZE-108095339, and Affx-91328160. In certain embodiments of the methods, (maize) plants (or plant parts), polynucleic acids, or uses as described herein, the plant or plant part or polynucleic acid comprises molecular marker (allele) MA0070, MA0045, and MA0062. In certain embodiments of the methods, (maize) plants (or plant parts), polynucleic acids, or uses as described herein, the plant or plant part or polynucleic acid comprises molecular marker (allele) MA0070, MA0045, and MA0063. In certain embodiments of the methods, (maize) plants (or plant parts), polynucleic acids, or uses as described herein, the plant or plant part or polynucleic acid comprises molecular marker (allele) MA0070, MA0045, and MA0064. In certain embodiments of the methods, (maize) plants (or plant parts), polynucleic acids, or uses as described herein, the plant or plant part or polynucleic acid comprises molecular marker (allele) MA0070, MA0045, and PZE-108095339. In certain embodiments of the methods, (maize) plants (or plant parts), polynucleic acids, or uses as described herein, the plant or plant part or polynucleic acid comprises molecular marker (allele) MA0070, MA0045, and Affx-91328160. In certain embodiments of the methods, (maize) plants (or plant parts), polynucleic acids, or uses as described herein, the plant or plant part or polynucleic acid comprises molecular marker (allele) MA0070, MA0062, and MA0063. In certain embodiments of the methods, (maize) plants (or plant parts), polynucleic acids, or uses as described herein, the plant or plant part or polynucleic acid comprises molecular marker (allele) MA0070, MA0062, and MA0064. In certain embodiments of the methods, (maize) plants (or plant parts), polynucleic acids, or uses as described herein, the plant or plant part or polynucleic acid comprises molecular marker (allele) MA0070, MA0062, and PZE-108095339. In certain embodiments of the methods, (maize) plants (or plant parts), polynucleic acids, or uses as described herein, the plant or plant part or polynucleic acid comprises molecular marker (allele) MA0070, MA0062, and Affx-91328160. In certain embodiments of the methods, (maize) plants (or plant parts), polynucleic acids, or uses as described herein, the plant or plant part or polynucleic acid comprises molecular marker (allele) MA0070, MA0063, and MA0064. In certain embodiments of the methods, (maize) plants (or plant parts), polynucleic acids, or uses as described herein, the plant or plant part or polynucleic acid comprises molecular marker (allele) MA0070, MA0063, and PZE-108095339. In certain embodiments of the methods, (maize) plants (or plant parts), polynucleic acids, or uses as described herein, the plant or plant part or polynucleic acid comprises molecular marker (allele) MA0070, MA0063, and Affx-91328160. In certain embodiments of the methods, (maize) plants (or plant parts), polynucleic acids, or uses as described herein, the plant or plant part or polynucleic acid comprises molecular marker (allele) MA0070, MA0064, and PZE-108095339. In certain embodiments of the methods, (maize) plants (or plant parts), polynucleic acids, or uses as described herein, the plant or plant part or polynucleic acid comprises molecular marker (allele) MA0070, MA0064, and Affx-91328160. In certain embodiments of the methods, (maize) plants (or plant parts), polynucleic acids, or uses as described herein, the plant or plant part or polynucleic acid comprises molecular marker (allele) MA0070, PZE-108095339, and Affx-91328160. In certain embodiments of the methods, (maize) plants (or plant parts), polynucleic acids, or uses as described herein, the plant or plant part or polynucleic acid comprises molecular marker (allele) MA0071, MA0045, and MA0062. In certain embodiments of the methods, (maize) plants (or plant parts), polynucleic acids, or uses as described herein, the plant or plant part or polynucleic acid comprises molecular marker (allele) MA0071, MA0045, and MA0063. In certain embodiments of the methods, (maize) plants (or plant parts), polynucleic acids, or uses as described herein, the plant or plant part or polynucleic acid comprises molecular marker (allele) MA0071, MA0045, and MA0064. In certain embodiments of the methods, (maize) plants (or plant parts), polynucleic acids, or uses as described herein, the plant or plant part or polynucleic acid comprises molecular marker (allele) MA0071, MA0045, and PZE-108095339. In certain embodiments of the methods, (maize) plants (or plant parts), polynucleic acids, or uses as described herein, the plant or plant part or polynucleic acid comprises molecular marker (allele) MA0071, MA0045, and Affx-91328160. In certain embodiments of the methods, (maize) plants (or plant parts), polynucleic acids, or uses as described herein, the plant or plant part or polynucleic acid comprises molecular marker (allele) MA0071, MA0062, and MA0063. In certain embodiments of the methods, (maize) plants (or plant parts), polynucleic acids, or uses as described herein, the plant or plant part or polynucleic acid comprises molecular marker (allele) MA0071, MA0062, and MA0064. In certain embodiments of the methods, (maize) plants (or plant parts), polynucleic acids, or uses as described herein, the plant or plant part or polynucleic acid comprises molecular marker (allele) MA0071, MA0062, and PZE-108095339. In certain embodiments of the methods, (maize) plants (or plant parts), polynucleic acids, or uses as described herein, the plant or plant part or polynucleic acid comprises molecular marker (allele) MA0071, MA0062, and Affx-91328160. In certain embodiments of the methods, (maize) plants (or plant parts), polynucleic acids, or uses as described herein, the plant or plant part or polynucleic acid comprises molecular marker (allele) MA0071, MA0063, and MA0064. In certain embodiments of the methods, (maize) plants (or plant parts), polynucleic acids, or uses as described herein, the plant or plant part or polynucleic acid comprises molecular marker (allele) MA0071, MA0063, and PZE-108095339. In certain embodiments of the methods, (maize) plants (or plant parts), polynucleic acids, or uses as described herein, the plant or plant part or polynucleic acid comprises molecular marker (allele) MA0071, MA0063, and Affx-91328160. In certain embodiments of the methods, (maize) plants (or plant parts), polynucleic acids, or uses as described herein, the plant or plant part or polynucleic acid comprises molecular marker (allele) MA0071, MA0064, and PZE-108095339. In certain embodiments of the methods, (maize) plants (or plant parts), polynucleic acids, or uses as described herein, the plant or plant part or polynucleic acid comprises molecular marker (allele) MA0071, MA0064, and Affx-91328160. In certain embodiments of the methods, (maize) plants (or plant parts), polynucleic acids, or uses as described herein, the plant or plant part or polynucleic acid comprises molecular marker (allele) MA0071, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, and MA0045. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, and MA0062. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, and MA0063. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, and Affx-91328160.

In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and MA0063. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and MA0063. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and MA0064. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0062. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0063. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and MA0063. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0064 and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0064 and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, PZE-108095339 and Affx-91328160.

In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0045, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, PZE-108095339, and Affx-91328160.

In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and MA0064. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, and PZE-108095339. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0062, MA0045, MA0063, MA0064, PZE-108095339, and Affx-91328160.

In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0045, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0045, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0064, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, PZE-108095339, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, and Affx-91328160. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, and PZE-108095339.

In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, PZE-108095339, and Affx-91328160.

In certain embodiments, the methods as described herein, such as the methods for identifying a maize plant or plant part, further comprise screening for the presence of any one or more marker (allele) selected from PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid comprises molecular marker (allele) MA0045 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) Affx-91328160 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid comprises molecular marker (allele) MA0045 and MA0062 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045 and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045 and MA0070 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045 and MA0071 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045 and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and MA0070 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and MA0071 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063 and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063 and MA0070 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063 and MA0071 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0064 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0064 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) PZE-108095339 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0064 and MA0070 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0064 and MA0071 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071 and MA0070 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid comprises molecular marker (allele) MA0045, MA0062, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0045, and MA0062 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0045, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0045, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0045, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0045, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0062, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0062, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0062, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0062, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0045, and MA0062 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0045, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0045, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0045, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0045, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0062, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0062, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0062, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0062, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0070, MA0071, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, PZE-108095339, and MA0070 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0070 and MA0045 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0070 and MA0062 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0070 and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0071, MA0070 and MA0064 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid comprises molecular marker (allele) MA0045, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0062 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and MA0063 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0064 and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0064 and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, PZE-108095339 and Affx-91328160 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid comprises molecular marker (allele) MA0045, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid comprises molecular marker (allele) MA0062, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and MA0064 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0062, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, and Affx-91328160 and does not comprise any one or more of (HT2 or HT3) marker (allele) PZE-108092843, PZA-003182005, PZE-108094590, PZE-108096610, PZE-108096469, MA0043 and MA0025. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, and PZE-108095339 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, PZE-108095339, and Affx-91328160 and does not comprise any one or more of (HT2 or HT3) marker (allele) PZE-108092843, PZA-003182005, PZE-108094590, PZE-108096610, PZE-108096469, MA0043 and MA0025.

In certain embodiments of the methods, (maize) plants (or plant parts), polynucleic acids, or uses as described herein, the plant or plant part or polynucleic acid comprises molecular marker (allele) MA0045 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071 and does not comprise (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid comprises molecular marker (allele) MA0045 and MA0062 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045 and MA0063 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045 and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045 and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045 and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and MA0063 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063 and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063 and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063 and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0064 and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0064 and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) PZE-108095339 and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063 and MA0070 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063 and MA0071 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and MA0070 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062 and MA0071 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045 and MA0070 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045 and MA0071 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0064 and MA0070 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0064 and MA0071 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070 and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071 and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070 and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071 and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071 and MA0070 and does not comprise any one or more of (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid comprises molecular marker (allele) MA0045, MA0062, and MA0063 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, and Affx-91328160 and does not comprise marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0045, and MA0062 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0045, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0045, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0045, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0045, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0062, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0062, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0062, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0062, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0070, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0045, and MA0062 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0045, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0045, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0045, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0045, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0062, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0062, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0062, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0062, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0070, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0070, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0070, and MA0045 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0070, and MA0062 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0070, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0071, MA0070, and MA0064 and does not comprise any one or more of (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid comprises molecular marker (allele) MA0045, MA0062, MA0063, and MA0064 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0063, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0063, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0063, MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0062 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045 and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and MA0063 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062 and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063 and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0064 and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0064 and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, PZE-108095339 and Affx-91328160 and does not comprise any one or more of (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid comprises molecular marker (allele) MA0045, MA0062, MA0063, MA0064, and PZE-108095339 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0063, MA0064, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0063, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0062, MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0045, MA0063, MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid comprises molecular marker (allele) MA0062, MA0063, MA0064, PZE-108095339, and Affx-91328160 and does not comprise (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0063, MA0064, PZE-108095339, and Affx-91328160 and does not comprise any one or more of (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0045, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0045, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and MA0064 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0063, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0062, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, and PZE-108095339 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0062, MA0045, MA0063, MA0064, PZE-108095339, and Affx-91328160 and does not comprise any one or more of (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid or (screening) method comprises (screening for) molecular marker (allele) MA0071, MA0062, MA0045, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0062, MA0045, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0045, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0063, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0064, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, PZE-108095339, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, and Affx-91328160 and does not comprise any one or more of (HT2 or HT3) marker (allele) PZE-108092843 and/or PZA-003182005. In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, and PZE-108095339 and does not comprise any one or more of (HT2 or HT3) marker (allele) PZE-108092843 and/or PZA-003182005.

In certain embodiments of the methods, (maize) plants (or plant parts), polynucleic acids, or uses as described herein, the plant or plant part or polynucleic acid or (screening) method comprises (screening for) molecular marker (allele) MA0070, MA0071, MA0062, MA0045, MA0063, MA0064, PZE-108095339, and Affx-91328160 and does not comprise any one or more of (HT2 or HT3) marker (allele) PZE-108092843 and/or PZA-003182005.

The aspects and embodiments of the invention are further supported by the following non-limiting examples. The following examples, including the experiments conducted and the results achieved, are provided for illustrative purposes only and are not constructed as limiting the present invention.

### EXAMPLES

### Example 1: Development of Diagnostic Markers for the RLK1 HT2 Allele for Marker Assisted Selection and Trait Introgression in Breeding

The map-based cloning of the RLK1 HT2 allele in the QTL interval between marker PZE-108092843 and PZA-003182005 was further narrowed down by recombinant plants between the marker MA0035 and MA0045 (population RP6 x RP6HT3A) and PZE-108095325 and MA0045 (population RP5 x RP5HT2A). Recombinant plants with these intervals showed the resistant phenotype for NCLB in the field.

Marker-assisted selection for small donor introgression comprising HT2 from donor A619HT2 and A619HT3 1 could be performed with marker combinations between Affx-91328160 and MA0045 (see Table 1) in order to eliminate linkage drag positioned outside of the respective donor introgression. Such linkage drag is described already in WO 2019038326 A1.

Maize plants comprising the donor introgression with HT2 are characterized as having the marker haplotype comprising MA0064, MA0063, MA0062 and MA0045, optionally comprising additionally Affx-91328160 and PZE-108095339, as indicated in Table 1, wherein the donor introgression is located between the markers PZE-108092843 and PZA-003182005 (length: ˜3.72 Mb, calculated on basis of B73 AGPv4), preferably between the markers PZE-108094590 and PZE-108096610 (length: ˜1.52 Mb) or between the markers PZE-108094590 and PZE-108096469 (length: ˜1.41 Mb), more preferably between the markers MA0043 and MA0025 (length: ˜0.97 Mb).

The position of the HT2 gene according to the annotation to reference genome B73 AGPv4 is from 156,763,311 to 156,774,071 bp.

TABLE 1
List of all marker and their physical position on B73 AGPv4:

| | | position | Donor (HT2) |
|---|---|---|---|
| marker name | chromosome | AGPv04 | allele |
| PZE-108092843 ⁽¹⁾ | 8 | 154456106 | C |
| PZE-108094590 ⁽¹⁾ | 8 | 155860812 | G |
| MA0043 | 8 | 155999733 | A |
| Affx-91328160 ⁽²⁾ | 8 | 156372823 | G |
| PZE-108095325 ⁽¹⁾ | 8 | 156374083 | G |
| PZE-108095339 ⁽¹⁾ | 8 | 156378424 | G |
| MA0021 | 8 | 156535845 | G |
| MA0064 | 8 | 156541890 | C |
| MA0035 | 8 | 156542253 | A |
| MA0063 | 8 | 156543061 | T |
| MA0070 | 8 | 156694565 | T |
| MA0071 | 8 | 156694641 | C |
| MA0062 | 8 | 156772431 | G |
| MA0045 | 8 | 156789751 | A |
| MA0025 | 8 | 156967599 | G |
| PZE-108096469 ⁽¹⁾ | 8 | 157266475 | G |
| PZE-108096610 ⁽¹⁾ | 8 | 157375673 | G |
| PZA-003182005 ⁽¹⁾ | 8 | 158167578 | A |

⁽¹⁾ Ganal et al. (2011) “A Large Maize (Zea mays L.) SNP Genotyping Array: Development and Germplasm Genotyping, and Genetic Mapping to Compare with the B73 Reference Genome”, PLoS ONE, 6(12): e28334.
⁽²⁾ Unterseer et al. (2014) “A Powerful Tool for Genome Analysis in Maize: Development and Evaluation of the High Density 600 K SNP Genotyping Array”, BMC Genomics, 15(1): 823.

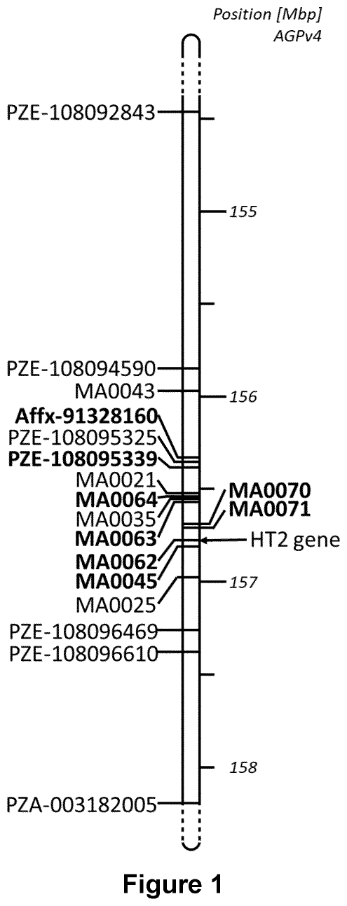

FIG. 1 shows a map of marker positions with reference to the marker positions of AGPv04.

Further marker development within the region (AGPv4 156372823-156789751 bp=Affx-91328160-MA0045) resulted in a diagnostic marker haplotype for the HT2 donor lines A619HT2 and A619HT3 of markers Affx-91328160, PZE-108095339, MA0064, MA0063, MA0062 and MA0045 (see Table 2).

TABLE 2
List of diagnostic markers contributing to the HT2 haplotype.

| | Donor | alternative |
|---|---|---|
| | allele | allele |
| | (i.e. | (i.e. non- |
| | HT2/HT3 | HT2/HT3 |
| Marker | allele) | allele) |
| Affx-91328160 | G | A |
| PZE-108095339 | G | A |
| MA0064 | C | T |
| MA0063 | T | C |
| MA0070 | T | G |
| MA0071 | C | T |
| MA0062 | G | T |
| MA0045 | A | C |

Each of these diagnostic markers can be used to follow a small donor introgression of at least HT2 (and HT3) in breeding processes. Since each marker is diagnostic and closely linked to the causative gene conferring HT2 resistance. Further, individual markers of Table 1 can be used in combination and/or in combination with other markers in the region from PZE-108092843 to PZA-003182005, preferably from PZE-108094590 to PZE-108096610 or PZE-108094590 to PZE-108096469, more preferably from MA0043 to MA0025.

KASP primers were developed for the detection of respectively the HT2 (and HT3) allele and the alternative allele, and are provided in Table 3. The common primer is also provided.

TABLE 3
List of KASP primers for differential detection of
diagnostic markers contributing to the HT2 (and HT3)
haplotype and alternatively the non-HT2 (and HT3)
haplotype.

| | HT2/HT3 allele | alternative | |
|---|---|---|---|
| Marker | primer | allele primer | Common primer |
| Affx- | GAAGGTCGGAGTCAA | GAAGGTGACCAAGTT | CTCCACAGTTTTTACTC |
| 91328160 | CGGATTGGGAAGATT | CATGCTAAGGGAAGA | CCAAGTTTAGAA (SEQ |
| | CATATTTGTTTACAT | TTCATATTTGTTTAC | ID NO: 6) |
| | CACG (SEQ ID | ATCACA (SEQ ID | |
| | NO: 4) | NO: 5) | |
| PZE- | GAAGGTCGGAGTCAA | GAAGGTGACCAAGTT | GTTTCGGGTTTCAGAAG |
| 108095339 | CGGATTCTTGGCGTG | CATGCTTGGCGTGCT | TCAAACGGTT (SEQ |
| | CTGCAGGACC (SEQ | GCAGGACT (SEQ | ID NO: 9) |
| | ID NO: 7) | ID NO: 8) | |
| MA0064 | GAAGGTCGGAGTCAA | GAAGGTGACCAAGTT | GCTAGAACATGGCGTAT |
| | CGGATTATGAACTTC | CATGCTGATGAACTT | ACGGAACAA (SEQ |
| | TTTTTGGTCGTTTTC | CTTTTTGGTCGTTTT | ID NO: 12) |
| | GC (SEQ ID NO: | CGT (SEQ ID NO: | |
| | 10) | 11) | |
| MA0063 | GAAGGTCGGAGTCAA | GAAGGTGACCAAGTT | GTGAACCCAGCCAACCT |
| | CGGATTGGTGAGCAG | CATGCTGTGAGCAGC | CGTCTT (SEQ ID |
| | CCATGGCGGA (SEQ | CATGGCGGG (SEQ | NO: 15) |
| | ID NO: 13) | ID NO: 14) | |
| MA0062 | GAAGGTGACCAAGTT | GAAGGTCGGAGTCAA | CACGTCAACCAGAAACA |
| | CATGCTATCTAATCA | CGGATTAAATCTAAT | TAAAGCACAAAT (SEQ |
| | AACTGGTGTTATATC | CAAACTGGTGTTATA | ID NO: 18) |
| | TTTTGG (SEQ ID | TCTTTTGT (SEQ | |
| | NO: 16) | ID NO: 17) | |
| MA0045 | GAAGGTGACCAAGTT | GAAGGTCGGAGTCAA | CCAAGAACTCTATTAGA |
| | CATGCTAACATCCCA | CGGATTACATCCCAA | TTGTTGAATAGCA |
| | AAGCACTGAACGGA | AGCACTGAACGGC | (SEQ ID NO: 21) |
| | (SEQ ID NO: 19) | (SEQ ID NO: 20) | |
| MA0070 | GAAGGTCGGAGTCAA | GAAGGTGACCAAGTT | CRAACCAAAGTTACCGC |
| | CGGATTCAATTTTCT | CATGCTCAATTTTCT | TATAAGCC (SEQ ID |
| | AAAAAGATTGGCAGC | AAAAAGATTGGCAGC | NO: 42) |
| | AATTAATT (SEQ | AATTAATG (SEQ | |
| | ID NO: 40) | ID NO: 41) | |
| MA0071 | GAAGGTGACCAAGTT | GAAGGTCGGAGTCAA | GCTTATAGCGGTAACTT |
| | CATGCTGAGCTAGCT | CGGATTGAGCTAGCT | TGGTTYGC (SEQ ID |
| | TGAGCTCGACCG | TGAGCTCGACCA | NO: 45) |
| | (SEQ ID NO: 43) | (SEQ ID NO: 44) | |

In Table 3, primer underlined nucleotide sections are hybridizing with Zea mays genomic DNA (either the direct or complementary strand). Nucleotide sections in bold represent KASP primer tail sequences (not binding to Zea mays genomic DNA). Nucleotides which are both underlined and in bold are the allele-specific nucleotides.

The primers listed in Table 3 are KASP primers. However, in certain embodiments, the primers are not KASP primers, and may lack the tail sequences. Accordingly, in certain embodiments, primers for differential detection may be as provided in Table 4.

TABLE 4
List of primers for differential detection of diagnostic
markers contributing to the HT2 (and HT3) haplotype and
alternatively the non-HT2 (and HT3) haplotype.

| | HT2/HT3 allele | alternative | |
|---|---|---|---|
| Marker | primer | allele primer | Common primer |
| Affx- | GGGAAGATTCATATT | AAGGGAAGATTCATAT | CTCCACAGTTTTTACT |
| 91328160 | TGTTTACATCACG | TTGTTTACATCACA | CCCAAGTTTAGAA |
| | (SEQ ID NO: 22) | (SEQ ID NO: 23) | (SEQ ID NO: 24) |
| PZE- | CTTGGCGTGCTGCAG | TGGCGTGCTGCAGGAC | GTTTCGGGTTTCAGAA |
| 108095339 | GACC (SEQ ID | T (SEQ ID NO: | GTCAAACGGTT (SEQ |
| | NO: 25) | 26) | ID NO: 27) |
| MA0064 | ATGAACTTCTTTTTG | GATGAACTTCTTTTTG | GCTAGAACATGGCGTA |
| | GTCGTTTTCGC | GTCGTTTTCGT (SEQ | TACGGAACAA (SEQ |
| | (SEQ ID NO: 28) | ID NO: 29) | ID NO: 30) |
| MA0063 | GGTGAGCAGCCATGG | GTGAGCAGCCATGGCG | GTGAACCCAGCCAACC |
| | CGGA (SEQ ID | GG (SEQ ID NO: | TCGTCTT (SEQ ID |
| | NO: 31) | 32) | NO: 33) |
| MA0062 | ATCTAATCAAACTGG | AAATCTAATCAAACTG | CACGTCAACCAGAAAC |
| | TGTTATATCTTTTGG | GTGTTATATCTTTTGT | ATAAAGCACAAAT |
| | (SEQ ID NO: 34) | (SEQ ID NO: 35) | (SEQ ID NO: 36) |
| MA0045 | AACATCCCAAAGCAC | ACATCCCAAAGCACTG | CCAAGAACTCTATTAG |
| | TGAACGGA (SEQ | AACGGC (SEQ ID | ATTGTTGAATAGCA |
| | ID NO: 37) | NO: 38) | (SEQ ID NO: 39) |
| MA0070 | CAATTTTCTAAAAAG | CAATTTTCTAAAAAGA | CRAACCAAAGTTACCG |
| | ATTGGCAGCAATTAA | TTGGCAGCAATTAATG | CTATAAGCC (SEQ |
| | TT (SEQ ID NO: | (SEQ ID NO: 47) | ID NO: 48) |
| | 46) | | |
| MA0071 | GAGCTAGCTTGAGCT | GAGCTAGCTTGAGCTC | GCTTATAGCGGTAACT |
| | CGACCG (SEQ ID | GACCA (SEQ ID | TTGGTTYGC (SEQ |
| | NO: 49) | NO: 50) | ID NO: 51) |

TABLE 5
List of surrounding sequences suitable
for differential detection of diagnostic
markers contributing to the HT2 (and HT3)
haplotype and alternatively the non-HT2
(and HT3) haplotype. The respective marker
positions (SNPs) are indicated in bold
underlined. Degenerate codes are indicated
according to IUPAC (R = A/G, Y = C/T,
K = G/T, M = A/C).

| Marker | Marker region |
|---|---|
| Affx- | TTCATATTTGTTTACATCACRATTCTAAACTTGGGAGTA |
| 91328160 | AA (SEQ ID NO: 52) |
| PZE- | ACGTGGCGCCAGCCACGCACRGTCCTGCAGCACGCCAAG |
| 108095339 | CA (SEQ ID NO: 53) |
| MA0064 | TTCTTTTTTGGTCGTTTTCGYTCCTTTGTTCCGTATACG |
| | CC (SEQ ID NO: 54) |
| MA0063 | CCAGCCAACCTCGTCTTCTCYCCGCCATGGCTGCTCACC |
| | TA (SEQ ID NO: 55) |
| MA0062 | AACTGGTGTTATATCTTTTGKAACAAATTTGTGCTTTAT |
| | GT (SEQ ID NO: 56) |
| MA0045 | CATCCCAAAGCACTGAACGGMTATGCCATTACTTTGACA |
| | AT (SEQ ID NO: 57) |
| MA0070 | AATTTTCTAAAAAGATTGGCAGCAATTAATKGGGGCTTA |
| | TAGCGGTAACTTTGGTTYGCAT (SEQ ID NO: 58) |
| MA0071 | RGTTGAATATCGATCCAACTCGMCGYGGCWYGGTCGAGC |
| | TCAAGCTAGCTCCGCTCAGCTC (SEQ ID NO: 59) |

1. A method for identifying a maize plant or plant part, comprising screening for the presence of a polynucleotide comprising molecular markers MA0045, MA0062, MA0063, MA0070, MA0071, and MA0064, and optionally PZE-108095339 and/or Affx-91328160 in a maize plant or plant part.

2. The method according to claim 1, wherein said screening comprises screening for the presence of one or more of molecular markers MA0045, MA0062, MA0063, MA0064, MA0070, MA0071, PZE-108095339, and Affx-91328160.

3. The method according to claim 1, wherein said polynucleic acid is flanked by and includes molecular markers MA0045 and Affx-91328160, MA0045 and PZE-108095339, MA0045 and MA0064, MA0045 and MA0063, MA0045 and MA0070, or MA0045 and MA0071.

4. The method according to claim 1, wherein said method comprises screening for the presence of one or more molecular marker alleles selected from:
MA0045, which is a SNP which is A at a position corresponding to position 156,789,751 bp of chromosome 8 of the B73 reference genome AGPv4;
MA0062, which is a SNP which is G at a position corresponding to position 156,772,431 bp of chromosome 8 of the B73 reference genome AGPv4;
MA0063, which is a SNP which is T at a position corresponding to position 156,543,061 bp of chromosome 8 of the B73 reference genome AGPv4;
MA0070, which is a SNP which is T at a position corresponding to position 156,694,565 bp of chromosome 8 of the B73 reference genome AGPv4;
MA0071, which is a SNP which is C at a position corresponding to position 156,694,641 bp of chromosome 8 of the B73 reference genome AGPv4;
MA0064, which is a SNP which is C at a position corresponding to position 156,541,890 bp of chromosome 8 of the B73 reference genome AGPv4;
PZE-108095339, which is a SNP which is G at a position corresponding to position 156,378,424 bp of chromosome 8 of the B73 reference genome AGPv4; or
Affx-91328160, which is a SNP which is G at a position corresponding to position 156,372,823 bp of chromosome 8 of the B73 reference genome AGPv4.

5. The method according to claim 1, wherein said method comprises screening for the presence of one or more molecular marker alleles selected from:
PZE-108092843, which is a SNP which is C at a position corresponding to position 154,456,106 bp of chromosome 8 of the B73 reference genome AGPv4;
PZE-108094590, which is a SNP which is G at a position corresponding to position 155,860,812 bp of chromosome 8 of the B73 reference genome AGPv4;
MA0043, which is a SNP which is A at a position corresponding to position 155,999,733 bp of chromosome 8 of the B73 reference genome AGPv4;
MA0025, which is a SNP which is G at a position corresponding to position 156,967,599 bp of chromosome 8 of the B73 reference genome AGPv4;
PZE-108096469, which is a SNP which is G at a position corresponding to position 157,266,475 bp of chromosome 8 of the B73 reference genome AGPv4;
PZE-108096610, which is a SNP which is G at a position corresponding to position 157,375,673 bp of chromosome 8 of the B73 reference genome AGPv4; or
PZA-003182005, which is a SNP which is A at a position corresponding to position 158,167,578 bp of chromosome 8 of the B73 reference genome AGPv4.

6. The method according to claim 1, which is a method for identifying a maize plant or plant part having increased resistance to NCLB and/or a method for identifying a maize plant or plant part having increased resistance to Helminthosporium turcicum.

7. The method according to claim 1, which is a method for identifying a maize plant or plant part comprising a fragment of a HT2 allele and/or a HT3 allele.

8. An isolated polynucleic acid comprising a molecular marker selected from MA0045, MA0062, MA0063, MA0070, MA0071, MA0064, PZE-108095339, and/or Affx-91328160, the complement, or the reverse complement thereof.

9. An isolated polynucleic acid capable of specifically hybridizing with a polynucleic acid comprising a molecular marker selected from MA0045, MA0062, MA0063, MA0070, MA0071, MA0064, PZE-108095339, or Affx-91328160, the complement, or reverse complement thereof.

10. The isolated polynucleotide according to claim 8, wherein said polynucleotide has a length ranging from 15 to 500 nucleotides, preferably 15 to 100 nucleotides, more preferably 15 to 35 nucleotides.

11. The isolated polynucleotide according to claim 8, wherein said polynucleotide is a allele-specific primer or a probe.

12. The isolated polynucleotide according to claim 8, comprising, consisting essentially of, or consisting of a polynucleic acid having a sequence of any one of SEQ ID NO: 4-51, or a fragment thereof comprising at least the 15 most 3′ contiguous nucleotides thereof, the complement or reverse complement thereof, or a polynucleotide according to claim 8, comprising, consisting essentially of, or consisting of a sequence of any of SEQ ID NOs: 52 to 59 or fragments thereof, preferably of at least 15, more preferably at least 18 contiguous nucleotides, the complement or reverse complement thereof or of the fragment, preferably comprising at least
R at position 21 of SEQ ID NO: 52, preferably as the most 3′ or 5′, 2nd most 3′ or 5′, or 3rd most 3′ or 5′ nucleotide;
R at position 21 of SEQ ID NO: 53, preferably as the most 3′ or 5′, 2nd most 3′ or 5′, or 3rd most 3′ or 5′ nucleotide;
Y at position 21 of SEQ ID NO: 54, preferably as the most 3′ or 5′, 2nd most 3′ or 5′, or 3rd most 3′ or 5′ nucleotide;
Y at position 21 of SEQ ID NO: 55, preferably as the most 3′ or 5′, 2nd most 3′ or 5′, or 3rd most 3′ or 5′ nucleotide;
K at position 21 of SEQ ID NO: 56, preferably as the most 3′ or 5′, 2nd most 3′ or 5′, or 3rd most 3′ or 5′ nucleotide;
M at position 21 of SEQ ID NO: 57, preferably as the most 3′ or 5′, 2nd most 3′ or 5′, or 3rd most 3′ or 5′ nucleotide;
K at position 31 of SEQ ID NO: 58, preferably as the most 3′ or 5′, 2nd most 3′ or 5′, or 3rd most 3′ or 5′ nucleotide; or
Y at position 31 of SEQ ID NO: 59, preferably as the most 3′ or 5′, 2nd most 3′ or 5′, or 3rd most 3′ or 5′ nucleotide.

13. A method of using molecular markers MA0045, MA0062, MA0063, MA0070, MA0071, MA0064, PZE-108095339, and/or Affx-91328160 or a polynucleic acid according to any claim 8 for identifying a maize plant or plant part.

14. A maize plant or plant part comprising any one or more of molecular marker alleles MA0045, MA0062, MA0070, MA0071, optionally MA0063 and MA0064, wherein
MA0045 is a SNP which is A at a position corresponding to position 156,789,751 bp of chromosome 8 of the B73 reference genome AGPv4;
MA0062 is a SNP which is G at a position corresponding to position 156,772,430431 bp of chromosome 8 of the B73 reference genome AGPv4;
MA0063 is a SNP which is T at a position corresponding to position 156,543,061 bp of chromosome 8 of the B73 reference genome AGPv4;
MA0070, which is a SNP which is T at a position corresponding to position 156,694,565 bp of chromosome 8 of the B73 reference genome AGPv4;
MA0071, which is a SNP which is C at a position corresponding to position 156,694,641 bp of chromosome 8 of the B73 reference genome AGPv4;
MA0064 is a SNP which is C at a position corresponding to position 156,541,890 bp of chromosome 8 of the B73 reference genome AGPv4; optionally further comprising molecular marker allele PZE-108095339, wherein PZE-108095339 is a SNP which is G at a position corresponding to position 156,378,424 bp of chromosome 8 of the B73 reference genome AGPv4 and/or molecular marker allele Affx-91328160, wherein Affx-91328160 is a SNP which is G at a position corresponding to position 156,372,823 bp of chromosome 8 of the B73 reference genome AGPv4, optionally wherein said plant or plant part does not comprise HT2 or HT3 molecular marker allele;
PZE-108092843, which is a SNP which is C at a position corresponding to position 154,456,106 bp of chromosome 8 of the B73 reference genome AGPv4;
PZE-108094590, which is a SNP which is G at a position corresponding to position 155,860,812 bp of chromosome 8 of the B73 reference genome AGPv4;
MA0043, which is a SNP which is A at a position corresponding to position 155,999,733 bp of chromosome 8 of the B73 reference genome AGPv4;
MA0025, which is a SNP which is G at a position corresponding to position 156,967,599 bp of chromosome 8 of the B73 reference genome AGPv4;
PZE-108096469, which is a SNP which is G at a position corresponding to position 157,266,475 bp of chromosome 8 of the B73 reference genome AGPv4;
PZE-108096610, which is a SNP which is G at a position corresponding to position 157,375,673 bp of chromosome 8 of the B73 reference genome AGPv4; and/or
PZA-003182005, which is a SNP which is A at a position corresponding to position 158,167,578 bp of chromosome 8 of the B73 reference genome AGPv4; and
optionally
MA0021, which is a SNP which is G at a position corresponding to position 156,535,845 bp of chromosome 8 of the B73 reference genome AGPv4; and/or
MA0035, which is a SNP which is A at a position corresponding to position 156,542,253 bp of chromosome 8 of the B73 reference genome AGPv4.

15. A method for generating a maize plant or plant part, comprising introducing in the genome of a maize plant or plant part a polynucleic acid comprising a HT2 or HT3 allele which is flanked by molecular markers PZE-108092843 and PZA-003182005, or a fragment thereof comprising the RLK1 gene and one or more of molecular markers MA0045, MA0062, MA0063, MA0070, MA0071, MA0064, PZE-108095339, and Affx-91328160.


SEQUENCE LISTING (59 sequences)

SEQ ID NO: 1 - DNA, 24632 bp, Zea mays
atggctgctcacctaccacgcctccccgtcctcctcctcgtcctcctcgctgcccatgtcgtctccacctccgcccatgccgagcctcctcttccgagcccttacagcacctccgcccatggcgagcctcctcttccgagcacttacaacgtctccatgtgctcggaatcgttctggtgcggcggcgtcgaaatccgctacccgttctatcttgccaacgcaaccgccgactacagcgggagctactactcctgcggctacaccgacttgagcgtttcctgcaaactcgaggtcgaggggccgacgacgacatggacccctaccatccgtctcggcggcgacaactacaccgtcaagaacatcttgtacgactatcataccatctcactggcggacagcgatgtgctcggaggcggcgagtgccccgtcgtccaccacaacgtcagcttcgacgagacgtggctgcacaaccccagcgccttcgacaacctcaccttcttcttcggatgccactgggggccacgcgatacactgcctgaatttgccggcaacaacatcagctgcgccgggttcagtactccagctatcagcggtggaggctccttcgtgttcaagcctgaagatcttgacgaacatgcggagcaggagttggcttcacactgcgacgaggttttctccgtgccagtgagaagcgaggctctgcagcaggcgatcgtcagcaacctcagcctcggggacgggtacggcgagctgcttaggcaggggatcgagttggaatggaaacggacatcggaggatcagtgtggccagtgcgaggaatcgggctccggcggacggtgcgcctacagccagaagagagaattccttggctgcttctgcagcggagggaaggcgggcaacccgttctgcaaaccatcaagtaaagtcctgaaccgaccctcccttatttttttttcattttttgcaatcaccagagcacgcatcggttgcgtcagtatcttgcaacctcgtagctagccccgcagtgtcccctgtgtgcgagtaccgcgctgctccagcttgcctcctgctaacgcctaacggtgaatgcttcatgcttgacatgatctagctagtctacactttgcttggggtttgcctgggagctggaaattctggctcctgtttgcatcactcgacaaggacgctttcagacttgcgactctcgttctgcttttgcaccaaatccgtgttttttcatttcgtgatcgagattaatctagcgtagagatgtcaatgggtatccgaaactcgaaacccgatggatttttactctattaggtacaagtttgaatcaattttcatatcatagatttgttaataggcacaaatctatatccaacaggttcatagatacgggtttgttcctacagtaatcaaatccgtaaacccatgagttttttagacccgaccaaacctagcgtatattgtcattttattttataaacgaacaacaaaattattatctctctatttacttcctatttttttatcggttggtgaatgtataagtagttggtgagagtgttgcttgcttgctattataatatttagttatttactagtgttatatatgtggtggatggataacttattgaaaaggtcacttgattatacaacttattatttgtattcattctttctactaataatttttatatcaaatcatgaactcggtgtttatcatataaattttggaccatgatcttattaatcatcacgatagttattaattatgagaaaaacaaatatattggagataaaaccctcggctaacccgttaacccgatgggcacgggtttgaacaaaatttcaaatttattatgaatacaagttttttaacaaatatagatatgtttcatggatagagtttgagatgacaaaatccaacggatttgtatccgttgccatctctatccggcggcccttaccgtgctccacgagcagaggtcgtatcgtccctcttcccgtgtcgcctgcttcgcgttgccgaacggagacgtttggtagcgttggccggctctagcagtcgggtcaatttttttgttgttgttttcgatgttgttggatttttgttccgtataagccatgttttggtaatttatttagtccagccgaatccgaagacgtgtttgctgggttggagactttggagttgctagtcatgatatgctttctactcggtttgatttcaacccagttaggctatatttgatactctagtatttattccaatataaatggtttgaaagggattaagatataaattagtttaatttatatctttaatttctcttaacccatatgtattgggctgaatactaagtatcaaaacaagcccttatttaagatgcattttctttacagttacacgtggccactattgtgtggggtaggccgatcctacttgtcagtgctcattcgagagcaaagataccgaaggagattagagagactaaaagctttttactatttaaaattagataataagacgatttaatcccgttccatatctttgctctaaacaaaccctcaatgatcatatatctcggaagatccggccggctgttctttatttatcaagtgatgactgctgaccgcttatagaatatatattttaaagcaaaatttcttctacatcagtaaaaggactagacgaaacaatgatgcatttctctaacaaaagaaagtagaattatcaagcggagagccaagaccaaaagccttacatctatgggcgtcaaccaatgataccgcgaaccaccccagctggtctatactgtctgtcacgacccagcgagtaaccgtgtggctacgctgttagcttaggattgaatgaaatgctcggttcattaataaatcagctcacgagttaaatgatttattatataacgaaactatatgcatattatttaccagggtgacgacagggggcataggtgggcaatatccaccctaatgctcctctaattctatagggattgttaatttatttagctaattatcatgtaaacgatataaaaaatgtttctaacagccccacggtccaccctaatcttagaccctagcttcgcctgttacgataaatatgttatacatgtgtagtgtctatgtcctatgaattaaactaatgattgatgaactgtgcttatatattaaattggtctatgcgaatataactatgtgttaaactgatgaacatgtgtgtgaattgtgaattgataagtgatgagttgtgtctaatttagtgttatattgatgtgttttgtgaaactatgagtataattaatattttctataattaaatttgtttaaaattaactagaaattgattattatatatatatattgtttttctgctctaattcgcaagctaaacaagcaagctcaagctcgtaaacgagccgaaccgagctgactctgtggctcattaacttaacaacccgagccggaccaacttgttagcttaacgagccagctcgaatacggacgagttgagccgagctggcatgatatccagccctaattaggctcgtaccagtgcaacatatccctctcgcctttgtcacgtccagacatgtcaatgggccccgattcccgcaaggaattcctctattaagggatgatgatgagaaagtttctcccccacggaaaaattctctcctgacgcataaacggggacaacactccacatccccattcctcgtggggacctattaaacttacatatggtgatgttttcatgtaaaagttaatgataaaaataaataattacgttgtcaagatatcactttttgtacaaatgtgctcattctgatgtagacatattttttcttacatctaacaatctgtatgagtgagaatgttttgtattaataacaaagtaagtatgttctaattataatttaagcgggtccggggatccccgatggggatttatacccttgggaaacgaagatggggaagaaatgtcctacacaagttttcatggcgatcctcgcggggaactttttcgtcgtggggatagggatggggagctaaaacccgatgaggaattcctcgttgtcatccccagtcacgtcaactctcgactctactattgtctctgctgcatagtctgtggtaaactatagcctcagcctatagcacatcagcttttctatttcagaattaagtaattcatgcagttttatttcaacgctcgaaaattacacaacataatattttttattgaattaaaaattataaagtgcatacaaatcaataaaaatatattataattaaaacatatataaatctaatcggagacctcgatcttatcctcgtcttccaccacctctttttgttttgcttctggttcctcttcttcgtcatcaaccagaccacgtcctttttcgaacatctttatggccttggcatgggtttatctttcagctaggtccatatccctggttgaatggctctttagagtattccattgctttaaaagcttggtcttcactaatccaccccacaagttcttcattttaaatgcttcacttgttgactgttgcttgtggcttccttccctttccccttcaccttcatctttcgcccagccgcctttgccctatcacgccctactgatcggggcacttccttcttggtatctttagtgcctccagaactatattcactagagacactgagtcgggttatcttattcgtagtcccattttatcctagaaccttgtcattgctgcctccatgaccattgcctcgtctgcttcgctctgatgcaacttttcttctcgaaggtagtatccattaaacttgctcacctcccggttgtaggtgccctaatgatttttaagttgcttggcggtttgaggatatcaagggtcggaggtggagttatatgttgccgctatttgactccaaaaacatgatccagacttattgttgacaggggttgaattattagaatgcatattccaagaattaacctataatataagtaaactacaaatataaatattaaataaatgagaaatgattcactcaccaacttttcctcatagtcattcatccattctagcctctttggacacttttctttcggcccaggattttcctcggcttggttttcaaatccaacatactagacatgtatgtggaattggaggaggtgctcatacagaggtggagcatatgcaggaggggcatacagaggtactgagaaagaagtttgacttccatccgcagcaggtggaggggcatacagacgtaccgaggaaaaagcttgaattctggcttgggaagttggaggttgaccatatgaaggcggcaagtatgaatacagatgataggaaaaggttatggtggtggtggacgcgcaactgaaaatagtgcatgggggactgaagctgaaacatcagagcgacaatgtagtggagagactcatcaaacggaggggtttgtgacccatcggactacaagagatccatgaaggtgttcaaatctggggaccaacccgatattgtgtgaacagaggggagtttggaagagagaaacgaaatgaaatggtgtgtggaatgagctcaacccaagtagaggtacttacaaagggattggggatgaaatttgtgattttttgcaattttttattttttggattccaaacgatcaaaaacaactagttgcaatagctagctgtgggggatagatatcccccgggtccactaagggagtaaaagacctcacgaaaggcccaagggcccaataaatcgtaaggtcattcttccgtgggcctggggagaaacaaccaacaaaacagagaagacgtaagaccggattggtgcaaacccggacggcccacaacgtcgaacgagtaaatcgcaacagagacccgactttcccgcgctggagcccccatgcaacggagccatgcgaggataagtcggcgagggttacgtagggataaactcaagaggttcactaccttttagctacttgttgttatcatatccacgtgtactgccccacggtcgagtatataaggcctagggggcaccccttcagaacgatcgaccctatcttacttagccacccacagcaactctctgtgtcttcaatccagagagccctcttgtaaccacgctcacatactcaccaggacgtagggtgttacgcatctctaagcggcccgaacctgtaaatcttgtccactgtccctcgtgtgatcggcacgaactgttttgctacagtcgtcgacaccgtcctactcctagaaacaccttgaggggcaaccccgggtgtgcggttggacccaaaacaccgacagctggcgcgccaggtagggggtgtgtcgacgatccaagctagctcaatggccatcaccttctacagcaagatcaccatgcgtcctggatccgtattctgcttcgggacaatctcatctgtagcggatgaagagggaattctacaccgtatcgcagatccgccggaaaagaagactcctccaacgaactccgaaaatgccggagaggcacaacctccagctcttcggaaaaagatcgtctccggaaaggcgggggccgagggctcgctgacccggaagactccgttgtctacttcctcgacgaaagaatggacacggatcacgaggaagaaggagaccggggaaaagcaagtcgttctttccgttcctccggtctcaaaggagaacggaaagaaagtcgccgcgacagcagcaccattctaccccgacgtcctcttcgtcgggagagtggagtcgcccaccgtctctgacgacgaaccgaccgcgcccggagaagagccgcctcaacgagaatcccgccgacggagaaaccggcgcaggaacgttcgacgacatcacgcggccggagaacgggatccggagcagcctgtctcgcgagacgaggtctcggagataggagaaactccggaagaacgcgtcttcagagaacgaaggaactcccgacgacgtgatcgccggcgggctcaggaacaggccgagcaagatgcaaggcaacgccgggagaatccgctcttcgggcgcaacctaaatcccgacttcgcccgagctatgaacacgccaagcgaagtcggaggggtactagcttggatagctgatggacttcctcgaactcccgacgccgagggataccgacgcctgttcacccaggcagccaaccatcttctacctctcgctcacccgccgaacgatctacgacacgccatcaacagtcgccgagacgcgcgaagctccatcaatgcttcgcgcgaacggcggcatgagaacgagattcgccgtcgggaggaatacgaccgggatcatggcatcccgactcagagccaggccaccaggactgagtcggcaacagcctcaactggcggaaccacccggggacggccgaggaaccacaatcacaactcccctccccgggacagacatcgtccccgacaacaggaggacacgtgcggagtgtccgctcttactccacgccttagggccatccaatggcctcccaacttcaaggtatccaatgtcgacaaatatgagcctaagcaggatccagggggttggctagccgtctacaccaccgctgctcgggctgtcggagcatctgaggacgttatgaccgcgtatctgcccatcgtccttgggcaagatgcgctacagtggctacgacatttaccccgacactgcatcgacgactggggagacttcagtcgacgtttcaccgccaacttccagtccctctccgacaagccagcgcaaccgtgggacctcaaatccatcaagcgtcggggggatgagactctccggtcgtacctcaaaaggttccagaccatgagaaatcgcatccccgaggtcacggaggcggccgtgattgaggacttctacagaggatccaacgactcggcttttgtccgagccatactacagaaagcgccgactacttccgaggagctgttccgggaagccgacctctacatcaccgccgacgagcgagctcaggacctcatcggaggagcgaagcctgcaccggcagcaccacgacgcgacgcgaaccaaccacccgacaaacgctgggagaagaggcctcgcgaagaagtacacgccgccggaccacctgcctctcgcgcccgaggaggacctcgtggaggcgagcgcacgctggacgacatcctcgacgcccagtgcccataccacaaggacatgcgccacactcttcgcaactgccgggacttcaagcactccgttgggcatggccgacccttccaacctctacctcctcctccgccgaggggaggaccggatgaaccgcgacaacctcatcagccggaggagggaggaggaggagctttcccacgcgtcgacagggaggtcaacgtcatcttcggcggacatgggtcgcaggagaacaaaagacaacaaaagctcaacgaccgccagatactggtggcgaccaccggtcctcccgccccataccggtggtcagaacacccgatcaccttcactcgggaagatcaatggctcaacttcgaccatccaggcaaatacccgctcctcgtcgatccggtgatccgagagagccgggtgaagaaggtgctagtggacggggggagcagcatcaacatcaccttcccccggacgctccaaggcttgggggctcacctcaaagagctccacaagtcggacactcctttctttggcatcgtgccgacggagggagaatacccgctggggcacatctacatgccggtcaccttcggaactccggagaactacagaatcgagttcctgaggtttgaagtggcgaacttcgactgcggatacaacgccatcattggaagaccgggattggccaaattcatggccattccgcactatacgtacatgatactgaagatgccaggaccgcaagggatcataactgtgcgcgccgacttccaaggcgccgcagaatgtttccgagtggccatccaggcagccctcaccaccaagccgccaacggtcccctctacactggcgaattctaagcctgaggaggacctcgccgtaccggcaaacgaagctcaggccgcgacctctatgcggccgactgaagaaaccaagcgaatcaacctggggttcgctgatgagcgcaagacggccatcatcagctccagcttgaacgacaaataggaaggcgcgctcgtccagttcctgcaagataaccgagatgtattcgcatggcaacctgcggatatgccgggagtcccaagagaactggccgagcacaagttgaaggtttatccccaggcgaggccgatccggcaaaagctacgtcgtttcacgcccgacaagagagaggccatttgcgccgagttagctcgcttggttgcggctggatttattagagaagtattacaccccgagtggttagctaaccctgttcttgtactcaaaaagaataaagtggattggcgcatgtgcgtcgactatactgatctcaacaaacactgtcggaaggatcccttcgggctcccgaggatagatcaggtggtggactctaccgccggatgttctgtgctgtcattctgagattgctattccggatatcatcagattagtttggcaaaagaagacgaggagaaaacggcgttcatcactccgtttggtgctttctgttacacctccatgtcgttcggcctcaaaaacgctggagcgacttatcagagagccattcaaacatgcttagccaatcactggggcaagcgtgtggaggcttacgtagatgatgtggtgatcaaaacagagaattcggaaaacttcatcgaagatttacagctggttttcaacagcctgagaagatatagatggaagctcaatcctgaaaaatgtgttttcggggtaccagcaggaaagttgctcgggtttatcgtcagccactggggaattgaggctaatccagataagattgaagctatcatgaagatggaagctcctcggtcacaaaagaaggttcagcgacttactggatgtatggcagctctgagcagatttatatccaggctgggagaaaaaggtttaccattttacaagctactcaagaaggtggataagtttcaatggacttcagaagcacaagaagctctagacgcactgaagaaattcttgacaacagcaccagtactgaaaccaccccggcgagctacgccgactcaaccggctgaagatttgctgctgtatatctcttgcacgactcatgtggtaagcaccgcgttggtagtcgagcgagtagaagaaggacatgcctatccagtacaacatcctgtttatttcatcagtgaagttctaggcccctcaaagaaaaagtatcctcaggttcagaagctattatatgcagtacttctaactgcccgcaagctgcgtcactactttgacgaccacaaagtcatagtagtcactggttttccaataggggacattcttcacaacaaggaagccattggccgaatagccaagtgggcttgcgagctgggatcccacgacatcgagttccgacctcgcactgccatcaaaactcaagcactggttgatttcgtatcagagtggacagaacagcaagtaccagataatccagaaactgcagaagtatggcgaatgtattttgacggctcgctgaagctgcagggagcaggggcagggattctcttcactgcacctggaggcgaacacctcaagtacgccctctagttgctattcccggcctctaacaatgcagccgagtatgaagctttgatacacggattgaacatcgccatatcactgggcgtcaagagattgatggtatacggagattctctggtagtcatcagccagataaacaaggaatgagattgttcaagtgattcaatgggaaaatactgcgctgccgtccgaaagctggaagataaattcgagggtctggaatttcatcatatagaaagagatcgaaacacagcagccgacgtactgtccaagctcggatccggtcgaactcaggtcccaaccggagtcttcgtgcaagaaattctgcagccgagtatctcaatggatcaaacagaagagtgcaacgtcataaatcaacctgagtcagactctgatgactggagaaggctgattattaagtacataaagaatgacgaagaaccagacgacaagaactcggcagagcgcattgccaggcagtcggctcgctatacactcattggggaggagttgtacagaaggggcgcatcaggcatcctcatgaagtgcgttctcccgtccactgggaaacaacttctggaggaggtccacgcagggcaatgtgggatacatgcagcatccagaacactagtcgggaaggttttcaggtcaggattctattggccgacggcgaagaacgatgcagccgagttagttcagagatgcgaagcttgccaatacttgtcaaagcaacagcacctgccagcacagcaactacataccataccagtaacctggcctttcgcatgctggggactggatatgattggacctttcaagaaagctcaagggggatatactcatgtattggtggcgattgacaaattcactaaatggatagaattcaagcccattgcttctttaacctctgctaaagccgtggaattcatacaagacataatattcagattcgggatacccaacagtatcatcactgacttaggatccaactttaccagttcagaattcttcgacttctgcgagcaaaagagcattcagatcaagtatgcatctgtagcacatccaagagccaacgggcaggttgagcgagccaacggaatgatattggaggcactcaggaaaagggtcttcgacaagaatgaaaaattcgcaggaaagtggataagagaattaccttatgttgtttggagcctaagaacccaacctagccgagccctgcatggaaacactcctttcttcatggtctatgggtcggaggcagtgctacctgctgatctcaagtttggggcgccaaggttgatcttcgaaagcatagcggaagctgaggccaccaggctggaggacgttgatgtacttgaggaagaacggctgaacgcggtaatccaatcagcacggtaccagcagactctaaggcgctatcacgacaaggctatgcggcaacggttcttttcagtaggagacctcgtcctccgccgaattctaacgggagagggacggcacaagttatcacccttatgggaaggacccttcataatagcagaagtcactcggcccggatcgtatcgcctcactcagatggatggcacagaagttgggaactcctggaacatagagcacctcagaaaattttatccctagctgtatttcaaaactgctggggcgacaatgtattctgtaaagtggaaatatgtcgtcaataaagagagatttcaaagatactcggctcgtttacaattgacttgcattctcacttaactcggggtgaccactatgccctacaaacggagcaatcggcttaagtcggcaacgacttaaggcgatgcaacatgctcacgcttacttaacccgggatgaccactatgccctacaaacggagctatcggcttaagtcggcaacgacttaaggcggtgcaacatgctcacgcttacttaacttagggtgaccactatgccctacaaacggagcaattggcttaagtcggcaacgacttaaggcggtgcaacatgctcacgcttacttaacttagggtgaccactatgccctacaaacagagcaatcggcttaagtcggcaacgacttaaggcggtgcaacatgctcacgcttacttaactcggggtgaccactatgccctacaaacggagcaatcggcttaagtcggcaacgacttaaggcggtgcaacatgctcacgcttacttaacccgggatgaccactatgccctactaacggagctatcggctaaagtcagtggtggcttaagccggcgcaacatactcgcgcccatataattcaggggacgacttccatatcccgccaacaaatttcatgatcatcatgcgtcattaccacaaatttacgaaccaataaattctgatacgataagagagtaattatgtcattcgaatgataaactgatgtattttaacaaatactcgatcatgctaaccgcgcgctcagagcacgcaaaatgtttctctatttcgcaatgtctttttcaggaacgaccgacgaagtaggacgattcgaggaatcgggggcagcattcacgtcagcctttatgtcttcaggaacaaccacgccgggtctcctcattaagattcgtccagcacgaatagccttgtccatggctttatcgcgctgggctttgagagtagtcgcagtttcttcggcaactttagcagccagccgagcagcctctaactcctgggcaatggatttcttggaagctcggagttctttgatggtggcatccttcgctgatatcacttgcctaaggcgggtcacttcgtccgcagattcttgcagcctacaacgctgattgtccagttcctccattgtaaagcggtgactcctctccaagatggtcagcgagttgttagaatcacgatacaatctgtctagattgtcacgagaagcagcaaggatacgtttttcttcctacaaacaaaaatcaactccttcagaaatcaagcaagtaataacagcacacggttttgtaagttaccttttcagagtcaagccgagcatgaagagaagaactcagggcattggcatcatccaaagcagcggagacctgatttaattcagtttgactctgagagtacttctcctcgaagtcagcgcaccgttgaaccatttcagcctgctctcgggagtgtttttcctcaagaactgcaatctgctgagtcattcctatcaagaggtaatcaaacaaaatgaggtcagaaggtaaaacagtccacgaacaaaggcgaagaagaataagaaaaaagggcactaaccagcgttagttgcctccaactcggatacacgacgatgaagcgacactggattccaacacgcaagagacgaagccacttctggggcggaagcaccctgcgatctcagctggctggccatcccatctaccaaagcctgataagaagaacagatgaacataatgacaaaagttcacgcaacataaagaacaagctaaaatacagcacagtacctggaggttggaaaagaacgaagggatccccaaatcaggagaaatcagctgataaccaggcgcaaggccaccacccgaagcagcttgcaacgaaccagtaggaatcagttcagtgccttcaacagagccaaacgcccgatcagcggggacgctgccctctaaaatgcctctctggtccaaagtttgggctaccaccatgcagccagagtgtggaggtgaacccacgtgaacgtccatagaagtgcaggaaggagaccctgctcgggcaccctcgggggctgggtcatagttggcactgcccacttgagccggatcatccccggcagaaccctcgggggctgggcagtggcttacattcttcacccgagctaagtcatccccagcgacaccctcgggggctgggcatgtgtcagcaccattcttgagatccaggctctccgcagtaaccacctctaaggttgacgggccttcagccacttccatggaaccaggacaatccaagtcagcatcccgaccctcgagatcgcgctctaaggtcgacgaggcacgggatgacctcaacccagcatcaggcacggcagcgcaagcctccgttgcatcatcatccactggctctgataacaagtcctctgggaccatatcctcaagagtttgatcaaagttggccagggacagctcttgcagaccaatgagggcagacaaagcaggagaaggaactccactacttccaccgctggcgatgaactgccgactgtttttccgagttaaaggagcttcattttcttcctcctcatcttccacagtgacaacacaagcagcacccccattgggatcagcaacagatggagcacccacattgggatcagcagcagtttgggtacacccattggggtcagcgtcagtaaggccagttgcaggcacttcttcaacagcaggaactaaggtaccggcgtcctcatcaaaactggatactcgccggaggcgtcttctcttcttcttttgctcatcagcagaaggctcaggctgactggttcggctagggcgcctcgggcgagtattgactggtttctggacatccaaagttgtatcaacctctggaaggggcaccactgccaacgtaccagcaggagcagcgtcggaggaatcctcagctagcagatcaagcatggcatttatgtccgcatcactagggttcaggggcacctcaaaatggacctgccgttcgtcatcaggaatctccccaagcgaagcaaccaaggtaccgacttcttcagcagaggatcgcactctaaggcccaaattgccatcagtcacaggcggatttgacacaaaaagggtgaaagctttgggaggaggcaggttccaggccgagtatgccactggagcaccaacattcgaaaccttacccctgaggatcatttcaagtcggcctaccaagtccacaggaggaattctcctattggtaacccgagttgagtcggctaaccctcgatacagatatgctggataggccctgtctttcagtggctgaatgttcttaaaaacaaagtcagtgaccacagcttcagcagttagacccctctctttcagcagtccaacttcagttagcaacacacctgcctcagccacttcctgatctgtgggagactcagtccagctcggagtgcgaacgtctggctgtcttcccgaccggggggggggggaatttccataattatcaactatgaaccactctagacgccatcccttaatactgtccttgaggggaatctcaagatactcagttttccgcccgcggcgcatctccaaactggcacctccgaccaactgatgctgtcccccggccatcccagggcgacaatgatacaaatacttccataagccaaaatgcggcagcacgccaagaaaggcttcgcataagtggacgaaaatggagatttgcagaatggaattaggattcaaatgggtcaagttaatgtggtagaaatcaaggaggtcgcggaaaaagggagagatgggaaggccgaggccgcggagaagaaaaggagcgtaaactacagactcgtgggtatcctctgtcgggacagtagtcccgtggcaaatccgccaagaacagagttctcgcggaggaagaacctcgatggatacgaggtggagaagttcagcttcggaaatgatggacatatggttacctgcgaagggcaactggctgttggggtcgattggagggatcaccgcagcagacgagctcgcagttttcctcttgggcgccatcttgattttcaccagcgaacagaatagggggcgaatggcagagaggattcagaagcgggaatgcaagaactagggcacggaaagcaaaggcggctaaaagcgcagctcttatgggatattctcgagccagataccgtttcaaaagtgcccagtcatcacccgacggttatttccgaaaccccagcatgccacgtggtcatctggcagttatttccaaaaagccgacgtgtcacttcatcactcggttatcatcctcaaaataactcatgcggccggctatcactcggcgttgactctaaaacgctgatttcatattcagtcgctcggcattacctctaaaatgccgacgtcaccatccagacactcggtgttgactctaaaacgctgattttttattcagttgctcggcattaccactaaaaagccgacgccgacctccagtcactcggcgttgcctctaaaacgctgatctcgtattcaatcgctcggcattacttctaaaatgccgacgttaaccttcaatcactcggcgttgactctaaaacgctgattttatattcagttgctcggcattaccactaaaaagccgacaccgacctccagtcactcggcgttgcctctaaaacgctgatctcgtattcaatcgctcggcattacttctaaaatgccgacgttaaccttcaatcactcggcattgcctctaaaacgctgatctcgcattcgatcgctcggcattacttctaaaatgccgacgtcgaccttcaatcactcggcgttgcctctaaagcgatgatctcgtattcgattgctcggtattacttctaaaatgccgacgtcgaccttcaatcgctcggcgttgcttctaaaacgccgataccgactacaagattactcggcagctacttctggaagcgtcagcgtgatacccaagttattcatctactgtctcaaaagaatcagttttataatcaagcaaagcgagacatcaagaaggagccaacgtcattctttcattaagggaagacaataagtttacaaatcaactctttgcgagttgatgcttccctactattactacattacattactactactactatactatactatactactctacattactactacattattctaactacactatactaatatctaaagaccagcgacgcttagccactggccttgctgctgctgccaccgccgccgcccctggtgctgcccttgctgctgccgcctctggtgctgcccgtgctgctgccgcctctggtgctgcccttgctcccgtcaccgctgccgctgcccccgccgctgccgctgccgtgaccatcgccgtcgccaccgtcgtcgtcgtcgtcttcgtcgtcgtcgtcatcctcgccctcgccgtcgtccgagtcctcctcctccgaggagaactccgactcatccgagccctcgagcccggagcgctcgacggcccggatgtacgtccagacttccgcggcaatcccctgggagtcgtctgaatcatcagacgacgccgggggagagacgggagccagagacccctcggactcggaggagaactcctcctctgagtattccgagtcgccgaagtcctccgacggaggagtcggctcgcgcttgcgtttgttactcttgcccatggcggaagcagacaggagagaagagaaggatgcagtaaagaacagcgaagagatgtgaaaaaaccagaggagcaacggcgttatttatagaagggaaaggcaaccgctcacctccaaccgtggtcactaaacagtcgcaaagcattcaataagcacttccacccgtctgaagacgcgtcagacggctgacgccgtttcatgcaacactacacccattgggactccagtcaacagcgcaaaggatatgattacactcgccgtcccatcacattactactcagaagcagccggctaaaacactcagcgtaccaagccgtcccttgcccacacccattggggggacgcccaggaattatcagtatatttttcaaaacggtcacatccgtgcagggcacgcagtgaatacctcaagacaaaaatgagatatcagtaaggatcagaggaaagccaaatatagccagcaatgtacaagatatggccgacagaagcgacgtgaagactagacagagaacgtccgtcaaacgtccaatcagtcgcagaagcaaataaattgcactacctagcaagatatggatggattgcaaactcggctgctaaagacaaaatatacgctgacctctgcagcaaaatattgatgacataatgctgacctccaaagcataatgcaaatagcttcatgccaatttttacaaacgaaagaaagaccttcgaatggattatcctcaaaaatccatttgaaggtcgggggctacacccattgggtgcacctccggtgcaccccctggattatcattccaaaccgagatagtgccgacttctaaggcacgggacaagattaaaataccaatcctcgaccaagatgcaggagcacaaatggagataatttctggggaagaccttcgagtagattattttctaaaaatctactcaaaggtcgggggctacacccattgggtgcacctccggtgcacccaatgaagttcagaatcctgcaaatcgaaatgtcgacctctaaggcacaagcacgaaactaagcttcggtcaaatgagcgatcggagcaagagaagacagcaggaagtcattttttggacctgggatctttgggttgattacctctaaattaacccaaagatcgggggcttgtggtggatagatatcccccgggtccactaagggagtaaaagacctcacgaaaggcccaagggcccaataaatggtaaggtcattcttccgtgggcctggggagaaacaaccaacaaaacagagaagacgtaagaccggattggtgcaaacccggacgacccacaacgtcgaacgagtaaatcgcaacagagacccgactttcccgcgctggagcccccatgcaacggagccatgcaaggataagtcggcgagggttacgtagggataaactcaagaggttcactaccttttagctacttgttgttatcatatccacgtgtactgccccacagtcgagtatataaggcctagggggcaccccttcagaacgatcgaccctatcttacttagccccccacagcaactctctgtgtcttcaatccagagagccctcttgtaaccacgctcacatactcaccgggacgtagggtgttacgcatctctaagcggcccgaacctgtaaatcttgtccactgtccctcgtgtgatcggcacgaaccgttttgctacagtcgtcgacactgtcctactcctagaaacaccttgaggggcaaccccgggtgtgcggtcggacccaaaacaccgacactagcacacgtggaccaattgaaagcccccaccttgctctcgttcgtcgctcgttcacgccagcgcgtcacccacccactgcgccctctcatcctccttgcccgagaacgtgcttcagagcaggcgagagtggggcgatatcgcccgagggatgcatctctccctcctctcaccccgggccaggctacaacagccccctactgcccaaccactcgccctcgttggcaccaaccatactaccacaacactccaatgcacaatttttaatgttagcctatattcaccttagcatcacatcatatttcttctacaaaattatatttagaacgaaacacgacgttttgcttggaattttttcgatggagtagaaaaaatataatccaaacaaattacgtaaaatacgtaaaataaaaaatatagctcctaaaagttaaaattactcatcattatcaccctcctcgttttcttcctcgtctagttttaattgcgtgatcaatttttttagaaccaacctagaccctcctcataccagtggtggattgtttcagtaacttggtgaactggtcgtacaattttttgattggtttctcgtgtcactaaccttgaataattgcttcgttatggttttcttctggacaagggactcattggtactcattcctggtgcctaacccttgttttgtgctcttgcccaatcacgaccaatagggtatggtggtttggcactatcggacgatgtagagttataatcaccctccacgttgaggcaagatcattttctctcattgctagagtttgaatcaccatgtcacgtgcaccacttgaattgatgatgaagagcacgccattagtttcatcgagcctgttgtggaagagtagcataacattatttgatctgctcggtaggttatacgtggccgatatttggcaccaaaaggtcaaacatttttgttgtttccctcaaatcttttttggtctcaaaaaggtcaaacctttttttggctttccatccctgtccccacgaacagtcacgagtaactttttctcccatccccgttcgtgtgggggaatttatccccatgggaaatctatccctgttagagaatgtaatatttttggggcaaggtcaaattgataaaccaatataaattgaacaaataagatttaaaattacaagagaaatctactttagagttgagcttgctattttataaaaggctatgtgttgaatttttattataactttaacatatcaatggattgattttagattaagagaagttcgtgggggcggatatgcgaggaacagggatggggaatggttcaccattctcgtcccacgaacacgacaaggactaaatttttctcgtttcaatctccgtggggactaaattacacctatccccgtccactaataaagggataccccgcggggaattagggatcacgtccccatttcacatttttagacttctcacattgagcaacaacaaaacattttatgaattagtagccattttacttatctaaaacaattactattgaatcttaatcattttgtgcaaaataaagaatgaactaaattttgggtcgggtgtgggtcacccgtaggttgaaataaaaactcgcacccacactcgtgaaactttgggtcagatgagggttacacccgcggattaaaatctctacccatacccacacccgtcagatcgagtacccaaagattttgggtttgcgggttaaattgtcatccctagacacgagtgtgctggaggatttggaagtttcgcaatgcgtacttaccaagtgtttggttctatggattaaagtttagcatgtgtcgcattagatgattgaatgcctattatgagtattaaatattgtctgattattaatcaaattacacaagtgaaggctaaacaactagacaaatttattaagcctaattaatctatgattagcaaatgtttatagtagcagcatcttagtgaatcatgaactaattaggtttaataggttcgtcttaacgtttagttcttatctatgtaattaattttataagtaaactatatttaatattcctaattagtctccggatataacatagactaaaatttagtcagtggtgctgctgctggctccttttcatttcatccgacataatgttttcctcccaatttattgtgttgttttgtttgtgctttaaaaatgacttgtcctctgcttgctgtatcaaggtcgtgatgtgtgcttaacttattcccttccttcctagctgcccttagcatttatataggggtatttggttacatggactaaagtttagtttagtccatgtccaattataaaactaattacataactgaatactaaaataccaaaatacgagaaaaattattaaacctaattaatttattattagcaatgtttattgtaacatcacataagcaaagcatagactgatttagtttaataggttcgtcttatcatttagtcttgatctgtataattagggtatgtttggttcactgcctaacttaccacactttgcctaacttttctgcctaaggttagttcttcaatttgaacgactaaccttaggcaaagtatggcgtagttagccacgaaccaaacagtcccttagttttgtaattaaactatatttactatttttaactagtatccaaacattaaatgtgacatggattaaaatttagttagtgactttaggcttttagagttgagttagttgtagcttggtttagttatttgtttgttctcttgcgtttatttaggacctctctataatgttttatcaccttaatacaaattaaaatacgcagctcttgtgtattgcaggcgtgtgtgttcctatatttttaggctcaattatgaaggaaattattctcagcgagcatataaccgttttggaggtaaaatgaactaaaagcatataggcctgcctgtttgcataagtacaccctccgtctaaaaaagaataaaaatctcatttcttgatgagtcaaaaaagttcaaatttaagaaaatatatgttacgacacgaatatttataatgtgtaataagtaccgctagattaattttaaaataaaattttcataataaacctatttgaagatacaagtattggtactatttctaataaatctaatcaaactggtgttatatcttttggaacaaatttgtgctttatgtttctggttgacgtgaatcagcttaatcttgacgaaatctaacattgtcttttgttcgttggcatacaggatcaacaaaaaggagagaagcagcatctattgttggtaagagcctatagtcaatacccatgttcatttcgtctaaaagagcagaagaaaagcatatgatgaattattgccatgtcatgtttaaaatacagaattctcaaaaacaaaaacaaaaaaaaaaacttggaatccactaaccactgatagcattgtagaaaatttcatcctccctttgggcaatacactgatgagtttacatgctgactagtggtgcatttgttctttgccaattgagtttttagaatgctttgcagctgaattcacttgtgatgtttttttttgtgtgtgatgcaggtgctgttgccgttgcattcctgtgtctagtcattctcacatgcttcttggcttgtagacattgttcgctgcccttcaaatcgaagaacaaaccagggacaaggattgagtccttcctacagaagaacgagagtagtatacatccgaaaagatacacctacgcggacgtgaaaagaatgacaaaatccttcgctgtgaagctaggccaaggtgggtttggtgctgtatacaaaggcagcctccacgatggccgacaggtagcagtcaagatgctgaaggacacccaaggtgacggcgaggaattcatgaacgaggtggctagcatcagcaggacttctcatgtcaacgtcgtgacacttctagggttttgcttgcaagggtcgaaaagagcactgatctacgagtacatgcccaatggttcgctcgaaaggtatgccttcaccggtgacatgaacagtgagaatttgctaacctgggaaagactatttgacatagcgattggcacggccagagggctcgaatacctacaccggggatgcaacactcggatcgtgcattttgacatcaagccacacaacatcctgttagaccaggatttctgccctaagatctctgactttggactggccaagctatgtctgaacaaagagagcgctatctccattgctggcgcaagagggacgatagggtatatcgccccggaggtctactcaaagcaatttggaacgatcagcagcaagtctgatgtctatagctatgggatgatggtccttgagatggttggagcaagggacaggaatacaagcgcagatagtgaccatagcagccaatatttccctcagtggctttatgaacatttggacgactattgtgttggtgcttccgagattaatggtgagaccacagagctcgtgaggaagatgatagttgtaggtctgtggtgcatacaagtgattccgactgatcgaccaacaatgacgagagtcgtcgagatgttggaaggaagcacaagtaatctagagttgccacccagagttctcttgagctga

SEQ ID NO: 2 - DNA, 2004 bp, Artificial Sequence
  cDNA derived from Ht2 gene (SEQ ID NO 1)
atggctgctcacctaccacgcctccccgtcctcctcctcgtcctcctcgctgcccatgtcgtctccacctccgcccatgccgagcctcctcttccgagcccttacagcacctccgcccatggcgagcctcctcttccgagcacttacaacgtctccatgtgctcggaatcgttctggtgcggcggcgtcgaaatccgctacccgttctatcttgccaacgcaaccgccgactacagcgggagctactactcctgcggctacaccgacttgagcgtttcctgcaaactcgaggtcgaggggccgacgacgacatggacccctaccatccgtctcggcggcgacaactacaccgtcaagaacatcttgtacgactatcataccatctcactggcggacagcgatgtgctcggaggcggcgagtgccccgtcgtccaccacaacgtcagcttcgacgagacgtggctgcacaaccccagcgccttcgacaacctcaccttcttcttcggatgccactgggggccacgcgatacactgcctgaatttgccggcaacaacatcagctgcgccgggttcagtactccagctatcagcggtggaggctccttcgtgttcaagcctgaagatcttgacgaacatgcggagcaggagttggcttcacactgcgacgaggttttctccgtgccagtgagaagcgaggctctgcagcaggcgatcgtcagcaacctcagcctcggggacgggtacggcgagctgcttaggcaggggatcgagttggaatggaaacggacatcggaggatcagtgtggccagtgcgaggaatcgggctccggcggacggtgcgcctacagccagaagagagaattccttggctgcttctgcagcggagggaaggcgggcaacccgttctgcaaaccatcaagatcaacaaaaaggagagaagcagcatctattgttggtgctgttgccgttgcattcctgtgtctagtcattctcacatgcttcttggcttgtagacattgttcgctgcccttcaaatcgaagaacaaaccagggacaaggattgagtccttcctacagaagaacgagagtagtatacatccgaaaagatacacctacgcggacgtgaaaagaatgacaaaatccttcgctgtgaagctaggccaaggtgggtttggtgctgtatacaaaggcagcctccacgatggccgacaggtagcagtcaagatgctgaaggacacccaaggtgacggcgaggaattcatgaacgaggtggctagcatcagcaggacttctcatgtcaacgtcgtgacacttctagggttttgcttgcaagggtcgaaaagagcactgatctacgagtacatgcccaatggttcgctcgaaaggtatgccttcaccggtgacatgaacagtgagaatttgctaacctgggaaagactatttgacatagcgattggcacggccagagggctcgaatacctacaccggggatgcaacactcggatcgtgcattttgacatcaagccacacaacatcctgttagaccaggatttctgccctaagatctctgactttggactggccaagctatgtctgaacaaagagagcgctatctccattgctggcgcaagagggacgatagggtatatcgccccggaggtctactcaaagcaatttggaacgatcagcagcaagtctgatgtctatagctatgggatgatggtccttgagatggttggagcaagggacaggaatacaagcgcagatagtgaccatagcagccaatatttccctcagtggctttatgaacatttggacgactattgtgttggtgcttccgagattaatggtgagaccacagagctcgtgaggaagatgatagttgtaggtctgtggtgcatacaagtgattccgactgatcgaccaacaatgacgagagtcgtcgagatgttggaaggaagcacaagtaatctagagttgccacccagagttctcttgagctga

SEQ ID NO: 3 - protein, 667 aa, Zea mays
MAAHLPRLPVLLLVLLAAHVVSTSAHAEPPLPSPYSTSAHGEPPLPSTYNVSMCSESFWCGGVEIRYPFYLANATADYSGSYYSCGYTDLSVSCKLEVEGPTTTWTPTIRLGGDNYTVKNILYDYHTISLADSDVLGGGECPVVHHNVSFDETWLHNPSAFDNLTFFFGCHWGPRDTLPEFAGNNISCAGFSTPAISGGGSFVFKPEDLDEHAEQELASHCDEVFSVPVRSEALQQAIVSNLSLGDGYGELLRQGIELEWKRTSEDQCGQCEESGSGGRCAYSQKREFLGCFCSGGKAGNPFCKPSRSTKRREAASIVGAVAVAFLCLVILTCFLACRHCSLPFKSKNKPGTRIESFLQKNESSIHPKRYTYADVKRMTKSFAVKLGQGGFGAVYKGSLHDGRQVAVKMLKDTQGDGEEFMNEVASISRTSHVNVVTLLGFCLQGSKRALIYEYMPNGSLERYAFTGDMNSENLLTWERLFDIAIGTARGLEYLHRGCNTRIVHFDIKPHNILLDQDFCPKISDFGLAKLCLNKESAISIAGARGTIGYIAPEVYSKQFGTISSKSDVYSYGMMVLEMVGARDRNTSADSDHSSQYFPQWLYEHLDDYCVGASEINGETTELVRKMIVVGLWCIQVIPTDRPTMTRVVEMLEGSTSNLELPPRVLLS

SEQ ID NO: 4 - DNA, 49 bp, Artificial Sequence
  Affx-91328160 HT2/HT3 allele primer
gaaggtcggagtcaacggattgggaagattcatatttgtttacatcacg

SEQ ID NO: 5 - DNA, 51 bp, Artificial Sequence
  Affx-91328160 alternative allele primer
gaaggtgaccaagttcatgctaagggaagattcatatttgtttacatcaca

SEQ ID NO: 6 - DNA, 29 bp, Artificial Sequence
  Affx-91328160 Common primer
ctccacagtttttactcccaagtttagaa

SEQ ID NO: 7 - DNA, 40 bp, Artificial Sequence
  PZE-108095339 HT2/HT3 allele primer
gaaggtcggagtcaacggattcttggcgtgctgcaggacc

SEQ ID NO: 8 - DNA, 38 bp, Artificial Sequence
  PZE-108095339 alternative allele primer
gaaggtgaccaagttcatgcttggcgtgctgcaggact

SEQ ID NO: 9 - DNA, 27 bp, Artificial Sequence
  PZE-108095339 Common primer
gtttcgggtttcagaagtcaaacggtt

SEQ ID NO: 10 - DNA, 47 bp, Artificial Sequence
  MA0064 HT2/HT3 allele primer
gaaggtcggagtcaacggattatgaacttctttttggtcgttttcgc

SEQ ID NO: 11 - DNA, 48 bp, Artificial Sequence
  MA0064 alternative allele primer
gaaggtgaccaagttcatgctgatgaacttctttttggtcgttttcgt

SEQ ID NO: 12 - DNA, 26 bp, Artificial Sequence
  MA0064 Common primer
gctagaacatggcgtatacggaacaa

SEQ ID NO: 13 - DNA, 40 bp, Artificial Sequence
  MA0063 HT2/HT3 allele primer
gaaggtcggagtcaacggattggtgagcagccatggcgga

SEQ ID NO: 14 - DNA, 39 bp, Artificial Sequence
  MA0063 alternative allele primer
gaaggtgaccaagttcatgctgtgagcagccatggcggg

SEQ ID NO: 15 - DNA, 23 bp, Artificial Sequence
  MA0063 Common primer
gtgaacccagccaacctcgtctt

SEQ ID NO: 16 - DNA, 51 bp, Artificial Sequence
  MA0062 HT2/HT3 allele primer
gaaggtgaccaagttcatgctatctaatcaaactggtgttatatcttttgg

SEQ ID NO: 17 - DNA, 53 bp, Artificial Sequence
  MA0062 alternative allele primer
gaaggtcggagtcaacggattaaatctaatcaaactggtgttatatcttttgt

SEQ ID NO: 18 - DNA, 29 bp, Artificial Sequence
  MA0062 Common primer
cacgtcaaccagaaacataaagcacaaat

SEQ ID NO: 19 - DNA, 44 bp, Artificial Sequence
  MA0045 HT2/HT3 allele primer
gaaggtgaccaagttcatgctaacatcccaaagcactgaacgga

SEQ ID NO: 20 - DNA, 43 bp, Artificial Sequence
  MA0045 alternative allele primer
gaaggtcggagtcaacggattacatcccaaagcactgaacggc

SEQ ID NO: 21 - DNA, 30 bp, Artificial Sequence
  MA0045 Common primer
ccaagaactctattagattgttgaatagca

SEQ ID NO: 22 - DNA, 28 bp, Artificial Sequence
  Affx-91328160 HT2/HT3 allele primer
gggaagattcatatttgtttacatcacg

SEQ ID NO: 23 - DNA, 30 bp, Artificial Sequence
  Affx-91328160 alternative allele primer
aagggaagattcatatttgtttacatcaca

SEQ ID NO: 24 - DNA, 29 bp, Artificial Sequence
  Affx-91328160 Common primer
ctccacagtttttactcccaagtttagaa

SEQ ID NO: 25 - DNA, 19 bp, Artificial Sequence
  PZE-108095339 HT2/HT3 allele primer
cttggcgtgctgcaggacc

SEQ ID NO: 26 - DNA, 17 bp, Artificial Sequence
  PZE-108095339 alternative allele primer
tggcgtgctgcaggact

SEQ ID NO: 27 - DNA, 27 bp, Artificial Sequence
  PZE-108095339 Common primer
gtttcgggtttcagaagtcaaacggtt

SEQ ID NO: 28 - DNA, 26 bp, Artificial Sequence
  MA0064 HT2/HT3 allele primer
atgaacttctttttggtcgttttcgc

SEQ ID NO: 29 - DNA, 27 bp, Artificial Sequence
  MA0064 alternative allele primer
gatgaacttctttttggtcgttttcgt

SEQ ID NO: 30 - DNA, 26 bp, Artificial Sequence
  MA0064 Common primer
gctagaacatggcgtatacggaacaa

SEQ ID NO: 31 - DNA, 19 bp, Artificial Sequence
  MA0063 HT2/HT3 allele primer
ggtgagcagccatggcgga

SEQ ID NO: 32 - DNA, 18 bp, Artificial Sequence
  MA0063 alternative allele primer
gtgagcagccatggcggg

SEQ ID NO: 33 - DNA, 23 bp, Artificial Sequence
  MA0063 Common primer
gtgaacccagccaacctcgtctt

SEQ ID NO: 34 - DNA, 30 bp, Artificial Sequence
  MA0062 HT2/HT3 allele primer
atctaatcaaactggtgttatatcttttgg

SEQ ID NO: 35 - DNA, 32 bp, Artificial Sequence
  MA0062 alternative allele primer
aaatctaatcaaactggtgttatatcttttgt

SEQ ID NO: 36 - DNA, 29 bp, Artificial Sequence
  MA0062 Common primer
cacgtcaaccagaaacataaagcacaaat

SEQ ID NO: 37 - DNA, 23 bp, Artificial Sequence
  MA0045 HT2/HT3 allele primer
aacatcccaaagcactgaacgga

SEQ ID NO: 38 - DNA, 22 bp, Artificial Sequence
  MA0045 alternative allele primer
acatcccaaagcactgaacggc

SEQ ID NO: 39 - DNA, 30 bp, Artificial Sequence; identical to SEQ ID NO: 21
  MA0045 Common primer

SEQ ID NO: 40 - DNA, 53 bp, Artificial Sequence
  MA0070
gaaggtcggagtcaacggattcaattttctaaaaagattggcagcaattaatt

SEQ ID NO: 41 - DNA, 53 bp, Artificial Sequence
  MA0070
gaaggtgaccaagttcatgctcaattttctaaaaagattggcagcaattaatg

SEQ ID NO: 42 - DNA, 25 bp, Artificial Sequence
  MA0070
  misc_feature: R=A/G
craaccaaagttaccgctataagcc

SEQ ID NO: 43 - DNA, 42 bp, Artificial Sequence
  MA0071
gaaggtgaccaagttcatgctgagctagcttgagctcgaccg

SEQ ID NO: 44 - DNA, 42 bp, Artificial Sequence
  MA0071
gaaggtcggagtcaacggattgagctagcttgagctcgacca

SEQ ID NO: 45 - DNA, 25 bp, Artificial Sequence
  MA0071
  misc_feature: Y=C/T
gcttatagcggtaactttggttygc

SEQ ID NO: 46 - DNA, 32 bp, Artificial Sequence
  MA0070
caattttctaaaaagattggcagcaattaatt

SEQ ID NO: 47 - DNA, 32 bp, Artificial Sequence
  MA0070
caattttctaaaaagattggcagcaattaatg

SEQ ID NO: 48 - DNA, 25 bp, Artificial Sequence
  MA0070
  misc_feature: R=A/G
craaccaaagttaccgctataagcc

SEQ ID NO: 49 - DNA, 21 bp, Artificial Sequence
  MA0071
gagctagcttgagctcgaccg

SEQ ID NO: 50 - DNA, 21 bp, Artificial Sequence
  MA0071
gagctagcttgagctcgacca

SEQ ID NO: 51 - DNA, 25 bp, Artificial Sequence
  MA0071
  misc_feature: Y=C/T
gcttatagcggtaactttggttygc

SEQ ID NO: 52 - DNA, 41 bp, Artificial Sequence
  Affx-91328160
  misc_feature (21)..(21): R=A/G
ttcatatttgtttacatcacrattctaaacttgggagtaaa

SEQ ID NO: 53 - DNA, 41 bp, Artificial Sequence
  PZE-108095339
  misc_feature (21)..(21): R=A/G
acgtggcgccagccacgcacrgtcctgcagcacgccaagca

SEQ ID NO: 54 - DNA, 41 bp, Artificial Sequence
  MA0064
  misc_feature (21)..(21): Y=C/T
ttcttttttggtcgttttcgytcctttgttccgtatacgcc

SEQ ID NO: 55 - DNA, 41 bp, Artificial Sequence
  MA0063
  misc_feature (21)..(21): Y=C/T
ccagccaacctcgtcttctcyccgccatggctgctcaccta

SEQ ID NO: 56 - DNA, 41 bp, Artificial Sequence
  MA0062
  misc_feature (21)..(21): K=G/T
aactggtgttatatcttttgkaacaaatttgtgctttatgt

SEQ ID NO: 57 - DNA, 41 bp, Artificial Sequence
  MA0045
  misc_feature (21)..(21): M=A/C
catcccaaagcactgaacggmtatgccattactttgacaat

SEQ ID NO: 58 - DNA, 61 bp, Artificial Sequence
  MA0070
  misc_feature (31)..(31): K=G/T
aattttctaaaaagattggcagcaattaatkggggcttatagcggtaactttggttygcat

SEQ ID NO: 59 - DNA, 61 bp, Artificial Sequence
  MA0071
  misc_feature (1)..(1): R=A/G
  misc_feature (23)..(23): M=A/C
  misc_feature (26)..(26): Y=C/T
  misc_feature (30)..(30): W=A/T
  misc_feature (31)..(31): Y=C/T
rgttgaatatcgatccaactcgmcgyggcwyggtcgagctcaagctagctccgctcagctc